SEQUENCE LISTING (283 sequences)

SEQ ID NO: 1 - protein, 15 aa, Artificial Sequence
  Description of Artificial Sequence: Synthetic peptide
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPADCMXDWSTPXWC

SEQ ID NO: 2 - protein, 15 aa, Artificial Sequence
  Description of Artificial Sequence: Synthetic peptide
  MOD_RES (3)..(3): 1-naphthylalanine
  MOD_RES (4)..(4): D-amino acid
  MOD_RES (7)..(7): Homoarginine
  MOD_RES (9)..(9): D-amino acid
  MOD_RES (13)..(13): Trans-4-hydroxy-L-proline
  MOD_RES (14)..(14): D-amino acid
CPADCMXDWSTPXWC

SEQ ID NO: 3 - protein, 26 aa, Artificial Sequence
  Description of Artificial Sequence: Synthetic peptide
  MOD_RES (3)..(3): 1-naphthylalanine
  MOD_RES (4)..(4): D-amino acid
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine
  MOD_RES (12)..(12): Sarcosine
  MOD_RES (13)..(13): Sarcosine
  MOD_RES (14)..(14): Sarcosine
  MOD_RES (15)..(15): Beta-alanine
  MOD_RES (18)..(18): Homoarginine
  MOD_RES (24)..(24): Trans-4-hydroxy-L-proline
CPAKXXXXXXXXXXACMXDWSTPXWC

SEQ ID NO: 4 - protein, 15 aa, Artificial Sequence
  Description of Artificial Sequence: Synthetic peptide
  MOD_RES (7)..(7): Homoarginine
  MOD_RES (13)..(13): Trans-4-hydroxy-L-proline
CPFGCMXDWSTPXWC

SEQ ID NO: 5 - protein, 15 aa, Artificial Sequence
  Description of Artificial Sequence: Synthetic peptide
  MOD_RES (14)..(14): Norleucine
CIEEGQYCFADPYXC

SEQ ID NO: 6 - protein, 15 aa, Artificial Sequence
  Description of Artificial Sequence: Synthetic peptide
  MOD_RES (2)..(2): t-butyl-alanine
  MOD_RES (5)..(5): D-amino acid
  MOD_RES (14)..(14): Norleucine
CAPEAPYCFADPYXC

SEQ ID NO: 7 - protein, 15 aa, Artificial Sequence
  Description of Artificial Sequence: Synthetic peptide
  MOD_RES (10)..(10): D-amino acid
  MOD_RES (14)..(14): Norleucine
CIEEGQYCFADPYXC

SEQ ID NO: 8 - protein, 15 aa, Artificial Sequence
  Description of Artificial Sequence: Synthetic peptide
  MOD_RES (2)..(2): t-butyl-alanine
  MOD_RES (5)..(5): D-amino acid
  MOD_RES (14)..(14): Norleucine
CAPKAPYCFADPYXC

SEQ ID NO: 9 - protein, 15 aa, Artificial Sequence
  Description of Artificial Sequence: Synthetic peptide
  MOD_RES (2)..(2): t-butyl-alanine
  MOD_RES (5)..(5): D-amino acid
  MOD_RES (14)..(14): Norleucine
CAPEKPYCFADPYXC

SEQ ID NO: 10 - protein, 15 aa, Artificial Sequence
  Description of Artificial Sequence: Synthetic peptide
  MOD_RES (2)..(2): t-butyl-alanine
  MOD_RES (4)..(4): 4-pentynoic acid modified residue
  MOD_RES (5)..(5): D-amino acid
  MOD_RES (14)..(14): Norleucine
CAPKAPYCFADPYXC

SEQ ID NO: 11 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine
CAPEKPYCFADPYXC

SEQ ID NO: 12 - protein, 15 aa, Artificial Sequence
  Description of Artificial Sequence: Synthetic peptide
  MOD_RES (5)..(5): 4-pentynoic acid modified D-amino acid
  MOD_RES (14)..(14): Norleucine
CIEEKQYCFADPYXC

SEQ ID NO: 13 - skipped

SEQ ID NO: 14 - protein, 15 aa, Artificial Sequence
  Description of Artificial Sequence: Synthetic peptide
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPAKCMXDWSTPXWC

SEQ ID NO: 15 - protein, 14 aa, Artificial Sequence
  Description of Artificial Sequence: Synthetic peptide
  MOD_RES (1)..(1): 3-mercaptopropionic acid modified residue
  MOD_RES (2)..(2): 1-naphthylalanine
  MOD_RES (3)..(3): D-amino acid
  MOD_RES (6)..(6): Homoarginine
  MOD_RES (12)..(12): Trans-4-hydroxy-L-proline
PAKCMXDWSTPXWC

SEQ ID NO: 16 - protein, 14 aa, Artificial Sequence
  Description of Artificial Sequence: Synthetic peptide
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
  MOD_RES (14)..(14): Cysteamine modified residue
CPAKCMXDWSTPXW

SEQ ID NO: 17 - protein, 13 aa, Artificial Sequence
  Description of Artificial Sequence: Synthetic peptide
  MOD_RES (1)..(1): 3-mercaptopropionic acid modified residue
  MOD_RES (2)..(2): 1-naphthylalanine
  MOD_RES (3)..(3): D-amino acid
  MOD_RES (6)..(6): Homoarginine
  MOD_RES (12)..(12): Trans-4-hydroxy-L-proline
  MOD_RES (13)..(13): Cysteamine modified residue
PAKCMXDWSTPXW

SEQ ID NO: 18 - protein, 15 aa, Artificial Sequence
  Description of Artificial Sequence: Synthetic peptide
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPAKCMXHWSTPXWC

SEQ ID NO: 19 - protein, 15 aa, Artificial Sequence
  Description of Artificial Sequence: Synthetic peptide
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPAKCMXEWSTPXWC

SEQ ID NO: 20 - protein, 15 aa, Artificial Sequence
  Description of Artificial Sequence: Synthetic peptide
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPAECMXDWSTPXWC

SEQ ID NO: 21 - protein, 15 aa, Artificial Sequence
  Description of Artificial Sequence: Synthetic peptide
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPAACMXDWSTPXWC

SEQ ID NO: 22 - protein, 15 aa, Artificial Sequence
  Description of Artificial Sequence: Synthetic peptide
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPAECLXDWSTPXWC

SEQ ID NO: 23 - protein, 15 aa, Artificial Sequence
  Description of Artificial Sequence: Synthetic peptide
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPAECMXEWSTPXWC

SEQ ID NO: 24 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
  MOD_RES (14)..(14): Homoarginine
CXLVNPLCLHPDWXC

SEQ ID NO: 25 - protein, 15 aa, Artificial Sequence
  Description of Artificial Sequence: Synthetic peptide
  MOD_RES (14)..(14): Homoarginine
CLWDPTPCANLHLXC

SEQ ID NO: 26 - protein, 15 aa, Artificial Sequence
  Description of Artificial Sequence: Synthetic peptide
  MOD_RES (2)..(2): Trans-4-hydroxy-L-proline
  MOD_RES (10)..(10): 4-pentynoic acid modified residue
  MOD_RES (12)..(12): D-amino acid
  MOD_RES (14)..(14): Homoarginine
CXLVNPLCLKPDWXC

SEQ ID NO: 27 - protein, 15 aa, Artificial Sequence
  Description of Artificial Sequence: Synthetic peptide
  MOD_RES (2)..(2): Trans-4-hydroxy-L-proline
  MOD_RES (3)..(3): 4-pentynoic acid modified residue
  MOD_RES (12)..(12): D-amino acid
  MOD_RES (14)..(14): Homoarginine
CXKVNPLCLHPDWXC

SEQ ID NO: 28 - protein, 15 aa, Artificial Sequence
  Description of Artificial Sequence: Synthetic peptide
  MOD_RES (2)..(2): Trans-4-hydroxy-L-proline
  MOD_RES (9)..(9): 4-pentynoic acid modified residue
  MOD_RES (12)..(12): D-amino acid
  MOD_RES (14)..(14): Homoarginine
CXLVNPLCKHPDWXC

SEQ ID NO: 29 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
  MOD_RES (14)..(14): Homoarginine
CXLVNPLCLKPDWXC

SEQ ID NO: 30 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
  MOD_RES (14)..(14): Homoarginine
CXKVNPLCLHPDWXC

SEQ ID NO: 31 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
  MOD_RES (14)..(14): Homoarginine
CXLVNPLCKHPDWXC

SEQ ID NO: 32 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
  MOD_RES (14)..(14): Homoarginine
CXLVNPLCLHPEWXC

SEQ ID NO: 33 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
  MOD_RES (14)..(14): Homoarginine
CXLVNPLCLEPDWXC

SEQ ID NO: 34 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
CXLVNPLCLHPDWTC

SEQ ID NO: 35 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
CXLVNPLCLEPDWTC

SEQ ID NO: 36 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
CXLVNPLCLEPAWTC

SEQ ID NO: 37 - protein, 15 aa, Artificial Sequence
  Description of Artificial Sequence: Synthetic peptide
  MOD_RES (2)..(2): Trans-4-hydroxy-L-proline
  MOD_RES (10)..(10): 3,3-diphenylalanine
  MOD_RES (12)..(12): D-amino acid
CXLVNPLCLXPDWTC

SEQ ID NO: 38 - protein, 15 aa, Artificial Sequence
  Description of Artificial Sequence: Synthetic peptide
  MOD_RES (2)..(2): Trans-4-hydroxy-L-proline
  MOD_RES (3)..(3): Beta-cyclobutylalanine
  MOD_RES (12)..(12): D-amino acid
  MOD_RES (14)..(14): Homoarginine
CXXVNPLCLHPDWXC

SEQ ID NO: 39 - protein, 15 aa, Artificial Sequence
  Description of Artificial Sequence: Synthetic peptide
  MOD_RES (2)..(2): Trans-4-hydroxy-L-proline
  MOD_RES (3)..(3): Beta-cyclobutylalanine
  MOD_RES (12)..(12): D-amino acid
CXXVNPLCLEPDWTC

SEQ ID NO: 40 - protein, 15 aa, Artificial Sequence
  Description of Artificial Sequence: Synthetic peptide
  MOD_RES (2)..(2): Trans-4-hydroxy-L-proline
  MOD_RES (3)..(3): Beta-cyclobutylalanine
  MOD_RES (10)..(10): 3,3-diphenylalanine
  MOD_RES (12)..(12): D-amino acid
CXXVNPLCLXPDWTC

SEQ ID NO: 41 - protein, 15 aa, Artificial Sequence
  Description of Artificial Sequence: Synthetic peptide
  MOD_RES (2)..(2): Trans-4-hydroxy-L-proline
  MOD_RES (10)..(10): 3,3-diphenylalanine
  MOD_RES (12)..(12): D-amino acid
  MOD_RES (14)..(14): Homoarginine
CXLVNPLCLXPDWXC

SEQ ID NO: 42 - protein, 15 aa, Artificial Sequence
  Description of Artificial Sequence: Synthetic peptide
  MOD_RES (2)..(2): Trans-4-hydroxy-L-proline
  MOD_RES (12)..(12): D-1-naphthylalanine
  MOD_RES (14)..(14): Homoarginine
CXLVNPLCLHPAWXC

SEQ ID NO: 43 - protein, 15 aa, Artificial Sequence
  Description of Artificial Sequence: Synthetic peptide
  MOD_RES (2)..(2): Trans-4-hydroxy-L-proline
  MOD_RES (10)..(10): 1-naphthylalanine
  MOD_RES (12)..(12): D-amino acid
  MOD_RES (14)..(14): Homoarginine
CXLVNPLCLAPDWXC

SEQ ID NO: 44 - protein, 15 aa, Artificial Sequence
  Description of Artificial Sequence: Synthetic peptide
  MOD_RES (2)..(2): Trans-4-hydroxy-L-proline
  MOD_RES (12)..(12): D-1-naphthylalanine
CXLVNPLCLEPAWTC

SEQ ID NO: 45 - protein, 15 aa, Artificial Sequence
  Description of Artificial Sequence: Synthetic peptide
  MOD_RES (2)..(2): Trans-4-hydroxy-L-proline
  MOD_RES (10)..(10): 1-naphthylalanine
  MOD_RES (12)..(12): D-amino acid
CXLVNPLCLAPDWTC

SEQ ID NO: 46 - protein, 15 aa, Artificial Sequence
  Description of Artificial Sequence: Synthetic peptide
  MOD_RES (2)..(2): Trans-4-hydroxy-L-proline
  MOD_RES (3)..(3): Beta-cyclobutylalanine
  MOD_RES (12)..(12): D-amino acid
CXXVNPLCLEPAWTC

SEQ ID NO: 47 - protein, 15 aa, Artificial Sequence
  Description of Artificial Sequence: Synthetic peptide
  MOD_RES (2)..(2): Trans-4-hydroxy-L-proline
  MOD_RES (3)..(3): homoglutamic acid
  MOD_RES (12)..(12): D-amino acid
  MOD_RES (14)..(14): Homoarginine
CXXVNPLCLHPDWXC

SEQ ID NO: 48 - protein, 15 aa, Artificial Sequence
  Description of Artificial Sequence: Synthetic peptide
  MOD_RES (2)..(2): Trans-4-hydroxy-L-proline
  MOD_RES (9)..(9): homoglutamic acid
  MOD_RES (12)..(12): D-amino acid
  MOD_RES (14)..(14): Homoarginine
CXLVNPLCXHPDWXC

SEQ ID NO: 49 - protein, 15 aa, Artificial Sequence
  Description of Artificial Sequence: Synthetic peptide
  MOD_RES (2)..(2): Trans-4-hydroxy-L-proline
  MOD_RES (10)..(10): homoglutamic acid
  MOD_RES (12)..(12): D-amino acid
  MOD_RES (14)..(14): Homoarginine
CXLVNPLCLXPDWXC

SEQ ID NO: 50 - protein, 15 aa, Artificial Sequence
  Description of Artificial Sequence: Synthetic peptide
  MOD_RES (2)..(2): Trans-4-hydroxy-L-proline
  MOD_RES (12)..(12): D-Norleucine
  MOD_RES (14)..(14): Homoarginine
CXLVNPLCLHPXWXC

SEQ ID NO: 51 - protein, 15 aa, Artificial Sequence
  Description of Artificial Sequence: Synthetic peptide
  MOD_RES (2)..(2): Trans-4-hydroxy-L-proline
  MOD_RES (10)..(10): Norleucine
  MOD_RES (12)..(12): D-amino acid
  MOD_RES (14)..(14): Homoarginine
CXLVNPLCLXPDWXC

SEQ ID NO: 52 - protein, 15 aa, Artificial Sequence
  Description of Artificial Sequence: Synthetic peptide
CSAGWLTMCQKLHLC

SEQ ID NO: 53 - protein, 15 aa, Artificial Sequence
  Description of Artificial Sequence: Synthetic peptide
  MOD_RES (11)..(11): 4-pentynoic acid modified residue
CSAGWLTMCQKLHLC

SEQ ID NO: 54 - protein, 15 aa, Artificial Sequence
  Description of Artificial Sequence: Synthetic peptide
  MOD_RES (11)..(11): Acetylated lysine
CSKGWLTMCQKLHLC

SEQ ID NO: 55 - protein, 15 aa, Artificial Sequence
  Description of Artificial Sequence: Synthetic peptide
  MOD_RES (11)..(11): Acetylated lysine
CSAGWLTKCQKLHLC

SEQ ID NO: 56 - protein, 15 aa, Artificial Sequence
  Description of Artificial Sequence: Synthetic peptide
  MOD_RES (11)..(11): Acetylated lysine
CSAGWLTMCKKLHLC

SEQ ID NO: 57 - protein, 15 aa, Artificial Sequence
  Description of Artificial Sequence: Synthetic peptide
  MOD_RES (11)..(11): Acetylated lysine
CSAGWLTMCQKLKLC

SEQ ID NO: 58 - protein, 15 aa, Artificial Sequence
  Description of Artificial Sequence: Synthetic peptide
  MOD_RES (11)..(11): Homoarginine
CSAGWLTMCQXLHLC

SEQ ID NO: 59 - protein, 15 aa, Artificial Sequence
  Description of Artificial Sequence: Synthetic peptide
  MOD_RES (10)..(10): Homoarginine
CSAGWLTMCXQLNLC

SEQ ID NO: 60 - protein, 15 aa, Artificial Sequence
  Description of Artificial Sequence: Synthetic peptide
  MOD_RES (2)..(2): t-butyl-alanine
  MOD_RES (5)..(5): D-amino acid
  MOD_RES (14)..(14): Norleucine
CAPEKPYCFADPYXC

SEQ ID NO: 61 - protein, 15 aa, Artificial Sequence
  Description of Artificial Sequence: Synthetic peptide
  MOD_RES (5)..(5): 4-pentynoic acid modified D-amino acid
  MOD_RES (14)..(14): Norleucine
CIEEKQYCFADPYXC

SEQ ID NO: 62 - protein, 15 aa, Artificial Sequence
  Description of Artificial Sequence: Synthetic peptide
  MOD_RES (2)..(2): t-butyl-alanine
  MOD_RES (5)..(5): D-amino acid
  MOD_RES (14)..(14): Norleucine
CAEEKPYCFADPYXC

SEQ ID NO: 63 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine
CAPEKPYCFADPYXC

SEQ ID NO: 64 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine
CAEEKPYCFADPYXC

SEQ ID NO: 65 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine
CAPEKPYCFANPYXC

SEQ ID NO: 66 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine
CAPEKPYCFAEPYXC

SEQ ID NO: 67 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (11)..(11): Alpha-L-aminoadipic acid
  MOD_RES (14)..(14): Norleucine
CAPEKPYCFAXPYXC

SEQ ID NO: 68 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine
CAPEKPYCFAQPYXC

SEQ ID NO: 69 - protein, 14 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine modified with Cysteamine
CAPEKPYCFADPYX

SEQ ID NO: 70 - protein, 14 aa, Artificial Sequence
  Description of Artificial Sequence: Synthetic peptide
  MOD_RES (1)..(1): t-butyl-alanine modified with 3-mercaptopropionic acid
  MOD_RES (4)..(4): 4-pentynoic acid modified D-amino acid
  MOD_RES (13)..(13): Norleucine
APEKPYCFADPYXC

SEQ ID NO: 71 - protein, 13 aa, Artificial Sequence
  Description of Artificial Sequence: Synthetic peptide
  MOD_RES (1)..(1): t-butyl-alanine modified with 3-mercaptopropionic acid
  MOD_RES (4)..(4): 4-pentynoic acid modified D-amino acid
  MOD_RES (13)..(13): Norleucine modified with cysteamine
APEKPYCFADPYX

SEQ ID NO: 72 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine
CAPEKPYCFADPYXC

SEQ ID NO: 73 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine
CAPEKPYCFADPYXC

SEQ ID NO: 74 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine
CAPEKPYCFADPYXC

SEQ ID NO: 75 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (12)..(12): N-methyl-alanine
  MOD_RES (14)..(14): Norleucine
CAPEKPYCFADAYXC

SEQ ID NO: 76 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (12)..(12): N-methyl-D-alanine
  MOD_RES (14)..(14): Norleucine
CAPEKPYCFADAYXC

SEQ ID NO: 77 - protein, 15 aa, Artificial Sequence
  Description of Artificial Sequence: Synthetic peptide
  MOD_RES (2)..(2): t-butyl-alanine
  MOD_RES (4)..(4): 4-pentynoic acid modified residue
  MOD_RES (5)..(5): D-amino acid
  MOD_RES (14)..(14): Norleucine
CAPKAPYCFADPYXC

SEQ ID NO: 78 - protein, 15 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine
CAPEKPYCFADPYXC

SEQ ID NO: 79 - protein, 15 aa, Artificial Sequence
  Description of Artificial Sequence: Synthetic peptide
  MOD_RES (2)..(2): t-butyl-alanine
  MOD_RES (5)..(5): Methylated D-amino acid modified with 4-pentynoic acid
  MOD_RES (14)..(14): Norleucine
CAPEKPYCFADPYXC

SEQ ID NO: 80 - protein, 15 aa, Artificial Sequence
  Description of Artificial Sequence: Synthetic peptide
  MOD_RES (2)..(2): t-butyl-alanine
  MOD_RES (5)..(5): Methylated D-amino acid modified with 4-pentynoic acid
  MOD_RES (14)..(14): Norleucine
CAPEKPYCFADPYXC

SEQ ID NO: 81 - protein, 14 aa, Artificial Sequence
  Description of Artificial Sequence: Synthetic peptide
  MOD_RES (1)..(1): t-butyl-alanine modified with 3-mercaptopropionic acid
  MOD_RES (4)..(4): D-amino acid
  MOD_RES (13)..(13): Norleucine
AEEKPYCFADPYXC

SEQ ID NO: 82 - protein, 15 aa, Artificial Sequence
  Description of Artificial Sequence: Synthetic peptide
  MOD_RES (2)..(2): Homoarginine
CXDWCHWTFSHGHPC

SEQ ID NO: 83-153 - skipped (71 entries)

SEQ ID NO: 154 - protein, 14 aa, Artificial Sequence
  Description of Artificial Sequence: Synthetic peptide
  MOD_RES (1)..(1): Trans-4-hydroxy-L-proline modified with 3-mercaptopropionic acid
  MOD_RES (9)..(9): 3,3-diphenylalanine
  MOD_RES (11)..(11): D-amino acid
XLVNPLCLXPDWTC

SEQ ID NO: 155 - protein, 14 aa, Artificial Sequence
  Description of Artificial Sequence: Synthetic peptide
  MOD_RES (2)..(2): Trans-4-hydroxy-L-proline
  MOD_RES (12)..(12): D-amino acid
  MOD_RES (14)..(14): Homoarginine modified with cysteamine
CXLVNPLCLHPDWX

SEQ ID NO: 156 - protein, 15 aa, Artificial Sequence
  Description of Artificial Sequence: Synthetic peptide
  MOD_RES (2)..(2): Trans-4-hydroxy-L-proline
  MOD_RES (10)..(10): N3-methyl-L-histidine
  MOD_RES (12)..(12): D-amino acid
  MOD_RES (14)..(14): Homoarginine
CXLVNPLCLXPDWXC

SEQ ID NO: 157 - protein, 15 aa, Artificial Sequence
  Description of Artificial Sequence: Synthetic peptide
  MOD_RES (2)..(2): Trans-4-hydroxy-L-proline
  MOD_RES (10)..(10): N1-methyl-L-histidine
  MOD_RES (12)..(12): D-amino acid
  MOD_RES (14)..(14): Homoarginine
CXLVNPLCLXPDWXC

SEQ ID NO: 158 - protein, 15 aa, Artificial Sequence
  Description of Artificial Sequence: Synthetic peptide
  MOD_RES (2)..(2): Trans-4-hydroxy-L-proline
  MOD_RES (10)..(10): Beta-(4-thiazolyl)-alanine
  MOD_RES (12)..(12): D-amino acid
  MOD_RES (14)..(14): Homoarginine
CXLVNPLCLXPDWXC

SEQ ID NO: 159 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
  MOD_RES (14)..(14): Homoarginine
CXLVNPLCLFPDWXC

SEQ ID NO: 160 - protein, 15 aa, Artificial Sequence
  Description of Artificial Sequence: Synthetic peptide
  MOD_RES (2)..(2): Trans-4-hydroxy-L-proline
  MOD_RES (10)..(10): 2-Thienyl-alanine
  MOD_RES (12)..(12): D-amino acid
  MOD_RES (14)..(14): Homoarginine
CXLVNPLCLXPDWXC

SEQ ID NO: 161 - protein, 15 aa, Artificial Sequence
  Description of Artificial Sequence: Synthetic peptide
  MOD_RES (2)..(2): Trans-4-hydroxy-L-proline
  MOD_RES (10)..(10): 3-Thienyl-alanine
  MOD_RES (12)..(12): D-amino acid
  MOD_RES (14)..(14): Homoarginine
CXLVNPLCLXPDWXC

SEQ ID NO: 162 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
  MOD_RES (14)..(14): Homoarginine
CXLVNPLCLNPDWXC

SEQ ID NO: 163 - protein, 15 aa, Artificial Sequence
  Description of Artificial Sequence: Synthetic peptide
  MOD_RES (2)..(2): Trans-4-hydroxy-L-proline
  MOD_RES (12)..(12): D-amino acid
  MOD_RES (14)..(14): Homoarginine
CXLVNPLCLQPDWXC

SEQ ID NO: 164 - protein, 15 aa, Artificial Sequence
  Description of Artificial Sequence: Synthetic peptide
  MOD_RES (2)..(2): Trans-4-hydroxy-L-proline
  MOD_RES (10)..(10): 4-pentynoic acid-Palmitoyl-Glu-LysN3 modified residue
  MOD_RES (12)..(12): D-amino acid
  MOD_RES (14)..(14): Homoarginine
CXLVNPLCLKPDWXC

SEQ ID NO: 165 - protein, 26 aa, Artificial Sequence
  Description of Artificial Sequence: Synthetic peptide
  MOD_RES (1)..(1): 4-pentynoic acid modified beta-alanine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine
  MOD_RES (14)..(14): 1-naphthylalanine
  MOD_RES (15)..(15): D-amino acid
  MOD_RES (18)..(18): Homoarginine
  MOD_RES (24)..(24): Trans-4-hydroxy-L-proline
AXXXXXXXXXXCPADCMXDWSTPXWC

SEQ ID NO: 166 - protein, 15 aa, Artificial Sequence
  Description of Artificial Sequence: Synthetic peptide
  MOD_RES (1)..(1): 4-pentynoic acid modified residue
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPADCMXDWSTPXWC

SEQ ID NO: 167 - protein, 16 aa, Artificial Sequence
  Description of Artificial Sequence: Synthetic peptide
  MOD_RES (1)..(1): 4-pentynoic acid modified beta-alanine
  MOD_RES (4)..(4): 1-naphthylalanine
  MOD_RES (5)..(5): D-amino acid
  MOD_RES (8)..(8): Homoarginine
  MOD_RES (14)..(14): Trans-4-hydroxy-L-proline
ACPADCMXDWSTPXWC

SEQ ID NO: 168 - protein, 26 aa, Artificial Sequence
  Description of Artificial Sequence: Synthetic peptide
  MOD_RES (1)..(1): 4-pentynoic acid modified beta-alanine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine
  MOD_RES (14)..(14): 1-naphthylalanine
  MOD_RES (15)..(15): D-amino acid
  MOD_RES (18)..(18): Homoarginine
  MOD_RES (20)..(20): D-amino acid
  MOD_RES (24)..(24): Trans-4-hydroxy-L-proline
  MOD_RES (25)..(25): D-amino acid
AXXXXXXXXXXCPADCMXDWSTPXWC

SEQ ID NO: 169 - protein, 26 aa, Artificial Sequence
  Description of Artificial Sequence: Synthetic peptide
  N-term Ac
  MOD_RES (3)..(3): 1-naphthylalanine
  MOD_RES (4)..(4): D-amino acid
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine
  MOD_RES (12)..(12): Sarcosine
  MOD_RES (13)..(13): Sarcosine
  MOD_RES (14)..(14): Sarcosine
  MOD_RES (15)..(15): Beta-alanine
  MOD_RES (18)..(18): Homoarginine
  MOD_RES (24)..(24): Trans-4-hydroxy-L-proline
CPAKXXXXXXXXXXACMXDWSTPXWC

SEQ ID NO: 170 - protein, 16 aa, Artificial Sequence
  Description of Artificial Sequence: Synthetic peptide
  MOD_RES (1)..(1): 4-pentynoic acid modified beta-alanine
  MOD_RES (4)..(4): 1-naphthylalanine
  MOD_RES (5)..(5): D-amino acid
  MOD_RES (8)..(8): Homoarginine
  MOD_RES (14)..(14): Trans-4-hydroxy-L-proline
ACPAKCMXDWSTPXWC

SEQ ID NO: 171 - protein, 17 aa, Artificial Sequence
  Description of Artificial Sequence: Synthetic peptide
  MOD_RES (1)..(1): Palmitoyl modified gamma-glutamic acid
  MOD_RES (2)..(2): Gamma-glutamic acid
  MOD_RES (5)..(5): 1-naphthylalanine
  MOD_RES (6)..(6): D-amino acid
  MOD_RES (9)..(9): Homoarginine
  MOD_RES (15)..(15): Trans-4-hydroxy-L-proline
XXCPAKCMXDWSTPXWC

SEQ ID NO: 172 - protein, 15 aa, Artificial Sequence
  Description of Artificial Sequence: Synthetic peptide
  N-term Ac
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPAKCMXDWSTPXWC

SEQ ID NO: 173 - protein, 14 aa, Artificial Sequence
  Description of Artificial Sequence: Synthetic peptide
  N-term Ac
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
  MOD_RES (14)..(14): Cysteamine modified residue
CPAKCMXDWSTPXW

SEQ ID NO: 174 - protein, 15 aa, Artificial Sequence
  Description of Artificial Sequence: Synthetic peptide
  N-term Ac
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPAKCMXHWSTPXWC

SEQ ID NO: 175 - protein, 15 aa, Artificial Sequence
  Description of Artificial Sequence: Synthetic peptide
  N-term Ac
  MOD_RES (3)..(3): 1-naphthylalanine
  MOD_RES (4)..(4): D-amino acid
  MOD_RES (7)..(7): Homoarginine
  MOD_RES (13)..(13): Trans-4-hydroxy-L-proline
CPAKCMXEWSTPXWC

SEQ ID NO: 176 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLHPDWXC

SEQ ID NO: 177 - protein, 29 aa, Artificial Sequence
  Description of Artificial Sequence: Synthetic peptide
  MOD_RES (1)..(1): Beta-alanine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine
  MOD_RES (13)..(13): Homoarginine
  MOD_RES (16)..(16): Trans-4-hydroxy-L-proline
  MOD_RES (26)..(26): D-amino acid
  MOD_RES (28)..(28): Homoarginine
AXXXXXXXXXXAXDCXLVNPLCLHPDWXC

SEQ ID NO: 178 - protein, 18 aa, Artificial Sequence
  Description of Artificial Sequence: Synthetic peptide
  MOD_RES (1)..(1): 4-pentynoic acid modified residue
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLHPDWXC

SEQ ID NO: 179 - protein, 19 aa, Artificial Sequence
  Description of Artificial Sequence: Synthetic peptide
  N-term Ac
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
  MOD_RES (19)..(19): 4-pentynoic acid modified residue
AXDCXLVNPLCLHPDWXCK

SEQ ID NO: 180 - protein, 19 aa, Artificial Sequence
  Description of Artificial Sequence: Synthetic peptide
  N-term Ac
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLHPDWXCK

SEQ ID NO: 181 - protein, 18 aa, Artificial Sequence
  Description of Artificial Sequence: Synthetic peptide
  MOD_RES (1)..(1): N-methyl-alanine
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLHPDWXC

SEQ ID NO: 182 - protein, 19 aa, Artificial Sequence
  Description of Artificial Sequence: Synthetic peptide
  N-term Ac
  MOD_RES (2)..(2): Trans-4-hydroxy-L-proline
  MOD_RES (12)..(12): D-amino acid
  MOD_RES (14)..(14): Homoarginine
  MOD_RES (17)..(17): D-amino acid
  MOD_RES (19)..(19): D-amino acid
CXLVNPLCLHPDWXCLHGK

SEQ ID NO: 183 - protein, 29 aa, Artificial Sequence
  Description of Artificial Sequence: Synthetic peptide
  MOD_RES (1)..(1): 4-pentynoic acid modified beta-alanine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine
  MOD_RES (28)..(28): Homoarginine
AXXXXXXXXXXVGPCLWDPTPCANLHLXC

SEQ ID NO: 184 - protein, 18 aa, Artificial Sequence
  Description of Artificial Sequence: Synthetic peptide
  N-term Ac
  MOD_RES (2)..(2): Homoarginine
  MOD_RES (5)..(5): Trans-4-hydroxy-L-proline
  MOD_RES (13)..(13): 4-pentynoic acid modified residue
  MOD_RES (15)..(15): D-amino acid
  MOD_RES (17)..(17): Homoarginine
AXDCXLVNPLCLKPDWXC

SEQ ID NO: 185 - protein, 18 aa, Artificial Sequence
  Description of Artificial Sequence: Synthetic peptide
  N-term Ac
  MOD_RES (2)..(2): Homoarginine
  MOD_RES (5)..(5): Trans-4-hydroxy-L-proline
  MOD_RES (6)..(6): 4-pentynoic acid modified residue
  MOD_RES (15)..(15): D-amino acid
  MOD_RES (17)..(17): Homoarginine
AXDCXKVNPLCLHPDWXC

SEQ ID NO: 186 - protein, 18 aa, Artificial Sequence
  Description of Artificial Sequence: Synthetic peptide
  N-term Ac
  MOD_RES (2)..(2): Homoarginine
  MOD_RES (5)..(5): Trans-4-hydroxy-L-proline
  MOD_RES (12)..(12): 4-pentynoic acid modified residue
  MOD_RES (15)..(15): D-amino acid
  MOD_RES (17)..(17): Homoarginine
AXDCXLVNPLCKHPDWXC

SEQ ID NO: 187 - protein, 18 aa, Artificial Sequence
  Description of Artificial Sequence: Synthetic peptide
  N-term Ac
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLKPDWXC

SEQ ID NO: 188 - protein, 18 aa, Artificial Sequence
  Description of Artificial Sequence: Synthetic peptide
  MOD_RES (1)..(1): 4-pentynoic acid-Palmitoyl-Glu-LysN3 modified residue
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLKPDWXC

SEQ ID NO: 189 - protein, 18 aa, Artificial Sequence
  Description of Artificial Sequence: Synthetic peptide
  N-term Ac
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXKVNPLCLHPDWXC

SEQ ID NO: 190 - protein, 18 aa, Artificial Sequence
  Description of Artificial Sequence: Synthetic peptide
  N-term Ac
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCKHPDWXC

SEQ ID NO: 191 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLHPEWXC

SEQ ID NO: 192 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXECXLVNPLCLHPEWXC

SEQ ID NO: 193 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLEPDWXC

SEQ ID NO: 194 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
AXDCXLVNPLCLHPDWTC

SEQ ID NO: 195 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
AXDCXLVNPLCLEPDWTC

SEQ ID NO: 196 - protein, 19 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
  MOD_RES (19)..(19): D-amino acid
AXDCXLVNPLCLEPDWTCA

SEQ ID NO: 197 - protein, 16 aa, Artificial Sequence
  Description of Artificial Sequence: Synthetic peptide
  MOD_RES (2)..(2): Trans-4-hydroxy-L-proline
  MOD_RES (12)..(12): D-amino acid
  MOD_RES (16)..(16): D-amino acid
CXLVNPLCLEPDWTCA

SEQ ID NO: 198 - protein, 18 aa, Artificial Sequence
  Description of Artificial Sequence: Synthetic peptide
  MOD_RES (1)..(1): N-methyl-alanine
  MOD_RES (2)..(2): Homoarginine
  MOD_RES (5)..(5): Trans-4-hydroxy-L-proline
  MOD_RES (15)..(15): D-amino acid
AXDCXLVNPLCLEPDWTC

SEQ ID NO: 199 - protein, 18 aa, Artificial Sequence
  Description of Artificial Sequence: Synthetic peptide
  MOD_RES (1)..(1): D-amino acid
  MOD_RES (5)..(5): Trans-4-hydroxy-L-proline
  MOD_RES (15)..(15): D-amino acid
AEDCXLVNPLCLEPDWTC

SEQ ID NO: 200 - protein, 18 aa, Artificial Sequence
  Description of Artificial Sequence: Synthetic peptide
  MOD_RES (1)..(1): D-amino acid
  MOD_RES (2)..(2): D-amino acid
  MOD_RES (5)..(5): Trans-4-hydroxy-L-proline
  MOD_RES (15)..(15): D-amino acid
AADCXLVNPLCLEPDWTC

SEQ ID NO: 201 - protein, 17 aa, Artificial Sequence
  Description of Artificial Sequence: Synthetic peptide
  MOD_RES (4)..(4): Trans-4-hydroxy-L-proline
  MOD_RES (14)..(14): D-amino acid
ADCXLVNPLCLEPDWTC

SEQ ID NO: 202 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
AXDCXLVNPLCLEPAWTC

SEQ ID NO: 203 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (13)..(13): 3,3-diphenylalanine
  MOD_RES (15)..(15): D-amino acid
AXDCXLVNPLCLXPDWTC

SEQ ID NO: 204 - protein, 16 aa, Artificial Sequence
  Description of Artificial Sequence: Synthetic peptide
  N-term Ac
  MOD_RES (2)..(2): Trans-4-hydroxy-L-proline
  MOD_RES (10)..(10): 3,3-diphenylalanine
  MOD_RES (12)..(12): D-amino acid
  MOD_RES (16)..(16): D-amino acid
CXLVNPLCLXPDWTCK

SEQ ID NO: 205 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (6)..(6): Beta-cyclobutylalanine
  MOD_RES (15)..(15): D-amino acid
  MOD_RES (17)..(17): Homoarginine
AXDCXXVNPLCLHPDWXC

SEQ ID NO: 206 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (6)..(6): Beta-cyclobutylalanine
  MOD_RES (15)..(15): D-amino acid
AXDCXXVNPLCLEPDWTC

SEQ ID NO: 207 - protein, 19 aa, Artificial Sequence
  Description of Artificial Sequence: Synthetic peptide
  MOD_RES (1)..(1): D-amino acid
  MOD_RES (2)..(2): Homoarginine
  MOD_RES (5)..(5): Trans-4-hydroxy-L-proline
  MOD_RES (6)..(6): Beta-cyclobutylalanine
  MOD_RES (15)..(15): D-amino acid
  MOD_RES (19)..(19): D-amino acid
AXDCXXVNPLCLEPDWTCA

SEQ ID NO: 208 - protein, 16 aa, Artificial Sequence
  Description of Artificial Sequence: Synthetic peptide
  MOD_RES (2)..(2): Trans-4-hydroxy-L-proline
  MOD_RES (3)..(3): Beta-cyclobutylalanine
  MOD_RES (10)..(10): 3,3-diphenylalanine
  MOD_RES (12)..(12): D-amino acid
  MOD_RES (16)..(16): D-amino acid
CXXVNPLCLXPDWTCA

SEQ ID NO: 209 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (13)..(13): 3,3-diphenylalanine
  MOD_RES (15)..(15): D-amino acid
  MOD_RES (17)..(17): Homoarginine
AXDCXLVNPLCLXPDWXC

SEQ ID NO: 210 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-1-naphthylalanine
  MOD_RES (17)..(17): Homoarginine
AXDCXLVNPLCLHPAWXC

SEQ ID NO: 211 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (13)..(13): 1-naphthylalanine
  MOD_RES (15)..(15): D-amino acid
  MOD_RES (17)..(17): Homoarginine
AXDCXLVNPLCLAPDWXC

SEQ ID NO: 212 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-1-naphthylalanine
AXDCXLVNPLCLEPAWTC

SEQ ID NO: 213 - protein, 19 aa, Artificial Sequence
  Description of Artificial Sequence: Synthetic peptide
  MOD_RES (1)..(1): D-amino acid
  MOD_RES (2)..(2): Homoarginine
  MOD_RES (5)..(5): Trans-4-hydroxy-L-proline
  MOD_RES (6)..(6): Beta-cyclobutylalanine
  MOD_RES (15)..(15): D-amino acid
  MOD_RES (19)..(19): D-amino acid
AXDCXXVNPLCLEPAWTCA

SEQ ID NO: 214 - protein, 19 aa, Artificial Sequence
  Description of Artificial Sequence: Synthetic peptide
  MOD_RES (1)..(1): D-1-naphthylalanine
  MOD_RES (2)..(2): Homoarginine
  MOD_RES (5)..(5): Trans-4-hydroxy-L-proline
  MOD_RES (6)..(6): Beta-cyclobutylalanine
  MOD_RES (15)..(15): D-amino acid
  MOD_RES (19)..(19): D-amino acid
AXDCXXVNPLCLEPAWTCA

SEQ ID NO: 215 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (6)..(6): homoglutamic acid
  MOD_RES (15)..(15): D-amino acid
  MOD_RES (17)..(17): Homoarginine
AXDCXXVNPLCLHPDWXC

SEQ ID NO: 216 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (12)..(12): homoglutamic acid
  MOD_RES (15)..(15): D-amino acid
  MOD_RES (17)..(17): Homoarginine
AXDCXLVNPLCXHPDWXC

SEQ ID NO: 217 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (13)..(13): homoglutamic acid
  MOD_RES (15)..(15): D-amino acid
  MOD_RES (17)..(17): Homoarginine
AXDCXLVNPLCLXPDWXC

SEQ ID NO: 218 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-Norleucine
  MOD_RES (17)..(17): Homoarginine
AXDCXLVNPLCLHPXWXC

SEQ ID NO: 219 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (13)..(13): Norleucine
  MOD_RES (15)..(15): D-amino acid
  MOD_RES (17)..(17): Homoarginine
AXDCXLVNPLCLXPDWXC

SEQ ID NO: 220 - protein, 15 aa, Artificial Sequence
  Description of Artificial Sequence: Synthetic peptide
  MOD_RES (1)..(1): Trans-4-hydroxy-L-proline modified with 3-mercaptopropionic acid
  MOD_RES (9)..(9): 3,3-diphenylalanine
  MOD_RES (11)..(11): D-amino acid
  MOD_RES (15)..(15): D-amino acid
XLVNPLCLXPDWTCK

SEQ ID NO: 221 - protein, 17 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine modified with cysteamine
AXDCXLVNPLCLHPDWX

SEQ ID NO: 222 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (13)..(13): N3-methyl-L-histidine
  MOD_RES (15)..(15): D-amino acid
  MOD_RES (17)..(17): Homoarginine
AXDCXLVNPLCLXPDWXC

SEQ ID NO: 223 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (13)..(13): N1-methyl-L-histidine
  MOD_RES (15)..(15): D-amino acid
  MOD_RES (17)..(17): Homoarginine
AXDCXLVNPLCLXPDWXC

SEQ ID NO: 224 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (13)..(13): Beta-(4-thiazolyl)-alanine
  MOD_RES (15)..(15): D-amino acid
  MOD_RES (17)..(17): Homoarginine
AXDCXLVNPLCLXPDWXC

SEQ ID NO: 225 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLFPDWXC

SEQ ID NO: 226 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (13)..(13): 2-Thienyl-alanine
  MOD_RES (15)..(15): D-amino acid
  MOD_RES (17)..(17): Homoarginine
AXDCXLVNPLCLXPDWXC

SEQ ID NO: 227 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (13)..(13): 3-Thienyl-alanine
  MOD_RES (15)..(15): D-amino acid
  MOD_RES (17)..(17): Homoarginine
AXDCXLVNPLCLXPDWXC

SEQ ID NO: 228 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLNPDWXC

SEQ ID NO: 229 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLQPDWXC

SEQ ID NO: 230 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (13)..(13): 4-pentynoic acid-Palmitoyl-Glu-LysN3 modified residue
  MOD_RES (15)..(15): D-amino acid
  MOD_RES (17)..(17): Homoarginine
AXDCXLVNPLCLKPDWXC

SEQ ID NO: 231 - protein, 18 aa, Artificial Sequence
  Description of Artificial Sequence: Synthetic peptide
  MOD_RES (2)..(2): Homoarginine
  MOD_RES (5)..(5): Trans-4-hydroxy-L-proline
  MOD_RES (15)..(15): D-amino acid
  MOD_RES (17)..(17): Homoarginine
AXDCXLVNPLCLHPDWXC

SEQ ID NO: 232 - protein, 29 aa, Artificial Sequence
  Description of Artificial Sequence: Synthetic peptide
  MOD_RES (1)..(1): 4-pentynoic acid modified beta-alanine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine
  MOD_RES (13)..(13): Homoarginine
  MOD_RES (16)..(16): Trans-4-hydroxy-L-proline
  MOD_RES (26)..(26): D-amino acid
  MOD_RES (28)..(28): Homoarginine
AXXXXXXXXXXAXDCXLVNPLCLHPDWXC

SEQ ID NO: 233 - protein, 29 aa, Artificial Sequence
  Description of Artificial Sequence: Synthetic peptide
  MOD_RES (1)..(1): 4-pentynoic acid modified beta-alanine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine
AXXXXXXXXXXSDKCSAGWLTMCQKLHLC

SEQ ID NO: 234 - protein, 20 aa, Artificial Sequence
  Description of Artificial Sequence: Synthetic peptide
  N-term Ac
  MOD_RES (2)..(2): Homoarginine
DXCSAGWLTMCQKLHLCPSH

SEQ ID NO: 235 - protein, 18 aa, Artificial Sequence
  Description of Artificial Sequence: Synthetic peptide
  N-term Ac
  MOD_RES (14)..(14): 4-pentynoic acid modified residue
SDKCSAGWLTMCQKLHLC

SEQ ID NO: 236 - protein, 21 aa, Artificial Sequence
  Description of Artificial Sequence: Synthetic peptide
  N-term Ac
  MOD_RES (14)..(14): 4-pentynoic acid modified residue
SDKCSAGWLTMCQKLHLCPSH

SEQ ID NO: 237 - protein, 20 aa, Artificial Sequence
  Description of Artificial Sequence: Synthetic peptide
  N-term Ac
  MOD_RES (2)..(2): Homoarginine
  MOD_RES (13)..(13): Acetylated lysine
DXCSKGWLTMCQKLHLCPSH

SEQ ID NO: 238 - protein, 20 aa, Artificial Sequence
  Description of Artificial Sequence: Synthetic peptide
  N-term Ac
  MOD_RES (2)..(2): Homoarginine
  MOD_RES (13)..(13): Acetylated lysine
DXCSAGWLTKCQKLHLCPSH

SEQ ID NO: 239 - protein, 20 aa, Artificial Sequence
  Description of Artificial Sequence: Synthetic peptide
  N-term Ac
  MOD_RES (2)..(2): Homoarginine
  MOD_RES (13)..(13): Acetylated lysine
DXCSAGWLTMCKKLHLCPSH

SEQ ID NO: 240 - protein, 20 aa, Artificial Sequence
  Description of Artificial Sequence: Synthetic peptide
  N-term Ac
  MOD_RES (2)..(2): Homoarginine
  MOD_RES (13)..(13): Acetylated lysine
DXCSAGWLTMCQKLKLCPSH

SEQ ID NO: 241 - protein, 22 aa, Artificial Sequence
  Description of Artificial Sequence: Synthetic peptide
  N-term Ac
  MOD_RES (3)..(3): Homoarginine
  MOD_RES (14)..(14): Homoarginine
SDXCSAGWLTMCQXLHLCPSHK

SEQ ID NO: 242 - protein, 22 aa, Artificial Sequence
  Description of Artificial Sequence: Synthetic peptide
  N-term Ac
  MOD_RES (3)..(3): Homoarginine
  MOD_RES (13)..(13): Homoarginine
SDXCSAGWLTMCXQLNLCPSHK

SEQ ID NO: 243 - protein, 26 aa, Artificial Sequence
  Description of Artificial Sequence: Synthetic peptide
  MOD_RES (1)..(1): 4-pentynoic acid modified beta-alanine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine
  MOD_RES (13)..(13): Homoarginine
AXXXXXXXXXXCXDWCHWTFSHGHPC

SEQ ID NO: 244 - protein, 29 aa, Artificial Sequence
  Description of Artificial Sequence: Synthetic peptide
  MOD_RES (19)..(19): Sarcosine
  MOD_RES (20)..(20): Sarcosine
  MOD_RES (21)..(21): Sarcosine
  MOD_RES (22)..(22): Sarcosine
  MOD_RES (23)..(23): Sarcosine
  MOD_RES (24)..(24): Sarcosine
  MOD_RES (25)..(25): Sarcosine
  MOD_RES (26)..(26): Sarcosine
  MOD_RES (27)..(27): Sarcosine
  MOD_RES (28)..(28): Sarcosine
  MOD_RES (29)..(29): 4-pentynoic acid modified residue
SDKCSAGWLTMCQKLHLCXXXXXXXXXXK

SEQ ID NO: 245 - protein, 18 aa, Artificial Sequence
  Description of Artificial Sequence: Synthetic peptide
  MOD_RES (14)..(14): 4-pentynoic acid modified residue
SDKCSAGWLTMCQKLHLC

SEQ ID NO: 246 - protein, 17 aa, Artificial Sequence
  Description of Artificial Sequence: Synthetic peptide
  N-term Ac
  MOD_RES (15)..(15): Norleucine
  MOD_RES (17)..(17): Diaminopropionic acid
ACIEEGQYCFADPYXCX

SEQ ID NO: 247 - protein, 17 aa, Artificial Sequence
  Description of Artificial Sequence: Synthetic peptide
  N-term Ac
  MOD_RES (15)..(15): Norleucine
  MOD_RES (17)..(17): Diaminopropionic acid modified with 4-pentynoic acid
ACIEEGQYCFADPYXCX

SEQ ID NO: 248 - protein, 16 aa, Artificial Sequence
  Description of Artificial Sequence: Synthetic peptide
  N-term Ac
  MOD_RES (2)..(2): t-butyl-alanine
  MOD_RES (5)..(5): D-amino acid
  MOD_RES (14)..(14): Norleucine
  MOD_RES (16)..(16): Diaminopropionic acid
CAPEAPYCFADPYXCX

SEQ ID NO: 249 - protein, 16 aa, Artificial Sequence
  Description of Artificial Sequence: Synthetic peptide
  N-term Ac
  MOD_RES (2)..(2): t-butyl-alanine
  MOD_RES (5)..(5): D-amino acid
  MOD_RES (14)..(14): Norleucine
  MOD_RES (16)..(16): Diaminopropionic acid modified with 4-pentynoic acid
CAPEAPYCFADPYXCX

SEQ ID NO: 250 - protein, 17 aa, Artificial Sequence
  Description of Artificial Sequence: Synthetic peptide
  N-term Ac
  MOD_RES (11)..(11): D-amino acid
  MOD_RES (15)..(15): Norleucine
  MOD_RES (17)..(17): Diaminopropionic acid
ACIEEGQYCFADPYXCX

SEQ ID NO: 251 - protein, 16 aa, Artificial Sequence
  Description of Artificial Sequence: Synthetic peptide
  N-term Ac
  MOD_RES (2)..(2): t-butyl-alanine
  MOD_RES (5)..(5): D-amino acid
  MOD_RES (14)..(14): Norleucine
CAPKAPYCFADPYXCA

SEQ ID NO: 252 - protein, 16 aa, Artificial Sequence
  Description of Artificial Sequence: Synthetic peptide
  N-term Ac
  MOD_RES (2)..(2): t-butyl-alanine
  MOD_RES (5)..(5): D-amino acid
  MOD_RES (14)..(14): Norleucine
CAPEKPYCFADPYXCA

SEQ ID NO: 253 - protein, 16 aa, Artificial Sequence
  Description of Artificial Sequence: Synthetic peptide
  N-term Ac
  MOD_RES (2)..(2): t-butyl-alanine
  MOD_RES (4)..(4): 4-pentynoic acid modified residue
  MOD_RES (5)..(5): D-amino acid
  MOD_RES (14)..(14): Norleucine
CAPKAPYCFADPYXCA

SEQ ID NO: 254 - protein, 16 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (14)..(14): Norleucine
CAPEKPYCFADPYXCA

SEQ ID NO: 255 - protein, 16 aa, Artificial Sequence
  Description of Artificial Sequence: Synthetic peptide
  MOD_RES (2)..(2): t-butyl-alanine
  MOD_RES (5)..(5): 4-pentynoic acid modified D-amino acid
  MOD_RES (14)..(14): Norleucine
CAPEKPYCFADPYXCA

SEQ ID NO: 256 - protein, 17 aa, Artificial Sequence
  Description of Artificial Sequence: Synthetic peptide
  N-term Ac
  MOD_RES (6)..(6): 4-pentynoic acid modified D-amino acid
  MOD_RES (15)..(15): Norleucine
ACIEEKQYCFADPYXCA

SEQ ID NO: 257 - protein, 16 aa, Artificial Sequence
  Description of Artificial Sequence: Synthetic peptide
  N-term Ac
  MOD_RES (2)..(2): t-butyl-alanine
  MOD_RES (5)..(5): D-amino acid
  MOD_RES (14)..(14): Norleucine
  MOD_RES (16)..(16): Diaminopropionic acid modified with 4-pentynoic acid
CAPEKPYCFADPYXCX

SEQ ID NO: 258 - protein, 17 aa, Artificial Sequence
  Description of Artificial Sequence: Synthetic peptide
  N-term Ac
  MOD_RES (6)..(6): 4-pentynoic acid modified D-amino acid
  MOD_RES (15)..(15): Norleucine
ACIEEKQYCFADPYXCK

SEQ ID NO: 259 - protein, 16 aa, Artificial Sequence
  Description of Artificial Sequence: Synthetic peptide
  N-term Ac
  MOD_RES (2)..(2): t-butyl-alanine
  MOD_RES (5)..(5): D-amino acid
  MOD_RES (14)..(14): Norleucine
  MOD_RES (16)..(16): Diaminopropionic acid modified with 4-pentynoic acid
CAEEKPYCFADPYXCX

SEQ ID NO: 260 - protein, 15 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (14)..(14): Norleucine
CAPEKPYCFADPYXC

SEQ ID NO: 261 - protein, 15 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (14)..(14): Norleucine
CAEEKPYCFADPYXC

SEQ ID NO: 262 - protein, 15 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (14)..(14): Norleucine
CAPEKPYCFANPYXC

SEQ ID NO: 263 - protein, 15 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (14)..(14): Norleucine
CAPEKPYCFAEPYXC

SEQ ID NO: 264 - protein, 15 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (11)..(11): Alpha-L-aminoadipic acid
  MOD_RES (14)..(14): Norleucine
CAPEKPYCFAXPYXC

SEQ ID NO: 265 - protein, 15 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (14)..(14): Norleucine
CAPEKPYCFAQPYXC

SEQ ID NO: 266 - protein, 14 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (14)..(14): Norleucine modified with cysteamine
CAPEKPYCFADPYX

SEQ ID NO: 267 - protein, 15 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (14)..(14): Norleucine
  MOD_RES (15)..(15): 1,2-diaminoethane modified residue
CAPEKPYCFADPYXC

SEQ ID NO: 268 - protein, 17 aa, Artificial Sequence
  Description of Artificial Sequence: Synthetic peptide
  MOD_RES (1)..(1): Palmitoyl modified residue
  MOD_RES (2)..(2): Gamma-glutamic acid
  MOD_RES (4)..(4): t-butyl-alanine
  MOD_RES (7)..(7): 4-pentynoic acid modified D-amino acid
  MOD_RES (16)..(16): Norleucine
XXCAPEKPYCFADPYXC

SEQ ID NO: 269 - protein, 15 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (12)..(12): N-methyl-alanine
  MOD_RES (14)..(14): Norleucine
CAPEKPYCFADAYXC

SEQ ID NO: 270 - protein, 15 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (12)..(12): N-methyl-D-alanine
  MOD_RES (14)..(14): Norleucine
CAPEKPYCFADAYXC

SEQ ID NO: 271 - protein, 16 aa, Artificial Sequence
  Description of Artificial Sequence: Synthetic peptide
  N-term Ac
  MOD_RES (2)..(2): t-butyl-alanine
  MOD_RES (4)..(4): 4-pentynoic acid modified residue
  MOD_RES (5)..(5): D-amino acid
  MOD_RES (14)..(14): Norleucine
CAPKAPYCFADPYXCK

SEQ ID NO: 272 - protein, 16 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (14)..(14): Norleucine
  MOD_RES (16)..(16): D-amino acid
CAPEKPYCFADPYXCA

SEQ ID NO: 273 - protein, 16 aa, Artificial Sequence
  Description of Artificial Sequence: Synthetic peptide
  N-term Ac
  MOD_RES (2)..(2): t-butyl-alanine
  MOD_RES (5)..(5): 4-pentynoic acid modified D-amino acid
  MOD_RES (14)..(14): Norleucine
CAPEKPYCFADPYXCK

SEQ ID NO: 274 - protein, 16 aa, Artificial Sequence
  Description of Artificial Sequence: Synthetic peptide
  N-term Ac
  MOD_RES (2)..(2): t-butyl-alanine
  MOD_RES (5)..(5): Methylated D-amino acid modified with 4-pentynoic acid
  MOD_RES (14)..(14): Norleucine
  MOD_RES (16)..(16): D-amino acid
CAPEKPYCFADPYXCA

SEQ ID NO: 275 - protein, 15 aa, Artificial Sequence
  Description of Artificial Sequence: Synthetic peptide
  N-term Ac
  MOD_RES (2)..(2): t-butyl-alanine
  MOD_RES (5)..(5): Methylated D-amino acid modified with 4-pentynoic acid
  MOD_RES (14)..(14): Norleucine
CAPEKPYCFADPYXC

SEQ ID NO: 276 - protein, 5 aa, Artificial Sequence
  Description of Artificial Sequence: Synthetic peptide
  MOD_RES (1)..(1): PEG15-Sarcosine
  MOD_RES (2)..(2): Sarcosine
  MOD_RES (3)..(3): Sarcosine
  MOD_RES (4)..(4): Sarcosine
  MOD_RES (5)..(5): Sarcosine
XXXXX

SEQ ID NO: 277 - protein, 10 aa, Artificial Sequence
  Description of Artificial Sequence: Synthetic peptide
  MOD_RES (1)..(1): PEG10-Sarcosine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
XXXXXXXXXX

SEQ ID NO: 278 - protein, 15 aa, Artificial Sequence
  Description of Artificial Sequence: Synthetic peptide
  MOD_RES (1)..(1): PEG5-sarcosine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine
  MOD_RES (12)..(12): Sarcosine
  MOD_RES (13)..(13): Sarcosine
  MOD_RES (14)..(14): Sarcosine
  MOD_RES (15)..(15): Sarcosine
XXXXXXXXXXXXXXX

SEQ ID NO: 279 - protein, 5 aa, Artificial Sequence
  Description of Artificial Sequence: Synthetic peptide
  MOD_RES (1)..(1): PEG5-sarcosine
  MOD_RES (2)..(2): Sarcosine
  MOD_RES (3)..(3): Sarcosine
  MOD_RES (4)..(4): Sarcosine
  MOD_RES (5)..(5): Sarcosine
XXXXX

SEQ ID NO: 280 - protein, 21 aa, Artificial Sequence
  Description of Artificial Sequence: Synthetic peptide
  MOD_RES (1)..(1): Beta-alanine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine
  MOD_RES (12)..(12): Sarcosine
  MOD_RES (13)..(13): Sarcosine
  MOD_RES (14)..(14): Sarcosine
  MOD_RES (15)..(15): Sarcosine
  MOD_RES (16)..(16): Sarcosine
  MOD_RES (17)..(17): Sarcosine
  MOD_RES (18)..(18): Sarcosine
  MOD_RES (19)..(19): Sarcosine
  MOD_RES (20)..(20): Sarcosine
  MOD_RES (21)..(21): Sarcosine
AXXXXXXXXXXXXXXXXXXXX

SEQ ID NO: 281 - protein, 11 aa, Artificial Sequence
  Description of Artificial Sequence: Synthetic peptide
  MOD_RES (1)..(1): Beta-alanine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine
  MOD_RES (7)..(7): Sarcosine
  MOD_RES (8)..(8): Sarcosine
  MOD_RES (9)..(9): Sarcosine
  MOD_RES (10)..(10): Sarcosine
  MOD_RES (11)..(11): Sarcosine-PEG10
AXXXXXXXXXX

SEQ ID NO: 282 - protein, 6 aa, Artificial Sequence
  Description of Artificial Sequence: Synthetic peptide
  MOD_RES (1)..(1): Beta-alanine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine-PEG15
AXXXXX

SEQ ID NO: 283 - protein, 6 aa, Artificial Sequence
  Description of Artificial Sequence: Synthetic peptide
  MOD_RES (1)..(1): Beta-alanine
  MOD_RES (2)..(2): Sarcosine
  MOD_RES (3)..(3): Sarcosine
  MOD_RES (4)..(4): Sarcosine
  MOD_RES (5)..(5): Sarcosine
  MOD_RES (6)..(6): Sarcosine-PEG5
AXXXXX

(19) United States
(12) Patent Application Publication
(10) Pub. No.: US 2024/0325554 A1
(43) Pub. Date: Oct. 3, 2024

(54) METHODS FOR TREATING CANCER

(71) Applicant: Bicycle Tx Limited, Cambridge (GB)

(72) Inventors: Nicholas KEEN, Carlisle, MA (US); Gemma Elizabeth MUDD, Cambridge (GB); Johanna LAHDENRANTA, Cambridge (GB); Kristen HUROV, Cambridge (GB); Sailaja BATTULA, Cambridge (GB); Philip E. BRANDISH, Needham, MA (US); Punit UPADHYAYA, Cambridge (GB); Kevin MCDONNELL, Lexington, MA (US)

(21) Appl. No.: 18/271,593

(22) PCT Filed: Jan. 10, 2022
(86) PCT No.: PCT/GB2022/050055

§ 371 Date: Jul. 10, 2023

Related U.S. Application Data
(60) Provisional application No. 63/138,019, filed on Jan. 15, 2021.
(60) Provisional application No. 63/135,865, filed on Jan. 11, 2021.
(60) Provisional application No. 63/135,858, filed on Jan. 11, 2021.

(51) Int. Cl. A61K 47/66; A61K 39/00; A61K 39/395; A61P 35/00

(52) U.S. Cl. CPC A61K 47/66; A61K 39/3955; A61P 35/00; A61K 2039/505; A61K 2039/507

(57) ABSTRACT

The present invention relates to a method of treating a cancer in a patient.

### CROSS REFERENCE TO RELATED APPLICATIONS

This application is the national stage of International Patent Application No. PCT/GB2022/050055, filed Jan. 11, 2022, which claims the benefit of U.S. Provisional Application No. 63/138,019, filed Jan. 15, 2021, U.S. Provisional Application No. 63/135,865, filed Jan. 11, 2021, and U.S. Provisional Application No. 63/135,858, filed Jan. 11, 2021, the entire contents of each of which are incorporated herein by reference.

### SEQUENCE LISTING

The instant application contains a Sequence Listing which has been submitted electronically in ASCII format and is hereby incorporated by reference in its entirety. Said ASCII copy, created on Oct. 13, 2023, is named 201440_SL.txt and is 124,102 bytes in size.

### TECHNICAL FIELD OF THE INVENTION

The present invention relates to use of a heterotandem bicyclic peptide complex comprising one or more CD137 binding peptide ligand, or a pharmaceutically acceptable salt thereof, in combination with an immuno-oncology agent for treating cancer. The present invention also provides pharmaceutically acceptable compositions comprising a heterotandem bicyclic peptide complex comprising one or more CD137 binding peptide ligand, or a pharmaceutically acceptable salt thereof.

### BACKGROUND OF THE INVENTION

Cyclic peptides are able to bind with high affinity and target specificity to protein targets and hence are an attractive molecule class for the development of therapeutics. In fact, several cyclic peptides are already successfully used in the clinic, as for example the antibacterial peptide vancomycin, the immunosuppressant drug cyclosporine or the anti-cancer drug octreotide (Driggers et al. (2008), Nat Rev Drug Discov 7 (7), 608-24). Good binding properties result from a relatively large interaction surface formed between the peptide and the target as well as the reduced conformational flexibility of the cyclic structures. Typically, macrocycles bind to surfaces of several hundred square angstrom, as for example the cyclic peptide CXCR4 antagonist CVX15 (400 Å²; Wu et al. (2007), Science 330, 1066-71), a cyclic peptide with the Arg-Gly-Asp motif binding to integrin αVb3 (355 Å²) (Xiong et al. (2002), Science 296 (5565), 151-5) or the cyclic peptide inhibitor upain-1 binding to urokinase-type plasminogen activator (603 Å²; Zhao et al. (2007), J Struct Biol 160 (1), 1-10).

Due to their cyclic configuration, peptide macrocycles are less flexible than linear peptides, leading to a smaller loss of entropy upon binding to targets and resulting in a higher binding affinity. The reduced flexibility also leads to locking target-specific conformations, increasing binding specificity compared to linear peptides. This effect has been exemplified by a potent and selective inhibitor of matrix metalloproteinase 8 (MMP-8) which lost its selectivity over other MMPs when its ring was opened (Cherney et al. (1998), J Med Chem 41 (11), 1749-51). The favorable binding properties achieved through macrocyclization are even more pronounced in multicyclic peptides having more than one peptide ring as for example in vancomycin, nisin and actinomycin.

Different research teams have previously tethered polypeptides with cysteine residues to a synthetic molecular structure (Kemp and McNamara (1985), J. Org. Chem; Timmerman et al. (2005), ChemBioChem). Meloen and co-workers had used tris(bromomethyl)benzene and related molecules for rapid and quantitative cyclisation of multiple peptide loops onto synthetic scaffolds for structural mimicry of protein surfaces (Timmerman et al. (2005), ChemBioChem). Methods for the generation of candidate drug compounds wherein said compounds are generated by linking cysteine containing polypeptides to a molecular scaffold as for example tris(bromomethyl)benzene are disclosed in WO 2004/077062 and WO 2006/078161.

Phage display-based combinatorial approaches have been developed to generate and screen large libraries of bicyclic peptides to targets of interest (Heinis et al. (2009), Nat Chem Biol 5 (7), 502-7 and WO 2009/098450). Briefly, combinatorial libraries of linear peptides containing three cysteine residues and two regions of six random amino acids (Cys-(Xaa)₆-Cys-(Xaa)₆-Cys) were displayed on phage and cyclised by covalently linking the cysteine side chains to a small molecule (tris-(bromomethyl)benzene).

### SUMMARY OF THE INVENTION

It has now been found that a heterotandem bicyclic peptide complex comprising one or more CD137 binding peptide ligand, or a pharmaceutically acceptable salt thereof, leads to a significant increase of the tumor infiltrating immune cells and immune response. See, for example, the transcriptional analysis in Example 1 shows a significant increase in immune cell scores and mRNA for several T cell chemotactic chemokines/cytokines upon a treatment of each of BCY12491 and BT7480. Accordingly, in one aspect, the present invention provides a method for increasing immune response in a cancer patient, comprising administering to the patient a therapeutically effective amount of a heterotandem bicyclic peptide complex comprising one or more CD137 binding peptide ligand, or a pharmaceutically acceptable salt thereof.

It has also been found that a combination of a heterotandem bicyclic peptide complex comprising one or more CD137 binding peptide ligand, or a pharmaceutically acceptable salt thereof, and an immuno-oncology agent significantly improves anti-tumor activity compared to each of the single agent treatment. See, for example, a combination therapy of BCY12491 and a PD-1 antagonist Pembrolizumab in Example 2 leads to more significant anti-tumor activity compared to the treatment with each single agent. Accordingly, in one aspect, the present invention provides a method for treating a cancer in a patient, comprising administering to the patient a heterotandem bicyclic peptide complex comprising one or more CD137 binding peptide ligand, or a pharmaceutically acceptable salt thereof, and an immuno-oncology agent.

### BRIEF DESCRIPTION OF THE DRAWING

FIG. 1 depicts that BCY12491 modulates the tumor immune microenvironment and drives T cell infiltration. (A) MC38 tumor bearing mice were treated with vehicle, 15 mg/kg EphA2/CD137 heterotandem bicyclic peptide complex (BCY12491), an enantiomeric non-binding control heterotandem bicyclic peptide complex (BCY13626) q3d i.v. or 2 mg/kg αCD137 q3d i.p.. Individual tumor volumes (normalized to tumor volume on the day of treatment initiation) are shown grouped by treatment. (B) Nanostring analysis of tumors show the effect of BCY12491 and αCD137 on the T cell (probe set: Cd3d, Cd3e, Cd3g, Cd6, Sh2d1a and Trat1), cytotoxic cell (probe set: Ctsw, Gzma, Gzmb, Klrb1, Klrd1, Kirk1, Nkg7 and Prf1) and macrophage (probe set: Cd163, Cd68, Cd84 and Ms4a4a) content. (C) Nanostring analysis of tumors show the effect of BCY12491 and αCD137 on the checkpoint inhibitor Pdcd1 (protein PD-1), Cd274 (protein PD-L1) and Ctla4 (protein CTLA-4) transcription. (D) Representative images of tissue sections from tumors treated with vehicle, 15 mg/kg BCY12491, BCY13626 or 2 mg/kg αCD137 Q3D and stained for mouse CD8 are shown. (B and C) *<0.05, ***p<0.001, one-way ANOVA with Dunnett's post test.

FIG. 2 depicts the effect of BT7480 on a selected cytokines/chemokines. (A) Normalized linear count data is shown for 5 different cytokine/chemokine mRNAs in MC38 #13 tumor tissue after BT7480 treatment on the graph on the left hand side. (B) An overlay of the cytotoxic cell scores and Ccl1, Ccl-17 and Ccl24 normalized RNA counts demonstrate the early increase of those cytokine/chemokine transcripts followed by the increase in cytotoxic cell score.

FIG. 3 depicts that BT7480 modulates the tumor immune microenvironment and drive CD8+ T cell infiltration. MC38 #13 tumor bearing mice were treated with vehicle, 5 mg/kg (0 h, 24 h) of BT7480 or non-binding heterotandem bicyclic peptide complex control BCY12797 (NB-BCY) i.v. or 2 mg/kg αCD137 i.p.. Nanostring analysis of tumors show the effect of BT7480 and αCD137 on the (A) macrophage (probe set: Cd163, Cd68, Cd84 and Ms4a4a) and (B) cytotoxic cell (probe set: Ctsw, Gzma, Gzmb, Klrb1, Klrd1, Kirk1, Nkg7 and Prf1) scores in the tumor tissue over time. (C) Overlay of the cytotoxic cell scores and macrophage cell scores demonstrate the early increase of macrophage cell score followed by the increase in cytotoxic cell score. (A and B) *<0.05, **p<0.01, one-way ANOVA with Dunnett's post test.

FIG. 4 depicts that BT7480 leads to increase in several immune checkpoint mRNAs. MC38 #13 tumor bearing mice were treated with vehicle, 5 mg/kg (0 h, 24 h) of BT7480 or non-binding heterotandem bicyclic peptide complex control BCY12797 (NB-BCY) i.v. or 2 mg/kg αCD137 i.p.. Nanostring analysis of tumors show the effect of BT7480 and αCD137 on the levels of several immune checkpoint mRNAs. *<0.05, **p<0.01, ***p<0.001 one-way ANOVA with Dunnett's post test.

FIG. 5 depicts that BCY12491+Pembrolizumab combination from Day 0 (after treatment initiation) leads to 100% complete response rate by Day 22. MC38 tumor bearing mice were treated with vehicle, 5 mg/kg BCY12491 QW (0, 24 h), 3 mg/kg Pembrolizumab QW or their combination. The top graph shows the average tumor volumes from treatment initiation to Day 28. Both monotherapies and combination treatment significantly affected the tumor growth (***p<0.0001, mixed effects analysis with Dunnett's post test on D18 comparing to vehicle). Furthermore, the combination treatment was more efficacious than either one of the monotherapies (***p<0.0001, mixed effects analysis with Dunnett's post test on D20 comparing combination to monotherapies) leading to complete responses in all treated animals by day 22. Right hand side graphs show the growth curves of individual tumors from the treatment cohorts.

FIG. 6 depicts that BCY12491+Pembrolizumab combinations lead to significant anti-tumor activity with different dose sequencing. MC38 tumor bearing mice were treated with vehicle, 5 mg/kg BCY12491 QW (0, 24 h), 3 mg/kg Pembrolizumab QW or their combination with three different dosing schedules: both BCY12491 and Pembrolizumab treatment initiating on Day 0, BCY12491 treatment initiating on day 0 followed by Pembrolizumab treatment initiation on day 5, or Pembrolizumab treatment initiation on day 0 followed by BCY12491 treatment initiation on day 5. The top graph shows the average tumor volumes from treatment initiation to Day 28. All combination treatments show significant anti-tumor activity, with 10/10 (BCY12491+Pembrolizumab from DO), 9/10 (BCY12491 from DO+Pembrolizumab from D5) and 8/10 (Pembrolizumab from DO and BCY12491 from D5) complete responses by day 42. ***p<0.0001, mixed effects analysis with Dunnett's post test on D18 comparing to vehicle. Right hand side graphs show the growth curves of individual tumors from the treatment cohorts.

FIG. 7 depicts that addition of BCY11864 to anti-PD-1 monotherapy significantly [p=0.004, Log-rank (Mantel-Cox) test comparing anti-PD-1 and anti-PD-1+BCY11864 combination arms] increases the survival (defined as reaching humane endpoint, tumor volumes >2000 mm3) of CT26 #7 (CT26 engineered to overexpress Nectin-4) bearing mice.

FIG. 8 depicts that addition of BT7480 to anti-PD-1 monotherapy increases the rate of complete responses (CRs) in MC38 #13 (MC38 engineered to overexpress Nectin-4) bearing mice.

FIG. 9 depicts that addition of BT7480 to anti-CTLA-4 monotherapy significantly [p=0.0499, Log-rank (Mantel-Cox) test comparing anti-Ctla-4 and anti-Ctla-4+BT7480 combination arms] increases the survival (defined as reaching humane endpoint, tumor volumes >2000 mm³) of MC38 #13 (MC38 engineered to overexpress Nectin-4) bearing mice and increases the rate of complete responses.

FIG. 10 depicts that BT7455 leads to increase in several immune checkpoint mRNAs. MC38 tumor bearing mice were treated intravenously with vehicle, 8 mg/kg (0 h, 24 h) of BT7455 or intraperitoneally with 2 mg/kg anti-CD137 antibody or 10 mg/kg anti-PD-1 antibody. Nanostring analysis of tumors show the effect of the treatments on the levels of several immune checkpoint mRNAs. Normalized Log 2 count for mRNAs in MC38 tumor tissue at 24 hour, 48 hour and 144 hour timepoints are shown. *<0.05, **p<0.01, ***p<0.001 one-way ANOVA with Dunnett's post test comparing treatments to vehicle at the same timepoint.

FIG. 11 depicts that effect of BT7455 (8 mg/kg), anti-PD-1 and anti-CD137 (urelumab analogue) treatment on 5 selected cytokines/chemokines across 24 hour, 48 hour and 24 hour timepoints. Normalized Log 2 count for mRNAs in MC38 tumor tissue at 24 hour, 48 hour and 144 hour timepoints are shown. *p<0.05, **p<0.01, ****p<0.0001, 0.01 One-way ANOVA with Dunnett's post test.

FIG. 12 depicts that the effect of BT7455 (8 mg/kg), anti-PD-1 and anti-CD137 (urelumab analogue) treatment cytotoxic cells. The effects of treatments on cytotoxic cells at 24 hour, 48 hour and 144 hour timepoints are shown as Cytotoxic cell type score as normalized Log 2 (mean with standard deviation) scores in MC38 tumor tissue. (*p<0.05, One-way ANOVA with Dunnett's post test comparing the treatment to vehicle).

FIG. 13 depicts that transcriptional analysis revealed significant modulation (*p<0.05, **p<0.01 One-way ANOVA with Dunnett's post test) of several gene sets by BT7455 at an early timepoint (48 h) after treatment initiation whereas the effects of Anti-PD-1 and the urelumab analogue (anti-CD137) were not significant. The effects of the treatments on gene sets are shown as signature scores (mean with standard deviation) in MC38 tumor tissue.

### DETAILED DESCRIPTION OF CERTAIN EMBODIMENTS

### 1. Description of Certain Embodiments of the Invention

It has been found that a heterotandem bicyclic peptide complex comprising one or more CD137 binding peptide ligand, or a pharmaceutically acceptable salt thereof, leads to a significant increase of the tumor infiltrating immune cells and immune response, and that a combination of a heterotandem bicyclic peptide complex comprising one or more CD137 binding peptide ligand, or a pharmaceutically acceptable salt thereof, and an immuno-oncology agent significantly improves anti-tumor activity compared to each of the single agent treatment. See, for example, the data for a treatment with each of BCY12491 and BT7480 in Example 1, and the data in Example 2 for a treatment with BCY12491 alone, a PD-1 antagonist Pembrolizumab alone, and a combination of BCY12491 and Pembrolizumab. Accordingly, in one aspect, provided herein is a method or use of a heterotandem bicyclic peptide complex comprising one or more CD137 binding peptide ligand, or a pharmaceutically acceptable salt thereof, for increasing immune response in a cancer patient. In another aspect, provided herein is a method or use of a combination of a heterotandem bicyclic peptide complex comprising one or more CD137 binding peptide ligand, or a pharmaceutically acceptable salt thereof, and an immuno-oncology agent for treating a cancer in a patient.

In some embodiments, the present invention provides a method for increasing immune response in a cancer patient, comprising administering to the patient a therapeutically effective amount of a heterotandem bicyclic peptide complex comprising one or more CD137 binding peptide ligand, or a pharmaceutically acceptable salt thereof. In some embodiments, the present invention provides a use of a heterotandem bicyclic peptide complex comprising one or more CD137 binding peptide ligand, or a pharmaceutically acceptable salt thereof, in the manufacture of a medicament for increasing immune response in a cancer patient.

In some embodiments, the present invention provides a method for treating a cancer in a patient, comprising administering to the patient a therapeutically effective amount of a heterotandem bicyclic peptide complex comprising one or more CD137 binding peptide ligand, or a pharmaceutically acceptable salt thereof, and an immuno-oncology agent. In some embodiments, the present invention provides a use of a heterotandem bicyclic peptide complex comprising one or more CD137 binding peptide ligand, or a pharmaceutically acceptable salt thereof, in the manufacture of a medicament for treating a cancer in a patient, wherein the medicament is used in combination with an immuno-oncology agent.

In some embodiments, a cancer is selected from those as described herein. In some embodiments, a cancer is a solid tumor. In some embodiments, a cancer is associated with MT1-MMP. In some embodiments, the cancer is associated with Nectin-4. In some embodiments, the cancer is associated with EphA2. In some embodiments, the cancer is associated with PD-L1. In some embodiments, the cancer is associated with PSMA.

In some embodiments, a heterotandem bicyclic peptide complex comprising one or more CD137 binding peptide ligand is selected from the heterotandem bicyclic peptide complexes comprising one CD137 binding peptide ligand, as described herein. In some embodiments, a heterotandem bicyclic peptide complex comprising one or more CD137 binding peptide ligand is selected from the heterotandem bicyclic peptide complexes comprising two or more CD137 binding peptide ligands, as described herein.

In some embodiments, a heterotandem bicyclic peptide complex is BCY11863 (also referred to as BT7480), or a pharmaceutically acceptable salt thereof. In some embodiments, a heterotandem bicyclic peptide complex is BCY13272 (also referred to as BT7455), or a pharmaceutically acceptable salt thereof. In some embodiments, a heterotandem bicyclic peptide complex is BCY12491, or a pharmaceutically acceptable salt thereof. In some embodiments, a heterotandem bicyclic peptide complex is BCY11864, or a pharmaceutically acceptable salt thereof.

In some embodiments, an immuno-oncology agent is selected from those as described herein. In some embodiments, an immuno-oncology agent is a check point inhibitor. In some embodiments, an immuno-oncology agent is a PD-1 antagonist. In some embodiments, an immuno-oncology agent is pembrolizumab. In some embodiments, an immuno-oncology agent is nivolumab.

In some embodiments, the present invention provides a method for increasing immune response in a cancer patient, comprising administering to the patient a therapeutically effective amount of BT7480, or a pharmaceutically acceptable salt thereof. In some embodiments, the present invention provides a use of BT7480, or a pharmaceutically acceptable salt thereof, in the manufacture of a medicament for increasing immune response in a cancer patient. In some embodiments, the present invention provides a method for treating a cancer in a patient, comprising administering to the patient a therapeutically effective amount of BT7480, or a pharmaceutically acceptable salt thereof, and an immuno-oncology agent. In some embodiments, the present invention provides a use of BT7480, or a pharmaceutically acceptable salt thereof, in the manufacture of a medicament for treating a cancer in a patient, wherein the medicament is used in combination with an immuno-oncology agent.

In some embodiments, the present invention provides a method for increasing immune response in a cancer patient, comprising administering to the patient a therapeutically effective amount of BT7455, or a pharmaceutically acceptable salt thereof. In some embodiments, the present invention provides a use of BT7455, or a pharmaceutically acceptable salt thereof, in the manufacture of a medicament for increasing immune response in a cancer patient. In some embodiments, the present invention provides a method for treating a cancer in a patient, comprising administering to the patient a therapeutically effective amount of BT7455, or a pharmaceutically acceptable salt thereof, and an immuno-oncology agent. In some embodiments, the present invention provides a use of BT7455, or a pharmaceutically acceptable salt thereof, in the manufacture of a medicament for treating a cancer in a patient, wherein the medicament is used in combination with an immuno-oncology agent.

In some embodiments, a heterotandem bicyclic peptide complex is administered at a dose of about 0.001-100 mg/kg. In some embodiments, a heterotandem bicyclic peptide complex is selected from those described herein, for example, BT7480 or BT7455, or a pharmaceutically acceptable salt thereof. In some embodiments, a heterotandem bicyclic peptide complex is administered at a dose of about 0.001-0.01 mg/kg, about 0.01-0.1 mg/kg, about 0.1-1 mg/kg, about 1-10 mg/kg, about 10-25 mg/kg, about 25-50 mg/kg, or about 50-100 mg/kg. In some embodiments, a heterotandem bicyclic peptide complex is administered at a dose of about 0.1-75 mg/kg, about 1-50 mg/kg, about 5-25 mg/kg, or about 7.5-20 mg/kg. In some embodiments, a heterotandem bicyclic peptide complex is administered at a dose of about 0.001 mg/kg, about 0.005 mg/kg, about 0.01 mg/kg, about 0.05 mg/kg, about 0.1 mg/kg, about 0.25 mg/kg, about 0.5 mg/kg, about 1 mg/kg, about 3 mg/kg, about 5 mg/kg, about 7.5 mg/kg, about 10 mg/kg, about 12.5 mg/kg, about 15 mg/kg, about 20 mg/kg, about 25 mg/kg, about 30 mg/kg, about 40 mg/kg, or about 50 mg/kg.

In some embodiments, a heterotandem bicyclic peptide complex is administered at a frequency of 1, 2, 3, or 4 times a week. In some embodiments, a heterotandem bicyclic peptide complex is selected from those described herein, for example, BT7480 or BT7455, or a pharmaceutically acceptable salt thereof. In some embodiments, a heterotandem bicyclic peptide complex is administered once daily. In some embodiments, a heterotandem bicyclic peptide complex is administered once every 2 days. In some embodiments, a heterotandem bicyclic peptide complex is administered once every 3 days. In some embodiments, a heterotandem bicyclic peptide complex is administered once every 4 days. In some embodiments, a heterotandem bicyclic peptide complex is administered once every 5 days. In some embodiments, a heterotandem bicyclic peptide complex is administered at a frequency of once a week. In some embodiments, a heterotandem bicyclic peptide complex is administered at a frequency of once every 1.5 weeks. In some embodiments, a heterotandem bicyclic peptide complex is administered at a frequency of once every 2 weeks. In some embodiments, a heterotandem bicyclic peptide complex is administered at a frequency of once every 2.5 weeks. In some embodiments, a heterotandem bicyclic peptide complex is administered at a frequency of once every 3 weeks. In some embodiments, a heterotandem bicyclic peptide complex is administered at a frequency of once every 4 weeks. In some embodiments, a heterotandem bicyclic peptide complex is administered at a frequency of once a month.

In some embodiments, a heterotandem bicyclic peptide complex is administered for a treatment period of about 1-4 weeks. In some embodiments, a heterotandem bicyclic peptide complex is selected from those described herein, for example, BT7480 or BT7455, or a pharmaceutically acceptable salt thereof. In some embodiments, a heterotandem bicyclic peptide complex is administered for a treatment period of about 5-8 weeks. In some embodiments, a heterotandem bicyclic peptide complex is administered for a treatment period of about 9-12 weeks. In some embodiments, a heterotandem bicyclic peptide complex is administered for a treatment period of about 13-20 weeks. In some embodiments, a heterotandem bicyclic peptide complex is administered for a treatment period of about 21-28 weeks. In some embodiments, a heterotandem bicyclic peptide complex is administered for a treatment period of about 4, 8, 12, 16, 20, 24, or 28 weeks. In some embodiments, a heterotandem bicyclic peptide complex is administered for a treatment period of about 30 weeks, or longer.

In some embodiments, a heterotandem bicyclic peptide complex is administered to a patient by an intravenous bolus injection. In some embodiments, a heterotandem bicyclic peptide complex is selected from those described herein, for example, BT7480 or BT7455, or a pharmaceutically acceptable salt thereof. In some embodiments, a heterotandem bicyclic peptide complex is administered to a patient by an intravenous infusion. In some embodiments, an intravenous infusion of a heterotandem bicyclic peptide complex is an about 5-10 minute infusion. In some embodiments, an intravenous infusion of a heterotandem bicyclic peptide complex is an about 10-20 minute infusion. In some embodiments, an intravenous infusion of a heterotandem bicyclic peptide complex is an about 20-40 minute infusion. In some embodiments, an intravenous infusion of a heterotandem bicyclic peptide complex is an about 45, or 50, or 55 minute infusion. In some embodiments, an intravenous infusion of a heterotandem bicyclic peptide complex is an about 1 hour infusion. In some embodiments, an intravenous infusion of a heterotandem bicyclic peptide complex is an about 1-1.5 hr infusion. In some embodiments, an intravenous infusion of a heterotandem bicyclic peptide complex is an about 1.5-2 hr infusion. In some embodiments, an intravenous infusion of a heterotandem bicyclic peptide complex is an about 2-3 hr infusion. In some embodiments, an intravenous infusion of a heterotandem bicyclic peptide complex is a more than 3 hr infusion.

An immuno-oncology agent is administered at the dosage regimen according to FDA recommendation or approval. In some embodiments, an immuno-oncology agent is administered at a dose of about 1-20 mg/kg. In some embodiments, an immuno-oncology agent is administered at a dose of about 1-5 mg/kg, about 6-10 mg/kg, about 11-15 mg/kg, or about 16-20 mg/kg. In some embodiments, an immuno-oncology agent is administered at a dose of about 1-10 mg/kg, about 5-15 mg/kg, or about 10-20 mg/kg. In some embodiments, an immuno-oncology agent is administered at a dose of about 2, 3, 4, 5, 6, 7, 8, 9, or 10 mg/kg. In some embodiments, an immuno-oncology agent is administered at a dose of about 11, 12, 13, 14, 15, 16, 17, 18, 19, or 20 mg/kg. In some embodiments, an immuno-oncology agent is administered at a frequency of 1, 2, 3, or 4 times a week. In some embodiments, an immuno-oncology agent is administered once daily. In some embodiments, an immuno-oncology agent is administered once every 2 days. In some embodiments, an immuno-oncology agent is administered once every 3 days. In some embodiments, an immuno-oncology agent is administered once every 4 days. In some embodiments, an immuno-oncology agent is administered once every 5 days. In some embodiments, an immuno-oncology agent is administered at a frequency of once a week. In some embodiments, an immuno-oncology agent is administered at a frequency of once every 1.5 weeks. In some embodiments, an immuno-oncology agent is administered at a frequency of once every 2 weeks. In some embodiments, an immuno-oncology agent is administered at a frequency of once every 2.5 weeks. In some embodiments, an immuno-oncology agent is administered at a frequency of once every 3 weeks. In some embodiments, an immuno-oncology agent is administered at a frequency of once every 4 weeks. In some embodiments, an immuno-oncology agent is administered at a frequency of once a month. In some embodiments, an immuno-oncology agent is administered for a treatment period of about 1-4 weeks. In some embodiments, an immuno-oncology agent is administered for a treatment period of about 9-12 weeks, about 13-20 weeks, about 21-28 weeks, or about 29-36 weeks. In some embodiments, an immuno-oncology agent is administered for a treatment period of about 36 weeks, or longer. In some embodiments, an immuno-oncology agent is administered to a patient by an intravenous injection. In some embodiments, an immuno-oncology agent is administered to a patient by an intravenous infusion. In some embodiments, an intravenous infusion of an immuno-oncology agent is an about 5-10 minute infusion. In some embodiments, an intravenous infusion of an immuno-oncology agent is an about 10-20 minute or about 20-40 minute infusion. In some embodiments, an intravenous infusion of an immuno-oncology agent is an about 30, 40, 45, 50, 55, or 60 minute infusion. In some embodiments, an intravenous infusion of an immuno-oncology agent is an about 1-1.5 hr, about 1.5-2 hr, or about 2-3 hr infusion.

In some embodiments, a medicament comprising a heterotandem bicyclic peptide complex, or a pharmaceutically acceptable salt thereof, is selected from the heterotandem bicyclic peptide complex formulations as shown in the instant examples. In some embodiments, a heterotandem bicyclic peptide complex is selected from those described herein, for example, BT7480 or BT7455, or a pharmaceutically acceptable salt thereof. In some embodiments, a medicament comprising a heterotandem bicyclic peptide complex, or a pharmaceutically acceptable salt thereof, further comprises histidine. In some embodiments, a medicament comprising a heterotandem bicyclic peptide complex, or a pharmaceutically acceptable salt thereof, and histidine is at about pH 7. In some embodiments, a medicament comprising a heterotandem bicyclic peptide complex, or a pharmaceutically acceptable salt thereof, further comprises sucrose. In some embodiments, a medicament comprising a heterotandem bicyclic peptide complex, or a pharmaceutically acceptable salt thereof, further comprises about 10% w/v sucrose. In some embodiments, a medicament comprising a heterotandem bicyclic peptide complex, or a pharmaceutically acceptable salt thereof, further comprises water. In some embodiments, the present invention provides a medicament comprising a heterotandem bicyclic peptide complex, or a pharmaceutically acceptable salt thereof, histidine, sucrose, and water, wherein the medicament is at about pH 7.

### Exemplary Heterotandem Bicyclic Peptide Complexes

In some embodiments, a heterotandem bicyclic peptide complex, or a pharmaceutically acceptable salt thereof, comprises:
(a) a first peptide ligand which binds to a component present on a cancer cell; conjugated via a linker to
(b) one or more CD137 binding peptide ligands;
   wherein each of said peptide ligands comprise a polypeptide comprising at least three reactive groups, separated by at least two loop sequences, and a molecular scaffold which forms covalent bonds with the reactive groups of the polypeptide such that at least two polypeptide loops are formed on the molecular scaffold.

In some embodiments, a heterotandem bicyclic peptide complex, or a pharmaceutically acceptable salt thereof, comprises:
(a) a first peptide ligand which binds to a component present on a cancer cell; conjugated via a linker to
(b) one or more CD137 binding peptide ligands;
   wherein each of said peptide ligands comprise a polypeptide comprising at least three cysteine residues, separated by at least two loop sequences, and a molecular scaffold which forms covalent bonds with the cysteine residues of the polypeptide such that at least two polypeptide loops are formed on the molecular scaffold.

In some embodiments, a heterotandem bicyclic peptide complex, or a pharmaceutically acceptable salt thereof, comprises:
(a) a first peptide ligand which binds to a component present on a cancer cell; conjugated via a linker to
(b) one CD137 binding peptide ligand;
   wherein each of said peptide ligands comprise a polypeptide comprising at least three reactive groups, separated by at least two loop sequences, and a molecular scaffold which forms covalent bonds with the reactive groups of the polypeptide such that at least two polypeptide loops are formed on the molecular scaffold.

In some embodiments, a heterotandem bicyclic peptide complex, or a pharmaceutically acceptable salt thereof, comprises:
(a) a first peptide ligand which binds to a component present on a cancer cell; conjugated via a linker to
(b) one CD137 binding peptide ligand;
   wherein each of said peptide ligands comprise a polypeptide comprising at least three cysteine residues, separated by at least two loop sequences, and a molecular scaffold which forms covalent bonds with the cysteine residues of the polypeptide such that at least two polypeptide loops are formed on the molecular scaffold.

In some embodiments, a heterotandem bicyclic peptide complex, or a pharmaceutically acceptable salt thereof, comprises:
(a) a first peptide ligand which binds to a component present on a cancer cell; conjugated via a linker to
(b) two or more CD137 binding peptide ligands;
   wherein each of said peptide ligands comprise a polypeptide comprising at least three reactive groups, separated by at least two loop sequences, and a molecular scaffold which forms covalent bonds with the reactive groups of the polypeptide such that at least two polypeptide loops are formed on the molecular scaffold.

In some embodiments, a heterotandem bicyclic peptide complex, or a pharmaceutically acceptable salt thereof, comprises:
(a) a first peptide ligand which binds to a component present on a cancer cell; conjugated via a linker to
(b) two or more CD137 binding peptide ligands;
   wherein each of said peptide ligands comprise a polypeptide comprising at least three cysteine residues, separated by at least two loop sequences, and a molecular scaffold which forms covalent bonds with the cysteine residues of the polypeptide such that at least two polypeptide loops are formed on the molecular scaffold.

In some embodiments, a heterotandem bicyclic peptide complex, or a pharmaceutically acceptable salt thereof, comprises:
(a) a first peptide ligand which binds to a component present on a cancer cell; conjugated via a linker to
(b) two CD137 binding peptide ligands;
   wherein each of said peptide ligands comprise a polypeptide comprising at least three reactive groups, separated by at least two loop sequences, and a molecular scaffold which forms covalent bonds with the reactive groups of the polypeptide such that at least two polypeptide loops are formed on the molecular scaffold.

In some embodiments, a heterotandem bicyclic peptide complex, or a pharmaceutically acceptable salt thereof, comprises:
(a) a first peptide ligand which binds to a component present on a cancer cell; conjugated via a linker to
(b) two CD137 binding peptide ligands;
   wherein each of said peptide ligands comprise a polypeptide comprising at least three cysteine residues, separated by at least two loop sequences, and a molecular scaffold which forms covalent bonds with the cysteine residues of the polypeptide such that at least two polypeptide loops are formed on the molecular scaffold.

In some embodiments, a heterotandem bicyclic peptide complex, or a pharmaceutically acceptable salt thereof, comprises:
(a) a first peptide ligand which binds to a component present on a cancer cell; conjugated via a linker to
(b) three CD137 binding peptide ligands;
   wherein each of said peptide ligands comprise a polypeptide comprising at least three reactive groups, separated by at least two loop sequences, and a molecular scaffold which forms covalent bonds with the reactive groups of the polypeptide such that at least two polypeptide loops are formed on the molecular scaffold.

In some embodiments, a heterotandem bicyclic peptide complex, or a pharmaceutically acceptable salt thereof, comprises:
(a) a first peptide ligand which binds to a component present on a cancer cell; conjugated via a linker to
(b) three CD137 binding peptide ligands;
   wherein each of said peptide ligands comprise a polypeptide comprising at least three cysteine residues, separated by at least two loop sequences, and a molecular scaffold which forms covalent bonds with the cysteine residues of the polypeptide such that at least two polypeptide loops are formed on the molecular scaffold.

### First Peptide Ligands

References herein to the term “cancer cell” includes any cell which is known to be involved in cancer. Cancer cells are created when the genes responsible for regulating cell division are damaged. Carcinogenesis is caused by mutation and epimutation of the genetic material of normal cells, which upsets the normal balance between proliferation and cell death. This results in uncontrolled cell division and the evolution of those cells by natural selection in the body. The uncontrolled and often rapid proliferation of cells can lead to benign or malignant tumors (cancer). Benign tumors do not spread to other parts of the body or invade other tissues. Malignant tumors can invade other organs, spread to distant locations (metastasis) and become life-threatening.

In some embodiments, the cancer cell is selected from an HT1080, A549, SC-OV-3, PC3, HT1376, NCI-H292, LnCap, MC38, MC38 #13, 4T1-D02, H322, HT29, T47D and RKO tumor cell.

In some embodiments, a component present on a cancer cell is Nectin-4.

Nectin-4 is a surface molecule that belongs to the nectin family of proteins, which comprises 4 members. Nectins are cell adhesion molecules that play a key role in various biological processes such as polarity, proliferation, differentiation and migration, for epithelial, endothelial, immune and neuronal cells, during development and adult life. They are involved in several pathological processes in humans. They are the main receptors for poliovirus, herpes simplex virus and measles virus. Mutations in the genes encoding Nectin-1 (PVRL1) or Nectin-4 (PVRL4) cause ectodermal dysplasia syndromes associated with other abnormalities. Nectin-4 is expressed during foetal development. In adult tissues its expression is more restricted than that of other members of the family. Nectin-4 is a tumor-associated antigen in 50%, 49% and 86% of breast, ovarian and lung carcinomas, respectively, mostly on tumors of bad prognosis. Its expression is not detected in the corresponding normal tissues. In breast tumors, Nectin-4 is expressed mainly in triple-negative and ERBB2+ carcinomas. In the serum of patients with these cancers, the detection of soluble forms of Nectin-4 is associated with a poor prognosis. Levels of serum Nectin-4 increase during metastatic progression and decrease after treatment. These results suggest that Nectin-4 could be a reliable target for the treatment of cancer. Accordingly, several anti-Nectin-4 antibodies have been described in the prior art. In particular, Enfortumab Vedotin (ASG-22ME) is an antibody-drug conjugate (ADC) targeting Nectin-4 and is currently clinically investigated for the treatment of patients suffering from solid tumors.

In some embodiments, the first peptide ligand comprises a Nectin-4 binding bicyclic peptide ligand.

In some embodiments, a Nectin-4 binding bicyclic peptide ligand is selected from those disclosed in WO 2019/243832, the contents of which are incorporated herein by reference in their entireties.

In some embodiments, a Nectin-4 binding bicyclic peptide ligand comprises an amino acid sequence selected from:

CᵢP[1Nal][dD]CᵢᵢM[HArg]DWSTP[HyP]WCᵢᵢᵢ
(SEQ ID NO: 1; herein referred to as BCY8116);
(SEQ ID NO: 2)
CᵢP[1Nal][dD]CᵢᵢM[HArg]D[dW]STP[HyP][dW]Cᵢᵢᵢ;
(SEQ ID NO: 3)
CᵢP[1Nal][dK](Sar₁₀-(B-Ala))CᵢᵢM[HArg]DWSTP[HyP]
WCᵢᵢᵢ;
CᵢPFGCᵢᵢM[HArg]DWSTP[HyP]WCᵢᵢᵢ
(SEQ ID NO: 4; herein referred to as BCY11414);
(SEQ ID NO: 14)
CᵢP[1Nal][dK]CᵢᵢM[HArg]DWSTP[HyP]WCᵢᵢᵢ;
[MerPro];P[1Nal][dK]CᵢᵢM[HArg]DWSTP[HyP]WCᵢᵢᵢ
(SEQ ID NO: 15; herein referred to as BCY12363);
(SEQ ID NO: 16)
CᵢP[1Nal][dK]CᵢᵢM[HArg]DWSTP[HyP]W[Cysam]ᵢᵢᵢ;
[MerPro];P[1Nal][dK]CᵢᵢM[HArg]DWSTP[HyP]W[Cysam]ᵢᵢᵢ
(SEQ ID NO: 17; herein referred to as BCY12365);
(SEQ ID NO: 18)
CᵢP[1Nal][dK]CᵢᵢM[HArg]HWSTP[HyP]WCᵢᵢᵢ;
(SEQ ID NO: 19)
CᵢP[1Nal][dK]CᵢᵢM[HArg]EWSTP[HyP]WCᵢᵢᵢ;
CᵢP[1Nal][dE]CᵢᵢM[HArg]DWSTP[HyP]WCᵢᵢᵢ
(SEQ ID NO: 20; herein referred to as BCY12368);
CᵢP[1Nal][dA]CᵢᵢM[HArg]DWSTP[HyP]WCᵢᵢᵢ
(SEQ ID NO: 21; herein referred to as BCY12369);
CᵢP[1Nal][dE]CᵢᵢL[HArg]DWSTP[HyP]WCᵢᵢᵢ
(SEQ ID NO: 22; herein referred to as BCY12370);
and
CᵢP[1Nal][dE]CᵢᵢM[HArg]EWSTP[HyP]WCᵢᵢᵢ
(SEQ ID NO: 23; herein referred to as BCY12384);

wherein [MerPro]ᵢ, Cᵢ, Cᵢᵢ, Cᵢᵢᵢ and [Cysam]ᵢᵢᵢ represent first (i), second (ii) and third (iii) reactive groups which are selected from cysteine, MerPro and Cysam, 1Nal represents 1-naphthylalanine, HArg represents homoarginine, HyP represents trans-4-hydroxy-L-proline, Sar₁₀ represents 10 sarcosine units, B-Ala represents beta-alanine, MerPro represents 3-mercaptopropionic acid and Cysam represents cysteamine, or a pharmaceutically acceptable salt thereof.

In some embodiments, a Nectin-4 binding bicyclic peptide ligand comprises an amino acid sequence selected from:

CᵢP[1Nal][dD]CᵢᵢM[HArg]DWSTP[HyP]WCᵢᵢᵢ
(SEQ ID NO: 1; herein referred to as BCY8116);
(SEQ ID NO: 3)
CᵢP[1Nal][dK](Sar₁₀-(B-Ala))CᵢᵢM[HArg]DWSTP[HyP]
WCᵢᵢᵢ;
and
CᵢPFGCᵢᵢM[HArg]DWSTP[HyP]WCᵢᵢᵢ
(SEQ ID NO: 4; herein referred to as BCY11414);

wherein Cᵢ, Cᵢᵢ and Cᵢᵢᵢ represent first, second and third cysteine residues, respectively, 1Nal represents 1-naphthylalanine, HArg represents homoarginine, HyP represents trans-4-hydroxy-L-proline, Sar₁₀ represents 10 sarcosine units, B-Ala represents beta-alanine, or a pharmaceutically acceptable salt thereof.

In some embodiments, a Nectin-4 binding bicyclic peptide ligand optionally comprises N-terminal modifications and comprises:

SEQ ID NO: 1 (herein referred to as BCY8116);
[PYA]-[B-Ala]-[Sar₁₀]-(SEQ ID NO: 1)
(SEQ ID NO: 165) (herein referred to as BCY8846);
(SEQ ID NO: 166)
[PYA]-(SEQ ID NO: 1)
(herein referred to as BCY11015);
(SEQ ID NO: 167)
[PYA]-[B-Ala]-(SEQ ID NO: 1)
(herein referred to as BCY11016);
(SEQ ID NO: 168)
[PYA]-[B-Ala]-[Sar₁₀]-(SEQ ID NO: 2)
(herein referred to as BCY11942);
(SEQ ID NO: 169)
Ac-(SEQ ID NO: 3)
(herein referred to as BCY8831);
SEQ ID NO: 4
herein referred to as BCY11414);
(SEQ ID NO: 170)
[PYA]-[B-Ala]-(SEQ ID NO: 14)
(herein referred to as BCY11143);
(SEQ ID NO: 171)
Palmitic-yGlu-yGlu-(SEQ ID NO: 14)
(herein referred to as BCY12371);
(SEQ ID NO: 172)
Ac-(SEQ ID NO: 14)
(herein referred to as BCY12024);
(SEQ ID NO: 173)
Ac-(SEQ ID NO: 16)
(herein referred to as BCY12364);
(SEQ ID NO: 174)
Ac-(SEQ ID NO: 18)
(herein referred to as BCY12366);
and
(SEQ ID NO: 175)
Ac-(SEQ ID NO: 19)
(herein referred to as BCY12367);

wherein PYA represents 4-pentynoic acid, B-Ala represents beta-alanine, Sar₁₀ represents 10 sarcosine units, or a pharmaceutically acceptable salt thereof.

In some embodiments, a Nectin-4 binding bicyclic peptide ligand optionally comprises N-terminal modifications and comprises:

SEQ ID NO: 1 (herein referred to as BCY8116);
(SEQ ID NO: 165)
[PYA]-[B-Ala]-[Sar₁₀]-(SEQ ID NO: 1)
(herein referred to as BCY8846);
(SEQ ID NO: 168)
[PYA]-[B-Ala]-[Sar₁₀]-(SEQ ID NO: 2)
(herein referred to as BCY11942);
(SEQ ID NO: 169)
Ac-(SEQ ID NO: 3)
(herein referred to as BCY8831);
and
SEQ ID NO: 4 (herein referred to as BCY11414);

wherein PYA represents 4-pentynoic acid, B-Ala represents beta-alanine, Sar₁₀ represents 10 sarcosine units, or a pharmaceutically acceptable salt thereof.

In some embodiments, a Nectin-4 binding bicyclic peptide ligand comprises SEQ ID NO: 1 (herein referred to as BCY8116).

In some embodiments, a Nectin-4 binding bicyclic peptide ligand comprises an amino acid sequence selected from:

CᵢP[1Nal][dD]CᵢᵢM[HArg]DWSTP[HyP]WCᵢᵢᵢ
(SEQ ID NO: 1; hereinafter referred to as
BCY8116);
CᵢP[1Nal][dD]CᵢᵢM[HArg]D[dW]STP[HyP][dW]Cᵢᵢᵢ
(SEQ ID NO: 2; hereinafter referred to as
BCY11415);
and
(SEQ ID NO: 3)
CᵢP[1Nal][dK](Sar₁₀-(B-Ala))CᵢᵢM[HArg]DWSTP[HyP]
WCᵢᵢᵢ;
CᵢPFGCᵢᵢP[HArg]DWSTP[HyP]WCᵢᵢᵢ
(SEQ ID NO: 4; hereinafter referred to as
BCY11414);

wherein Cᵢ, Cᵢᵢ and Cᵢᵢᵢ represent first, second and third cysteine residues, respectively, 1Nal represents 1-naphthylalanine, HArg represents homoarginine, HyP represents hydroxyproline, Sar₁₀ represents 10 sarcosine units, B-Ala represents beta-alanine, or a pharmaceutically acceptable salt thereof.

In a further embodiment, the Nectin-4 binding bicyclic peptide ligand optionally comprises N-terminal modifications and comprises: PGP-26 DNA SEQ ID NO: 1 (hereinafter referred to as BCY8116);

SEQ ID NO: 1
(hereinafter referred to as BCY8116);
(SEQ ID NO: 165)
[PYA]-[B-Ala]-[Sar₁₀]-(SEQ ID NO: 1)
(hereinafter referred to as BCY8846);
SEQ ID NO: 2 (hereinafter referred to as
BCY11415);
(SEQ ID NO: 168)
[PYA]-[B-Ala]-[Sar₁₀]-(SEQ ID NO: 2)
(hereinafter referred to as BCY11942);
(SEQ ID NO: 169)
Ac-(SEQ ID NO: 3)(hereinafter referred to as
BCY8831);
and
SEQ ID NO: 4
(hereinafter referred to as BCY11414);

wherein PYA represents 4-pentynoic acid, B-Ala represents beta-alanine, Sar₁₀ represents 10 sarcosine units, or a pharmaceutically acceptable salt thereof.

In some embodiments, a component present on a cancer cell is EphA2.

Eph receptor tyrosine kinases (Ephs) belong to a large group of receptor tyrosine kinases (RTKs), kinases that phosphorylate proteins on tyrosine residues. Ephs and their membrane bound ephrin ligands (ephrins) control cell positioning and tissue organization (Poliakov et al. (2004) Dev Cell 7, 465-80). Functional and biochemical Eph responses occur at higher ligand oligomerization states (Stein et al. (1998) Genes Dev 12, 667-678).

Among other patterning functions, various Ephs and ephrins have been shown to play a role in vascular development. Knockout of EphB4 and ephrin-B2 results in a lack of the ability to remodel capillary beds into blood vessels (Poliakov et al., supra) and embryonic lethality. Persistent expression of some Eph receptors and ephrins has also been observed in newly-formed, adult micro-vessels (Brantley-Sieders et al. (2004) Curr Pharm Des 10, 3431-42; Adams (2003) J Anat 202, 105-12).

The de-regulated re-emergence of some ephrins and their receptors in adults also has been observed to contribute to tumor invasion, metastasis and neo-angiogenesis (Nakamoto et al. (2002) Microsc Res Tech 59, 58-67; Brantley-Sieders et al., supra). Furthermore, some Eph family members have been found to be over-expressed on tumor cells from a variety of human tumors (Brantley-Sieders et al., supra); Marine (2002) Ann Hematol 81 Suppl 2, S66; Booth et al. (2002) Nat Med 8, 1360-1).

EPH receptor A2 (ephrin type-A receptor 2) is a protein that in humans is encoded by the EPHA2 gene.

EphA2 is upregulated in multiple cancers in man, often correlating with disease progression, metastasis and poor prognosis e.g.: breast (Zelinski et al (2001) Cancer Res. 61, 2301-2306; Zhuang et al (2010) Cancer Res. 70, 299-308; Brantley-Sieders et al (2011) PLoS One 6, e24426), lung (Brannan et al (2009) Cancer Prev Res (Phila) 2, 1039-1049; Kinch et al (2003) Clin Cancer Res. 9, 613-618; Guo et al (2013) J Thorac Oncol. 8, 301-308), gastric (Nakamura et al (2005) Cancer Sci. 96, 42-47; Yuan et al (2009) Dig Dis Sci 54, 2410-2417), pancreatic (Mudali et al (2006) Clin Exp Metastasis 23, 357-365), prostate (Walker-Daniels et al (1999) Prostate 41, 275-280), liver (Yang et al (2009) Hepatol Res. 39, 1169-1177) and glioblastoma (Wykosky et al (2005) Mol Cancer Res. 3, 541-551; Li et al (2010) Tumor Biol. 31, 477-488).

The full role of EphA2 in cancer progression is still not defined although there is evidence for interaction at numerous stages of cancer progression including tumor cell growth, survival, invasion and angiogenesis. Downregulation of EphA2 expression suppresses tumor cancer cell propagation (Binda et al (2012) Cancer Cell 22, 765-780), whilst EphA2 blockade inhibits VEGF induced cell migration (Hess et al (2001) Cancer Res. 61, 3250-3255), sprouting and angiogenesis (Cheng et al (2002) Mol Cancer Res. 1, 2-11; Lin et al (2007) Cancer 109, 332-40) and metastatic progression (Brantley-Sieders et al (2005) FASEB J. 19, 1884-1886).

An antibody drug conjugate to EphA2 has been shown to significantly diminish tumor growth in rat and mouse xenograft models (Jackson et al (2008) Cancer Research 68, 9367-9374) and a similar approach has been tried in man although treatment had to be discontinued for treatment related adverse events (Annunziata et al (2013) Invest New drugs 31, 77-84).

In some embodiments, the first peptide ligand comprises an EphA2 binding bicyclic peptide ligand.

In some embodiments, an EphA2 binding bicyclic peptide ligands is selected from those disclosed in WO 2019/122860, WO 2019/122861 and WO 2019/122863, the contents of each of which are incorporated herein by reference in their entireties.

In some embodiments, an EphA2 binding bicyclic peptide ligand comprises an amino acid sequence selected from:

(SEQ ID NO: 24)
Cᵢ[HyP]LVNPLCᵢᵢLHP[dD]W[HArg]Cᵢᵢᵢ;
(SEQ ID NO: 25)
CᵢLWDPTPCᵢᵢANLHL[HArg]Cᵢᵢᵢ;
(SEQ ID NO: 26)
Cᵢ[HyP]LVNPLCᵢᵢL[K(PYA)]P[dD]W[HArg]Cᵢᵢᵢ;
(SEQ ID NO: 27)
Cᵢ[HyP][K(PYA)]VNPLCᵢᵢLHP[dD]W[HArg]Cᵢᵢᵢ;
(SEQ ID NO: 28)
Cᵢ[HyP]LVNPLCᵢᵢ[K(PYA)]HP[dD]W[HArg]Cᵢᵢᵢ;
(SEQ ID NO: 29)
Cᵢ[HyP]LVNPLCᵢᵢLKP[dD]W[HArg]Cᵢᵢᵢ;
(SEQ ID NO: 30)
Cᵢ[HyP]KVNPLCᵢᵢLHP[dD]W[HArg]Cᵢᵢᵢ;
(SEQ ID NO: 31)
Cᵢ[HyP]LVNPLCᵢᵢKHP[dD]W[HArg]Cᵢᵢᵢ;
(SEQ ID NO: 32)
Cᵢ[HyP]LVNPLCᵢᵢLHP[dE]W[HArg]Cᵢᵢᵢ;
(SEQ ID NO: 33)
Cᵢ[HyP]LVNPLCᵢᵢLEP[dD]W[HArg]Cᵢᵢᵢ;
(SEQ ID NO: 34)
Cᵢ[HyP]LVNPLCᵢᵢLHP[dD]WTCᵢᵢᵢ;
(SEQ ID NO: 35)
Cᵢ[HyP]LVNPLCᵢᵢLEP[dD]WTCᵢᵢᵢ;
(SEQ ID NO: 36)
Cᵢ[HyP]LVNPLCᵢᵢLEP[dA]WTCᵢᵢᵢ;
Cᵢ[HyP]LVNPLCᵢᵢL[3,3-DPA]P[dD]WTCᵢᵢᵢ
(SEQ ID NO: 37; herein referred to as BCY12860);
(SEQ ID NO: 38)
Cᵢ[HyP][Cba]VNPLCᵢᵢLHP[dD]W[HArg]Cᵢᵢᵢ;
(SEQ ID NO: 39)
Cᵢ[HyP][Cba]VNPLCᵢᵢLEP[dD]WTCᵢᵢᵢ;
(SEQ ID NO: 40)
Cᵢ[HyP][Cba]VNPLCᵢᵢL[3,3-DPA]P[dD]WTCᵢᵢᵢ;
(SEQ ID NO: 41)
Cᵢ[HyP]LVNPLCᵢᵢL[3,3-DPA]P[dD]W[HArg]Cᵢᵢᵢ;
(SEQ ID NO: 42)
Cᵢ[HyP]LVNPLCᵢᵢLHP[d]Nal]W[HArg]Cᵢᵢᵢ;
(SEQ ID NO: 43)
Cᵢ[HyP]LVNPLCᵢᵢL[1Nal]P[dD]W[HArg]Cᵢᵢᵢ;
(SEQ ID NO: 44)
Cᵢ[HyP]LVNPLCᵢᵢLEP[d]Nal]WTCᵢᵢᵢ;
Cᵢ[HyP]LVNPLCᵢᵢL[1Nal]P[dD]WTCᵢᵢᵢ
(SEQ ID NO: 45; herein referred to as BCY13119);
(SEQ ID NO: 46)
Cᵢ[HyP][Cba]VNPLCᵢᵢLEP[dA]WTCᵢᵢᵢ;
(SEQ ID NO: 47)
Cᵢ[HyP][hGlu]VNPLCᵢᵢLHP[dD]W[HArg]Cᵢᵢᵢ;
(SEQ ID NO: 48)
Cᵢ[HyP]LVNPLCᵢᵢ[hGlu]HP[dD]W[HArg]Cᵢᵢᵢ;
(SEQ ID NO: 49)
Cᵢ[HyP]LVNPLCᵢᵢL[hGlu]P[dD]W[HArg]Cᵢᵢᵢ;
(SEQ ID NO: 50)
Cᵢ[HyP]LVNPLCᵢᵢLHP[dNle]W[HArg]Cᵢᵢᵢ;
(SEQ ID NO: 51)
Cᵢ[HyP]LVNPLCᵢᵢL[Nle]P[dD]W[HArg]Cᵢᵢᵢ;
(SEQ ID NO: 154)
[MerPro]ᵢ[HyP]LVNPLCᵢᵢL[3,3-DPA]P[dD]WTCᵢᵢᵢ;
(SEQ ID NO: 155)
Cᵢ[HyP]LVNPLCᵢᵢLHP[dD]W[HArg][Cysam]ᵢᵢᵢ;
(SEQ ID NO: 156)
Cᵢ[HyP]LVNPLCᵢᵢL[His3Me]P[dD]W[HArg]Cᵢᵢᵢ;
(SEQ ID NO: 157)
Cᵢ[HyP]LVNPLCᵢᵢL[His1Me]P[dD]W[HArg]Cᵢᵢᵢ;
(SEQ ID NO: 158)
Cᵢ[HyP]LVNPLCᵢᵢL[4ThiAz]P[dD]W[HArg]Cᵢᵢᵢ;
(SEQ ID NO: 159)
Cᵢ[HyP]LVNPLCᵢᵢLFP[dD]W[HArg]Cᵢᵢᵢ;
(SEQ ID NO: 160)
Cᵢ[HyP]LVNPLCᵢᵢL[Thi]P[dD]W[HArg]Cᵢᵢᵢ;
(SEQ ID NO: 161)
Cᵢ[HyP]LVNPLCᵢᵢL[3Thi]P[dD]W[HArg]Cᵢᵢᵢ;
(SEQ ID NO: 162)
Cᵢ[HyP]LVNPLCᵢᵢLNP[dD]W[HArg]Cᵢᵢᵢ;
(SEQ ID NO: 163)
Cᵢ[HyP]LVNPLCᵢᵢLQP[dD]W[HArg]Cᵢᵢᵢ;
and
(SEQ ID NO: 164)
Cᵢ[HyP]LVNPLCᵢᵢL[K(PYA-(Palmitoyl-Glu-LysN₃)]P[dD]
W[HArg]Cᵢᵢᵢ;

wherein [MerPro]ᵢ, Cᵢ, Cᵢᵢ, Cᵢᵢᵢ and [Cysam]ᵢᵢᵢ represent first (i), second (ii) and third (iii) reactive groups which are selected from cysteine, MerPro and Cysam, HyP represents trans-4-hydroxy-L-proline, HArg represents homoarginine, PYA represents 4-pentynoic acid, 3,3-DPA represents 3,3-diphenylalanine, Cba represents β-cyclobutylalanine, 1Nal represents 1-naphthylalanine, hGlu represents homoglutamic acid, Thi represents 2-thienyl-alanine, 4ThiAz represents beta-(4-thiazolyl)-alanine, His1Me represents N1-methyl-L-histidine, His3Me represents N3-methyl-L-histidine, 3Thi represents 3-thienylalanine, Palmitoyl-Glu-LysN₃[PYA] represents:

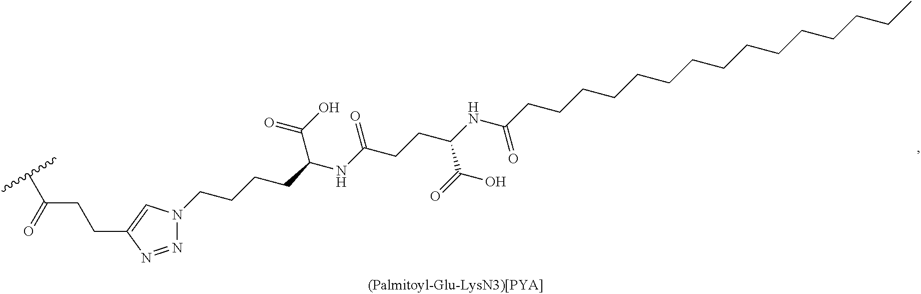 [SMILES: *C(=O)CCc1cn(CCCC[C@H](NC(=O)CC[C@H](NC(=O)CCCCCCCCCCCCCCC)C(=O)O)C(=O)O)nn1]

[K(PYA-(Palmitoyl-Glu-LysN₃)] represents

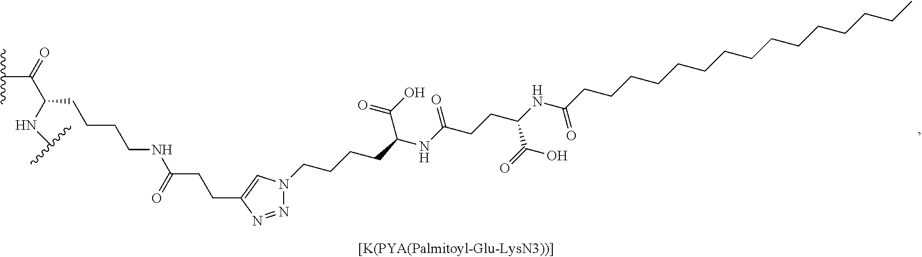 [SMILES: *N[C@@H](CCCCNC(=O)CCc1cn(CCCC[C@H](NC(=O)CC[C@H](NC(=O)CCCCCCCCCCCCCCC)C(=O)O)C(=O)O)nn1)C(*)=O]

Nle represents norleucine, MerPro represents 3-mercaptopropionic acid and Cysam represents cysteamine, or a pharmaceutically acceptable salt thereof.

In some embodiments, an EphA2 binding bicyclic peptide ligand comprises an amino acid sequence which is:

(SEQ ID NO: 24)
Cᵢ[HyP]LVNPLCᵢᵢLHP[dD]W[HArg]Cᵢᵢᵢ;

wherein Cᵢ, Cᵢᵢ, Cᵢᵢᵢ and represent first (i), second (ii) and third (iii) cysteine groups, HyP represents trans-4-hydroxy-L-proline, HArg represents homoarginine, or a pharmaceutically acceptable salt thereof.

In some embodiments, an EphA2 binding bicyclic peptide ligand comprises an amino acid sequence which is:
Cᵢ[HyP]LVNPLCᵢᵢLEP[d1Nal]WTCᵢᵢᵢ(SEQ ID NO: 44);
   wherein Cᵢ, Cᵢᵢ, Cᵢᵢᵢ and represent first (i), second (ii) and third (iii) cysteine groups, HyP represents trans-4-hydroxy-L-proline, 1Nal represents 1-naphthylalanine, or a pharmaceutically acceptable salt thereof.

In some embodiments, an EphA2 binding bicyclic peptide ligand optionally comprises N-terminal and/or C-terminal modifications and comprises:

(SEQ ID NO: 176)
A-[HArg]-D-(SEQ ID NO: 24)
(herein referred to as BCY9594);
(SEQ ID NO: 177)
[B-Ala]-[Sar₁₀]-A-[HArg]-D-(SEQ ID NO: 24)
(herein referred to as BCY6099);
(SEQ ID NO: 178)
[PYA]-A-[HArg]-D-(SEQ NO: 24)
(herein referred to as BCY11813);
(SEQ ID NO: 179)
Ac-A-[HArg]-D-(SEQ ID NO: 24)-[K(PYA)]
(herein referred to as BCY11814);
(SEQ ID NO: 180)
Ac-A-[HArg]-D-(SEQ ID NO: 24)-K
(herein referred to as BCY12734);
(SEQ ID NO: 181)
[NMeAla]-[HArg]-D-(SEQ ID NO: 24)
(herein referred to as BCY13121);
(SEQ ID NO: 182)
[Ac]-(SEQ ID NO: 24)-L[dH]G[dK]
(herein referred to as BCY13125);
(SEQ ID NO: 183)
[PYA]-[B-Ala]-[Sar₁₀]-VGP-(SEQ ID NO: 25)
(herein referred to as BCY8941);
(SEQ ID NO: 184)
Ac-A-[HArg]-D-(SEQ ID NO: 26)
(herein referred to as BCY11815);
(SEQ ID NO: 185)
Ac-A-[HArg]-D-(SEQ ID NO: 27)
(herein referred to as BCY11816);
(SEQ ID NO: 186)
Ac-A-[HArg]-D-(SEQ ID NO: 28)
(herein referred to as BCY11817);
(SEQ ID NO: 187)
Ac-A-[HArg]-D-(SEQ ID NO: 29)
(herein referred to as BCY12735);
(SEQ ID NO: 188)
(Palmitoyl-Glu-LysN₃)[PYA]A[HArg]D-
(SEQ ID NO: 29) (hereinafter known as BCY14327);
(SEQ ID NO: 189)
Ac-A-[HArg]-D-(SEQ ID NO: 30)
(herein referred to as BCY12736);
(SEQ ID NO: 190)
Ac-A-[HArg]-D-(SEQ ID NO: 31)
(herein referred to as BCY12737);
(SEQ ID NO: 191)
A-[HArg]-D-(SEQ ID NO: 32)
(herein referred to as BCY12738);
(SEQ ID NO: 192)
A-[HArg]-E-(SEQ ID NO: 32)
(herein referred to as BCY12739);
(SEQ ID NO: 193)
A-[HArg]-D-(SEQ ID NO: 33)
(herein referred to as BCY12854);
(SEQ ID NO: 194)
A-[HArg]-D-(SEQ ID NO: 34)
(herein referred to as BCY12855);
(SEQ ID NO: 195)
A-[HArg]-D-(SEQ ID NO: 35)
(herein referred to as BCY12856);
(SEQ ID NO: 196)
A-[HArg]-D-(SEQ ID NO: 35)-[dA]
(herein referred to as BCY12857);
(SEQ ID NO: 197)
(SEQ ID NO: 35)-[dA]
(herein referred to as BCY12861);
(SEQ ID NO: 198)
[NMeAla]-[HArg]-D-(SEQ ID NO: 35)
(herein referred to as BCY13122);
(SEQ ID NO: 199)
[dA]-ED-(SEQ ID NO: 35)
(herein referred to as BCY13126);
(SEQ ID NO: 200)
[dA]-[dA]-D-(SEQ ID NO: 35)
(herein referred to as BCY13127);
(SEQ ID NO: 201)
AD-(SEQ ID NO: 35)
(herein referred to as BCY13128);
(SEQ ID NO: 202)
A-[HArg]-D-(SEQ ID NO: 36)
(herein referred to as BCY12858);
(SEQ ID NO: 203)
A-[HArg]-D-(SEQ ID NO: 37)
(herein referred to as BCY12859);
(SEQ ID NO: 204)
Ac-(SEQ ID NO: 37)-[dK]
(herein referred to as BCY13120);
(SEQ ID NO: 205)
A-[HArg]-D-(SEQ ID NO: 38)
(herein referred to as BCY12862);
(SEQ ID NO: 206)
A-[HArg]-D-(SEQ ID NO: 39)
(herein referred to as BCY12863);
(SEQ ID NO: 207)
[dA]-[HArg]-D-(SEQ ID NO: 39)-[dA]
(herein referred to as BCY12864);
(SEQ ID NO: 208)
(SEQ ID NO: 40)-[dA]
(herein referred to as BCY12865);
(SEQ ID NO: 209)
A-[HArg]-D-(SEQ ID NO: 41)
(herein referred to as BCY12866);
(SEQ ID NO: 210)
A-[HArg]-D-(SEQ ID NO: 42)
(herein referred to as BCY13116);
(SEQ ID NO: 211)
A-[HArg]-D-(SEQ ID NO: 43)
(herein referred to as BCY13117);
(SEQ ID NO: 212)
A-[HArg]-D-(SEQ ID NO: 44)
(herein referred to as BCY13118);
(SEQ ID NO: 213)
[dA]-[HArg]-D-(SEQ ID NO: 46)-[dA]
(herein referred to as BCY13123);
(SEQ ID NO: 214)
[d1Nal]-[HArg]-D-(SEQ ID NO: 46)-[dA]
(herein referred to as BCY13124);
(SEQ ID NO: 215)
A-[HArg]-D-(SEQ ID NO: 47)
(herein referred to as BCY13130);
(SEQ ID NO: 216)
A-[HArg]-D-(SEQ ID NO: 48)
(herein referred to as BCY13131);
(SEQ ID NO: 217)
A-[HArg]-D-(SEQ ID NO: 49)
(herein referred to as BCY13132);
(SEQ ID NO: 218)
A-[HArg]-D-(SEQ ID NO: 50)
(herein referred to as BCY13134);
(SEQ ID NO: 219)
A-[HArg]-D-(SEQ ID NO: 51)
(herein referred to as BCY13135);
(SEQ ID NO: 220)
(SEQ ID NO: 154)-[dK]
(herein referred to as BCY13129);
(SEQ ID NO: 221)
A[HArg]D-(SEQ ID NO: 155)
(herein referred to as BCY13133);
(SEQ ID NO: 222)
A[HArg]D-(SEQ ID NO: 156)
(herein referred to as BCY13917);
(SEQ ID NO: 223)
A[HArg]D-(SEQ ID NO: 157)
(herein referred to as BCY13918);
(SEQ ID NO: 224)
A[HArg]D-(SEQ ID NO: 158)
(herein referred to as BCY13919);
(SEQ ID NO: 225)
A[HArg]D-(SEQ ID NO: 159)
(herein referred to as BCY13920);
(SEQ ID NO: 226)
A[HArg]D-(SEQ ID NO: 160)
(herein referred to as BCY13922);
(SEQ ID NO: 227)
A[HArg]D-(SEQ ID NO: 161)
(herein referred to as BCY13923);
(SEQ ID NO: 228)
A[HArg]D-(SEQ ID NO: 162)
(herein referred to as BCY14047);
(SEQ ID NO: 229)
A[HArg]D-(SEQ ID NO: 163)
(herein referred to as BCY14048);
and
(SEQ ID NO: 230)

A[HArg]D-(SEQ ID NO: 164)

(herein referred to as BCY14313);

wherein PYA represents 4-pentynoic acid, B-Ala represents beta-alanine, Sar₁₀ represents 10 sarcosine units, HArg represents homoarginine, NMeAla represents N-methyl-alanine, 1Nal represents 1-naphthylalanine, Palmitoyl-Glu-LysN₃[PYA] represents:

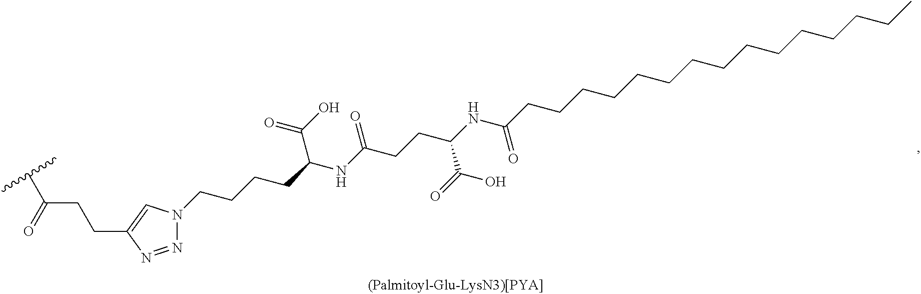 [SMILES: *C(=O)CCc1cn(CCCC[C@H](NC(=O)CC[C@H](NC(=O)CCCCCCCCCCCCCCC)C(=O)O)C(=O)O)nn1]

or a pharmaceutically acceptable salt thereof.

In some embodiments, an EphA2 binding bicyclic peptide ligand optionally comprises N-terminal and/or C-terminal modifications and comprises: A-[HArg]-D-(SEQ ID NO: 24) (SEQ ID NO: 176) (herein referred to as BCY9594);

(SEQ ID NO: 176)
A-[HArg]-D-(SEQ ID NO: 24)
(herein referred to as BCY9594);

wherein HArg represents homoarginine, or a pharmaceutically acceptable salt thereof.

In some embodiments, an EphA2 binding bicyclic peptide ligand optionally comprises N-terminal and/or C-terminal modifications and comprises: A-[HArg]-D-(SEQ ID NO: 44) (SEQ ID NO: 212) (herein referred to as BCY13118);

(SEQ ID NO: 212)
A-[HArg]-D-(SEQ ID NO: 44)
(herein referred to as BCY13118);

wherein HArg represents homoarginine, or a pharmaceutically acceptable salt thereof.

In some embodiments, an EphA2 binding bicyclic peptide ligand comprises an amino acid sequence:

(SEQ ID NO: 24)
Cᵢ[HyP]LVNPLCᵢᵢLHP[dD]W[HArg]Cᵢᵢᵢ;
and
(SEQ ID NO: 25)
CᵢLWDPTPCᵢᵢANLHL[HArg]Cᵢᵢᵢ;

wherein Cᵢ, Cᵢᵢ and Cᵢᵢᵢ represent first, second and third cysteine residues, respectively, HyP represents hydroxyproline, dD represents aspartic acid in D-configuration and HArg represents homoarginine, or a pharmaceutically acceptable salt thereof.

In some embodiments, an EphA2 binding bicyclic peptide ligand comprises an amino acid sequence:

(SEQ ID NO: 24)
Cᵢ[HyP]LVNPLCᵢᵢLHP[dD]W[HArg]Cᵢᵢᵢ;

wherein Cᵢ, Cᵢᵢ and Cᵢᵢᵢ represent first, second and third cysteine residues, respectively, HyP represents hydroxyproline, dD represents aspartic acid in D-configuration and HArg represents homoarginine, or a pharmaceutically acceptable salt thereof.

In some embodiments, an EphA2 binding bicyclic peptide ligand comprises N-terminal modifications and comprises:

(SEQ ID NO: 231)
A-HArg-D-(SEQ ID NO: 24)
(hereinafter referred to as BCY9594);
(SEQ ID NO: 177)
[B-Ala]-[Sar₁₀]-A-[HArg]-D-(SEQ ID NO: 24)
(hereinafter referred to as BCY6099);
(SEQ ID NO: 232)
[PYA]-[B-Ala]-[Sar₁₀]-A-[HArg]-D-(SEQ ID NO: 24)
(hereinafter referred to as BCY6169);
and
(SEQ ID NO: 183)
[PYA]-[B-Ala]-[Sar₁₀]-VGP-(SEQ ID NO: 25)
(hereinafter referred to as BCY8941);

wherein HArg represents homoarginine, PYA represents 4-pentynoic acid, Sar₁₀ represents 10 sarcosine units, B-Ala represents beta-alanine, or a pharmaceutically acceptable salt thereof.

In some embodiments, an EphA2 binding bicyclic peptide ligand comprises N-terminal modifications and comprises:

(SEQ ID NO: 231)
A-HArg-D-(SEQ ID NO: 24)
(hereinafter referred to as BCY9594).

wherein HArg represents homoarginine, or a pharmaceutically acceptable salt thereof.

In some embodiments, the component present on a cancer cell is PD-L1.

Programmed cell death 1 ligand 1 (PD-L1) is a 290 amino acid type I transmembrane protein encoded by the CD274 gene on mouse chromosome 19 and human chromosome 9. PD-L1 expression is involved in evasion of immune responses involved in chronic infection, e.g., chronic viral infection (including, for example, HIV, HBV, HCV and HTLV, among others), chronic bacterial infection (including, for example, Helicobacter pylori, among others), and chronic parasitic infection (including, for example, Schistosoma mansoni). PD-L1 expression has been detected in a number of tissues and cell types including T-cells, B-cells, macrophages, dendritic cells, and nonhaematopoietic cells including endothelial cells, hepatocytes, muscle cells, and placenta.

PD-L1 expression is also involved in suppression of anti-tumor immune activity. Tumors express antigens that can be recognised by host T-cells, but immunologic clearance of tumors is rare. Part of this failure is due to immune suppression by the tumor microenvironment. PD-L1 expression on many tumors is a component of this suppressive milieu and acts in concert with other immunosuppressive signals. PD-L1 expression has been shown in situ on a wide variety of solid tumors including breast, lung, colon, ovarian, melanoma, bladder, liver, salivary, stomach, gliomas, thyroid, thymic epithelial, head, and neck (Brown J A et al. 2003 Immunol. 170:1257-66; Dong H et al. 2002 Nat. Med. 8:793-800; Hamanishi J, et al. 2007 Proc. Natl. Acad. Sci. USA 104:3360-65; Strome S E et al. 2003 Cancer Res. 63:6501-5; Inman B A et al. 2007 Cancer 109:1499-505; Konishi J et al. 2004 Clin. Cancer Res. 10:5094-100; Nakanishi J et al. 2007 Cancer Immunol. Immunother. 56:1173-82; Nomi T et al. 2007 Clin. Cancer Res. 13:2151-57; Thompson R H et al. 2004 Proc. Natl. Acad. Sci. USA 101: 17174-79; Wu C et al. 2006 Acta Histochem. 108:19-24). In addition, the expression of the receptor for PD-L1, Programmed cell death protein 1 (also known as PD-1 and CD279) is upregulated on tumor infiltrating lymphocytes, and this also contributes to tumor immunosuppression (Blank C et al. 2003 Immunol. 171:4574-81). Most importantly, studies relating PD-L1 expression on tumors to disease outcome show that PD-L1 expression strongly correlates with unfavourable prognosis in kidney, ovarian, bladder, breast, gastric, and pancreatic cancer (Hamanishi J et al. 2007 Proc. Natl. Acad. Sci. USA 104:3360-65; Inman B A et al. 2007 Cancer 109:1499-505; Konishi J et al. 2004 Clin. Cancer Res. 10:5094-100; Nakanishi J et al. 2007 Cancer Immunol. Immunother. 56:1173-82; Nomi T et al. 2007 Clin. Cancer Res. 13:2151-57; Thompson R H et al. 2004 Proc. Natl. Acad. Sci. USA 101:17174-79; Wu C et al. 2006 Acta Histochem. 108:19-24). In addition, these studies suggest that higher levels of PD-L1 expression on tumors may facilitate advancement of tumor stage and invasion into deeper tissue structures.

The PD-1 pathway can also play a role in haematologic malignancies. PD-L1 is expressed on multiple myeloma cells but not on normal plasma cells (Liu J et al. 2007 Blood 110:296-304). PD-L1 is expressed on some primary T-cell lymphomas, particularly anaplastic large cell T lymphomas (Brown J A et al, 2003 Immunol. 170:1257-66). PD-1 is highly expressed on the T-cells of angioimmunoblastic lymphomas, and PD-L1 is expressed on the associated follicular dendritic cell network (Dorfman D M et al. 2006 Am. J. Surg. Pathol. 30:802-10). In nodular lymphocyte-predominant Hodgkin lymphoma, the T-cells associated with lymphocytic or histiocytic (L&H) cells express PD-1. Microarray analysis using a readout of genes induced by PD-1 ligation suggests that tumor-associated T-cells are responding to PD-1 signals in situ in Hodgkin lymphoma (Chemnitz J M et al. 2007 Blood 110:3226-33). PD-1 and PD-L1 are expressed on CD4 T-cells in HTLV-1-mediated adult T-cell leukaemia and lymphoma (Shimauchi T et al. 2007 Int. J. Cancer 121: 2585-90). These tumor cells are hyporesponsive to TCR signals.

Studies in animal models demonstrate that PD-L1 on tumors inhibits T-cell activation and lysis of tumor cells and in some cases leads to increased tumor-specific T-cell death (Dong H et al. 2002 Nat. Med. 8:793-800; Hirano F et al. 2005 Cancer Res. 65:1089-96). Tumor-associated APCs can also utilise the PD-1:PD-L1 pathway to control antitumor T-cell responses. PD-L1 expression on a population of tumor-associated myeloid DCs is upregulated by tumor environmental factors (Curiel T J et al. 2003 Nat. Med. 9:562-67). Plasmacytoid dendritic cells (DCs) in the tumor-draining lymph node of B16 melanoma express IDO, which strongly activates the suppressive activity of regulatory T-cells. The suppressive activity of IDO-treated regulatory T-cells required cell contact with IDO-expressing DCs (Sharma M D et al. 2007 Clin. Invest. 117:2570-82).

In some embodiments, the first peptide ligand comprises a PD-L1 binding bicyclic peptide ligand.

In some embodiments, a PD-L1 binding bicyclic peptide ligand is selected from those disclosed in WO 2020/128526 and WO 2020/128527, the contents of each of which are incorporated herein by reference in their entireties.

In some embodiments, a PD-L1 binding bicyclic peptide ligand comprises an amino acid sequence selected from:

(SEQ ID NO: 52)
CᵢSAGWLTMCᵢᵢQKLHLCᵢᵢᵢ;
(SEQ ID NO: 53)
CᵢSAGWLTMCᵢᵢQ[K(PYA)]LHLCiii;
(SEQ ID NO: 54)
CᵢSKGWLTMCᵢᵢQ[K(Ac)]LHLCiii;
(SEQ ID NO: 55)
CᵢSAGWLTKCᵢᵢQ[K(Ac)]LHLCiii;
(SEQ ID NO: 56)
CᵢSAGWLTMCᵢᵢK[K(Ac)]LHLCiii;
(SEQ ID NO: 57)
CᵢSAGWLTMCᵢᵢQ[K(Ac)]LKLCili;
(SEQ ID NO: 58)
CᵢSAGWLTMCᵢᵢQ[HArg]LHLCiii;
and
(SEQ ID NO: 59)
CiSAGWLTMCᵢᵢ[HArg]QLNLCiii;

wherein Cᵢ, Cᵢᵢ and Cᵢᵢᵢ represent first, second and third cysteine residues, respectively, PYA represents 4-pentynoic acid and HArg represents homoarginine, or a pharmaceutically acceptable salt thereof.

In some embodiments, a PD-L1 binding bicyclic peptide ligand optionally comprises N-terminal and/or C-terminal modifications and comprises:

(SEQ ID NO: 233)
[PYA]-[B-Ala]-[Sar₁₀]-SDK-(SEQ ID NO: 52)
(herein referred to as BCY10043);
(SEQ ID NO: 234)
Ac-D-[HArg]-(SEQ ID NO: 52)-PSH
(herein referred to as BCY11865);
(SEQ ID NO: 235)
Ac-SDK-(SEQ ID NO: 53)
(herein referred to as BCY11013);
(SEQ ID NO: 236)
Ac-SDK-(SEQ ID NO: 53)-PSH
(herein referred to as BCY10861);
(SEQ ID NO: 237)
Ac-D-[HArg]-(SEQ ID NO: 54)-PSH
(herein referred to as BCY11866);
(SEQ ID NO: 238)
Ac-D-[HArg]-(SEQ ID NO: 55)-PSH
(herein referred to as BCY11867);
(SEQ ID NO: 239)
Ac-D-[HArg]-(SEQ ID NO: 56)-PSH
(herein referred to as BCY11868);
(SEQ ID NO: 240)
Ac-D-[HArg]-(SEQ ID NO: 57)-PSH
(herein referred to as BCY11869);
(SEQ ID NO: 241)
Ac-SD-[HArg]-(SEQ ID NO: 58)-PSHK
(herein referred to as BCY12479);
and
(SEQ ID NO: 242)
Ac-SD-[HArg]-(SEQ ID NO: 59)-PSHK
(herein referred to as BCY12477);

wherein PYA represents 4-pentynoic acid, B-Ala represents beta-alanine, Sar₁₀ represents 10 sarcosine units and HArg represents homoarginine, or a pharmaceutically acceptable salt thereof.

In some embodiments, a PD-L1 binding bicyclic peptide ligand comprises an amino acid sequence selected from:
Cᵢ[HArg]DWCᵢᵢHWTFSHGHPCGᵢᵢ(SEQ ID NO: 82);
CᵢSAGWLTMCᵢᵢQKLHLCᵢᵢᵢ (SEQ ID NO: 52); and
CᵢSAGWLTMCᵢᵢQ[K(PYA)]LHLCᵢᵢ(SEQ ID NO: 53);
   wherein Cᵢ, Cᵢᵢ and Cᵢᵢᵢ represent first, second and third cysteine residues, respectively, HArg represents homoarginine and PYA represents 4-pentynoic acid, or a pharmaceutically acceptable salt thereof.

In some embodiments, a PD-L1 binding bicyclic peptide ligand comprises N-terminal and/or C-terminal modifications and comprises:

(SEQ ID NO: 243)
[PYA]-[B-Ala]-[Sar₁₀]-(SEQ ID NO: 82)
(hereinafter referred to as BCY8938);
(SEQ ID NO: 233)
[PYA]-[B-Ala]-[Sar₁₀]-SDK-(SEQ ID NO: 52)
(hereinafter referred to as BCY10043);
(SEQ ID NO: 244)
NH₂-SDK-(SEQ ID NO: 52)-[Sar₁₀]-[K(PYA)]
(hereinafter referred to as BCY10044);
(SEQ ID NO: 245)
NH₂-SDK-(SEQ ID NO: 53)
(hereinafter referred to as BCY10045);
and
(SEQ ID NO: 236)
Ac-SDK-(SEQ ID NO: 53)-PSH
(hereinafter referred to as BCY10861);

wherein PYA represents 4-pentynoic acid, B-Ala represents beta-alanine, Sar₁₀ represents 10 sarcosine units, or a pharmaceutically acceptable salt thereof.

In some embodiments, the component present on a cancer cell is prostate-specific membrane antigen (PSMA).

Prostate-specific membrane antigen (PSMA) (also known as Glutamate carboxypeptidase II (GCPII), N-acetyl-L-aspartyl-L-glutamate peptidase I (NAALADase I) and NAAG peptidase) is an enzyme that in humans is encoded by the FOLH1 (folate hydrolase 1) gene. Human GCPII contains 750 amino acids and weighs approximately 84 kDa.

Human PSMA is highly expressed in the prostate, roughly a hundred times greater than in most other tissues. In some prostate cancers, PSMA is the second-most upregulated gene product, with an 8- to 12-fold increase over levels in noncancerous prostate cells. Because of this high expression, PSMA is being developed as potential biomarker for therapy and imaging of some cancers. In human prostate cancer, the higher expressing tumors are associated with quicker time to progression and a greater percentage of patients suffering relapse.

In some embodiments, the first peptide ligand comprises a PSMA binding bicyclic peptide ligand.

In some embodiments, a PSMA binding bicyclic peptide ligand is selected from those disclosed in WO 2019/243455 and WO 2020/120980, the contents of each of which are incorporated herein by reference in their entireties.

In some embodiments, the component present on a cancer cell is membrane type 1 metalloprotease (MT1-MMP).

In some embodiments, the first peptide ligand comprises an MT1-MMP binding bicyclic peptide ligand.

In some embodiments, an MT1-MMP binding bicyclic peptide ligand is selected from those disclosed in WO 2016/067035, WO 2017/191460, and WO 2018/115204, the contents of each of which are incorporated herein by reference in their entireties.

### CD137 Binding Peptide Ligand(s)

CD137 is a member of the tumor necrosis factor (TNF) receptor family. Its alternative names are tumor necrosis factor receptor superfamily member 9 (TNFRSF9), 4-1BB and induced by lymphocyte activation (ILA). CD137 can be expressed by activated T cells, but to a larger extent on CD8+ than on CD4+ T cells. In addition, CD137 expression is found on dendritic cells, follicular dendritic cells, natural killer cells, granulocytes and cells of blood vessel walls at sites of inflammation. One characterized activity of CD137 is its costimulatory activity for activated T cells. Crosslinking of CD137 enhances T cell proliferation, IL-2 secretion, survival and cytolytic activity. Further, it can enhance immune activity to eliminate tumors in mice.

CD137 is a T-cell costimulatory receptor induced on TCR activation (Nam et al., Curr. Cancer Drug Targets, 5:357-363 (2005); Waits et al., Annu. Rev, Immunol., 23:23-68 (2005)). In addition to its expression on activated CD4+ and CD8+ T cells, CD137 is also expressed on CD4+CD25+ regulatory T cells, natural killer (NK) and NK-T cells, monocytes, neutrophils, and dendritic cells. Its natural ligand, CD137L, has been described on antigen-presenting cells including B cells, monocyte/macrophages, and dendritic cells (Watts et al. Annu. Rev. Immunol, 23:23-68 (2005)). On interaction with its ligand, CD137 leads to increased TCR-induced T-cell proliferation, cytokine production, functional maturation, and prolonged CD8+ T-cell survival (Nam et al, Curr. Cancer Drug Targets, 5:357-363 (2005), Watts et d-l., Annu. Rev. Immunol, 23:23-68 (2005)).

Signalling through CD137 by either CD137L or agonistic monoclonal antibodies (mAbs) against CD137 leads to increased TCR-induced T cell proliferation, cytokine production and functional maturation, and prolonged CD8+ T cell survival. These effects result from: (1) the activation of the NF-κB, c-Jun NH2-terminal kinase/stress-activated protein kinase (JNK/SAPK), and p38 mitogen-activated protein kinase (MAPK) signalling pathways, and (2) the control of anti-apoptotic and cell cycle-related gene expression.

Experiments performed in both CD137 and CD137L-deficient mice have additionally demonstrated the importance of CD137 costimulation in the generation of a fully competent T cell response.

IL-2 and IL-15 activated NK cells express CD137, and ligation of CD137 by agonistic mAbs stimulates NK cell proliferation and IFN-γ secretion, but not their cytolytic activity.

Furthermore, CD137-stimulated NK cells promote the expansion of activated T cells in vitro.

In accordance with their costimulatory function, agonist mAbs against CD137 have been shown to promote rejection of cardiac and skin allografts, eradicate established tumors, broaden primary antiviral CD8+ T cell responses, and increase T cell cytolytic potential. These studies support the view that CD137 signalling promotes T cell function which may enhance immunity against tumors and infection.

In some embodiments, where a heterotandem bicyclic peptide complex comprises two or more CD137 binding peptide ligands, two or more of said CD137 binding peptide ligands have the same peptide sequence. In some embodiments, where a heterotandem bicyclic peptide complex comprises two or more CD137 binding peptide ligands, two or more of said CD137 binding peptide ligands have different peptide sequences. In some embodiments, where a heterotandem bicyclic peptide complex comprises two or more CD137 binding peptide ligands, two or more of said CD137 binding peptide ligands are identical. In some embodiments, where a heterotandem bicyclic peptide complex comprises two or more CD137 binding peptide ligands, two or more of said CD137 binding peptide ligands are different.

In some embodiments, where a heterotandem bicyclic peptide complex comprises one CD137 binding peptide ligand, the CD137 binding peptide ligand is a CD137 binding bicyclic peptide ligand. In some embodiments, where a heterotandem bicyclic peptide complex comprises two or more CD137 binding peptide ligands, two or more of said CD137 binding peptide ligands are CD137 binding bicyclic peptide ligands.

In some embodiments, a CD137 binding bicyclic peptide ligand is selected from those disclosed in WO 2019/025811. In some embodiments, where a heterotandem bicyclic peptide complex comprises one CD137 binding peptide ligand, the CD137 binding peptide ligand is a CD137 binding bicyclic peptide ligand selected from those disclosed in WO 2019/025811. In some embodiments, where a heterotandem bicyclic peptide complex comprises two or more CD137 binding peptide ligands, two or more of said CD137 binding bicyclic peptide ligands are independently selected from those disclosed in WO 2019/025811. The contents of WO 2019/025811 are incorporated herein by reference in their entireties.

In some embodiments, a CD137 binding bicyclic peptide ligand comprises an amino acid sequence:

(SEQ ID NO: 5)
CᵢᵢEEGQYCᵢᵢFADPY[Nle]Cᵢᵢᵢ;
(SEQ ID NO: 6)
Cᵢ[tBuAla]PE[D-Ala]PYCᵢᵢFADPY[Nle]Cᵢᵢ;
(SEQ ID NO: 7)
CᵢᵢEEGQYCᵢᵢF[D-Ala]DPY[Nle]Cᵢᵢᵢ;
(SEQ ID NO: 8)
Cᵢ[tBuAla]PK[D-Ala]PYCᵢᵢFADPY[Nle]Cᵢᵢᵢ;
(SEQ ID NO: 9)
Cᵢ[tBuAla]PE[D-Lys]PYCᵢᵢFADPY[Nle]Cᵢᵢᵢ;
(SEQ ID NO: 10)
Cᵢ[tBuAla]P[K(PYA)][D-Ala]PYCᵢᵢFADPY[Nle]Cᵢᵢ;
(SEQ ID NO: 11)
Cᵢ[tBuAla]PE[D-Lys(PYA)]PYCᵢᵢFADPY[Nle]Cᵢᵢ;
(SEQ ID NO: 12)
CᵢᵢEE[D-Lys(PYA)]QYCᵢᵢFADPY(Nle)Cᵢᵢᵢ;
(SEQ ID NO: 60)
Cᵢ[tBuAla]PE[dK]PYCᵢᵢFADPY[Nle]Cᵢᵢ;
(SEQ ID NO: 61)
CᵢᵢEE[dK(PYA)]QYCᵢᵢFADPY[Nle]Cᵢᵢᵢ;
(SEQ ID NO: 62)
Cᵢ[tBuAla]EE(dK)PYCᵢᵢFADPY[Nle]Cᵢᵢᵢ;
(SEQ ID NO: 63)
Cᵢ[tBuAla]PE[dK(PYA)]PYCᵢᵢFADPY[Nle]Cᵢᵢᵢ;
(SEQ ID NO: 64)
Cᵢ[tBuAla]EE[dK(PYA)]PYCᵢᵢFADPY[Nle]Cᵢᵢᵢ;
(SEQ ID NO: 65)
Cᵢ[tBuAla]PE[dK(PYA)]PYCᵢᵢFANPY[Nle]Cᵢᵢᵢ;
(SEQ ID NO: 66)
Cᵢ[tBuAla]PE[dK(PYA)]PYCᵢᵢFAEPY[Nle]Cᵢᵢ;
(SEQ ID NO: 67)
Cᵢ[tBuAla]PE[dK(PYA)]PYCᵢᵢFA[Aad]PY[Nle]Cᵢᵢᵢ;
(SEQ ID NO: 68)
Cᵢ[tBuAla]PE[dK(PYA)]PYCᵢᵢFAQPY[Nle]Cᵢᵢᵢ;
(SEQ ID NO: 69)
Cᵢ[tBuAla]PE[dK(PYA)]PYCᵢᵢFADPY[Nle][Cysam]ᵢᵢᵢ;
(SEQ ID NO: 70; herein referred to as BCY12353)
[MerPro]ᵢ[tBuAla]PE[dK(PYA)]PYCᵢᵢFADPY[Nle]Cᵢᵢᵢ;
(SEQ ID NO: 71; herein referred to as BCY12354)
[MerPro]ᵢ[tBuAla]PE[dK(PYA)]PYCᵢᵢFADPY
[Nle][Cysam]ᵢᵢᵢ;
(SEQ ID NO: 72)
Cᵢ[tBuAla]PE[dK(PYA)]PYCᵢᵢFADPY[Nle]Cᵢᵢᵢ;
(SEQ ID NO: 73)
Cᵢ[tBuAla]PE[dK(PYA)]PYCᵢᵢFADPY[Nle]Cᵢᵢᵢ;
(SEQ ID NO: 74; herein referred to as BCY12372)
Cᵢ[tBuAla]PE[dK(PYA)]PYCᵢᵢFADPY[Nle]Cᵢᵢᵢ;
(SEQ ID NO: 75)
Cᵢ[tBuAla]PE[dK(PYA)]PYCᵢᵢFAD[NMeAla]Y[Nle]Cᵢᵢᵢ;
(SEQ ID NO: 76)
Cᵢ[tBuAla]PE[dK(PYA)]PYCᵢᵢFAD[NMeDAla]Y[Nle]Cᵢᵢᵢ;
(SEQ ID NO: 77)
Cᵢ[tBuAla]P[K(PYA)][dA]PYCᵢᵢFADPY[Nle]Cᵢᵢᵢ;
(SEQ ID NO: 78)
Cᵢ[tBuAla]PE[dK(PYA)]PYCᵢᵢFADPY[Nle]Cᵢᵢᵢ;
(SEQ ID NO: 79)
Cᵢ[tBuAla]PE[dK(Me,PYA)]PYCᵢᵢFADPY[Nle]Cᵢᵢᵢ;
(SEQ ID NO: 80)
Cᵢ[tBuAla]PE[dK(Me,PYA)]PYCᵢᵢFADPY[Nle]Cᵢᵢ;
and
(SEQ ID NO: 81; herein referred to as BCY13137)
[MerPro]ᵢ[tBuAla]EE[dK]PYCᵢᵢFADPY[Nle]Cᵢᵢᵢ;

wherein [MerPro]ᵢ, Cᵢ, Cᵢᵢ, Cᵢᵢᵢ and [Cysam]ᵢᵢᵢ represent first (i), second (ii) and third (iii) reactive groups which are selected from cysteine, MerPro and Cysam, Ne represents norleucine, tBuAla represents t-butyl-alanine, PYA represents 4-pentynoic acid, Aad represents alpha-L-aminoadipic acid, MerPro represents 3-mercaptopropionic acid and Cysam represents cysteamine, NMeAla represents N-methyl-alanine, or a pharmaceutically acceptable salt thereof.

In some embodiments, a CD137 binding bicyclic peptide ligand comprises an amino acid sequence:

(SEQ ID NO: 5)
CᵢᵢEEGQYCᵢᵢFADPY[Nle]Cᵢᵢᵢ;
(SEQ ID NO: 6)
Cᵢ[tBuAla]PE[D-Ala]PYCᵢᵢFADPY[Nle]Cᵢᵢᵢ;
(SEQ ID NO: 7)
CᵢᵢEEGQYCᵢᵢF[D-Ala]DPY[Nle]Cᵢᵢᵢ;
(SEQ ID NO: 8)
Cᵢ[tBuAla]PK[D-Ala]PYCᵢᵢFADPY[Nle]Cᵢᵢ;
(SEQ ID NO: 9)
Cᵢ[tBuAla]PE[D-Lys]PYCᵢᵢFADPY[Nle]Cᵢᵢᵢ;
(SEQ ID NO: 10)
Cᵢ[tBuAla]P[K(PYA)][D-Ala]PYCᵢᵢFADPY[Nle]Cᵢᵢᵢ;
(SEQ ID NO: 11)
Cᵢ[tBuAla]PE[D-Lys(PYA)]PYCᵢᵢFADPY[Nle]Cᵢᵢᵢ;
and
(SEQ ID NO: 12)
CᵢᵢEE[D-Lys(PYA)]QYCᵢᵢFADPY(Nle)Cᵢᵢᵢ;

wherein Cᵢ, Cᵢᵢ and Cᵢᵢᵢ represent first, second and third cysteine residues, respectively, Nle represents norleucine, tBuAla represents t-butyl-alanine, PYA represents 4-pentynoic acid, or a pharmaceutically acceptable salt thereof.

In some embodiments, a CD137 binding bicyclic peptide ligand comprises an amino acid sequence:

(SEQ ID NO: 11)
Cᵢ[tBuAla]PE[D-Lys(PYA)]PYCᵢᵢFADPY[Nle]Cᵢᵢᵢ;

wherein Cᵢ, Cᵢᵢ and Cᵢᵢᵢ represent first, second and third cysteine residues, respectively, tBuAla represents t-butyl-alanine, PYA represents 4-pentynoic acid, Nle represents norleucine, or a pharmaceutically acceptable salt thereof.

In some embodiments, a CD137 binding bicyclic peptide ligand comprises N- and C-terminal modifications and comprises:

(SEQ ID NO: 246)
Ac-A-(SEQ ID NO: 5)-Dap
(herein referred to as BCY7732);
(SEQ ID NO: 247)
Ac-A-(SEQ ID NO: 5)-Dap(PYA)
(herein referred to as BCY7741);
(SEQ ID NO: 248)
Ac-(SEQ ID NO: 6)-Dap
(herein referred to as BCY9172);
(SEQ ID NO: 249)
Ac-(SEQ ID NO: 6)-Dap(PYA)
(herein referred to as BCY11014);
(SEQ ID NO: 250)
Ac-A-(SEQ ID NO: 7)-Dap
(herein referred to as BCY8045);
(SEQ ID NO: 251)
Ac-(SEQ ID NO: 8)-A
(herein referred to as BCY8919);
(SEQ ID NO: 252)
Ac-(SEQ ID NO: 9)-A
(herein referred to as BCY8920);
(SEQ ID NO: 253)
Ac-(SEQ ID NO: 10)-A
(herein referred to as BCY8927);
(SEQ ID NO: 254)
Ac-(SEQ ID NO: 11)-A
(herein referred to as BCY8928);
(SEQ ID NO: 255)
(SEQ ID NO: 11)-A
(herein referred to as BCY14601);
(SEQ ID NO: 256)
Ac-A-(SEQ ID NO: 12)-A
(herein referred to as BCY7744);
(SEQ ID NO: 257)
Ac-(SEQ ID NO: 60)-Dap(PYA)
(herein referred to as BCY11144);
(SEQ ID NO: 258)
Ac-A-(SEQ ID NO: 61)-K
(herein referred to as BCY11613);
(SEQ ID NO: 259)
Ac-(SEQ ID NO: 62)-Dap(PYA)
(herein referred to as BCY12023);
(SEQ ID NO: 260)
Ac-(SEQ ID NO: 63)
(herein referred to as BCY12149);
(SEQ ID NO: 261)
Ac-(SEQ ID NO: 64)
(herein referred to as BCY12143);
(SEQ ID NO: 262)
Ac-(SEQ ID NO: 65)
(herein referred to as BCY12147);
(SEQ ID NO: 263)
Ac-(SEQ ID NO: 66)
(herein referred to as BCY12145);
(SEQ ID NO: 264)
Ac-(SEQ ID NO: 67)
(herein referred to as BCY12146);
(SEQ ID NO: 265)
Ac-(SEQ ID NO: 68)
(herein referred to as BCY12150);
(SEQ ID NO: 266)
Ac-(SEQ ID NO: 69)
(herein referred to as BCY12352);
(SEQ ID NO: 267)
Ac-(SEQ ID NO: 72)-[1,2-diaminoethane]
(herein referred to a BCY12358);
(SEQ ID NO: 268)
[Palmitic Acid]-[yGlu]-[yGlu]-(SEQ ID NO: 73)
(herein referred to as BCY12360);
(SEQ ID NO: 269)
Ac-(SEQ ID NO: 75)
(herein referred to as BCY12381);
(SEQ ID NO: 270)
Ac-(SEQ ID NO: 76)
(herein referred to as BCY12382);
(SEQ ID NO: 271)
Ac-(SEQ ID NO: 77)-K
(herein referred to as BCY12357);
(SEQ ID NO: 272)
Ac-(SEQ ID NO: 78)-[dA]
(herein referred to as BCY13095);
(SEQ ID NO: 273)
[Ac]-(SEQ ID NO: 78)-K
(herein referred to as BCY13389);
(SEQ ID NO: 274)
Ac-(SEQ ID NO: 79)-[dA]
(herein referred to as BCY13096);
and
(SEQ ID NO: 275)
Ac-(SEQ ID NO: 80)
(herein referred to as BCY13097);

wherein Ac represents an acetyl group, Dap represents diaminopropionic acid and PYA represents 4-pentynoic acid, or a pharmaceutically acceptable salt thereof.

In some embodiments, a CD137 binding bicyclic peptide ligand comprises N- and C-terminal modifications and comprises:

(SEQ ID NO: 246)
Ac-A-(SEQ ID NO: 5)-Dap
(herein referred to as BCY7732);
(SEQ ID NO: 247)
Ac-A-(SEQ ID NO: 5)-Dap(PYA)
(herein referred to as BCY7741);
(SEQ ID NO: 248)
Ac-(SEQ ID NO: 6)-Dap
(herein referred to as BCY9172);
(SEQ ID NO: 249)
Ac-(SEQ ID NO: 6)-Dap(PYA)
(herein referred to as BCY11014);
(SEQ ID NO: 250)
Ac-A-(SEQ ID NO: 7)-Dap
(herein referred to as BCY8045);
(SEQ ID NO: 251)
Ac-(SEQ ID NO: 8)-A
(herein referred to as BCY8919);
(SEQ ID NO: 252)
Ac-(SEQ ID NO: 9)-A
(herein referred to as BCY8920);
(SEQ ID NO: 253)
Ac-(SEQ ID NO: 10)-A
(herein referred to as BCY8927);
(SEQ ID NO: 254)
Ac-(SEQ ID NO: 11)-A
(herein referred to as BCY8928);
and
(SEQ ID NO: 256)
Ac-A-(SEQ ID NO: 12)-A
(herein referred to as BCY7744);

wherein Ac represents an acetyl group, Dap represents diaminopropionic acid and PYA represents 4-pentynoic acid, or a pharmaceutically acceptable salt thereof.

In some embodiments, a CD137 binding bicyclic peptide ligand comprises N- and C-terminal modifications and comprises:

(SEQ ID NO: 254)
Ac-(SEQ ID NO: 11)-A
(herein referred to as BCY8928);

wherein Ac represents an acetyl group, or a pharmaceutically acceptable salt thereof.

In some embodiments, where a heterotandem bicyclic peptide complex comprises two or more CD137 binding peptide ligands, each of said two or more CD137 binding peptide ligands has the same peptide sequence and said peptide sequence comprises Ac-(SEQ ID NO: 11)-A (SEQ ID NO: 254) (herein referred to as BCY8928), wherein Ac represents an acetyl group, or a pharmaceutically acceptable salt thereof.

In some embodiments, where a heterotandem bicyclic peptide complex comprises two CD137 binding peptide ligands, both of said two CD137 binding peptide ligands have the same peptide sequence which comprises Ac-(SEQ ID NO: 11)-A (SEQ ID NO: 254) (herein referred to as BCY8928), wherein Ac represents an acetyl group, or a pharmaceutically acceptable salt thereof.

### Linkers

### Heterotandem Bicyclic Peptide Complex Comprising Two or More CD137 Binding Peptide Ligands

It will be appreciated that the first peptide ligand may be conjugated to the two or more second peptide ligands via any suitable linker. Typically, the design of said linker will be such that the three Bicyclic peptides are presented in such a manner that they can bind unencumbered to their respective targets either alone or while simultaneously binding to both target receptors. Additionally, the linker should permit binding to both targets simultaneously while maintaining an appropriate distance between the target cells that would lead to the desired functional outcome. The properties of the linker may be modulated to increase length, rigidity or solubility to optimise the desired functional outcome. The linker may also be designed to permit the attachment of more than one Bicycle to the same target. Increasing the valency of either binding peptide may serve to increase the affinity of the heterotandem for the target cells or may help to induce oligomerisation of one or both of the target receptors.

In some embodiments, the linker is a branched linker to allow one first peptide at one end and the two or more second peptides at the other end.

In some embodiments, the branched linker is selected from:

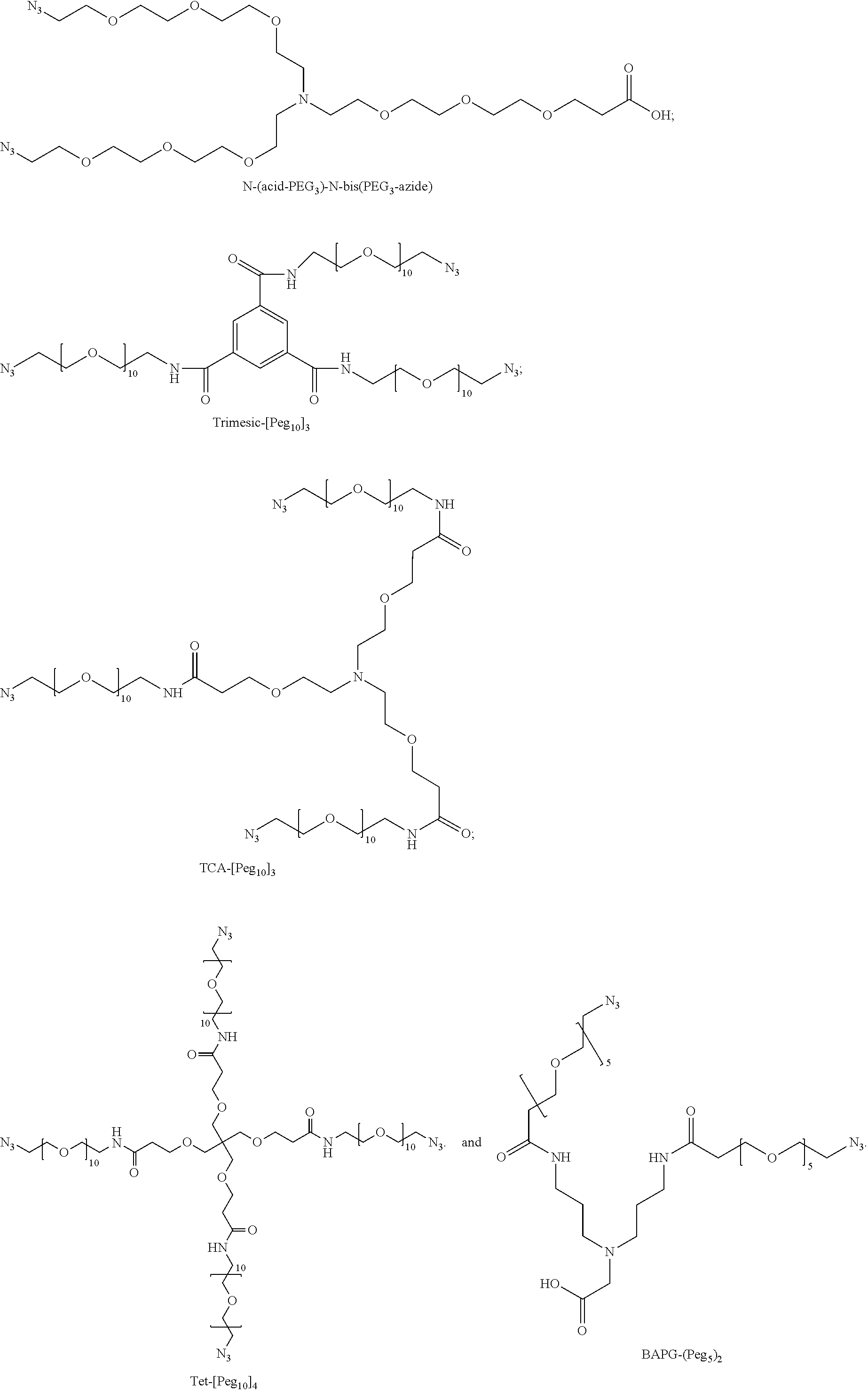

In some embodiments, the branched linker is:

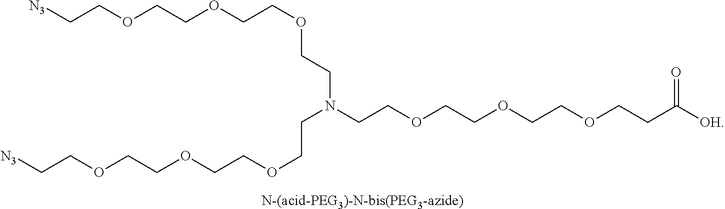 [SMILES: [N-]=[N+]=NCCOCCOCCOCCN(CCOCCOCCOCCN=[N+]=[N-])CCOCCOCCOCCC(=O)O]

### Heterotandem Bicyclic Peptide Complex Comprising One CD137 Binding Peptide Ligand

It will be appreciated that the first peptide ligand may be conjugated to the second peptide ligand via any suitable linker. Typically the design of said linker will be such that the two Bicyclic peptides are presented in such a manner that they can bind unencumbered to their respective targets either alone or while simultaneously binding to both target receptors. Additionally, the linker should permit binding to both targets simultaneously while maintaining an appropriate distance between the target cells that would lead to the desired functional outcome. The properties of the linker may be modulated to increase length, rigidity or solubility to optimise the desired functional outcome. The linker may also be designed to permit the attachment of more than one Bicycle to the same target. Increasing the valency of either binding peptide may serve to increase the affinity of the heterotandem for the target cells or may help to induce oligomerisation of one or both of the target receptors.

In one embodiment, the linker is selected from the following sequences: -PEG₅- and TCA-[PEG₁₀]₃.

Structural representations of these linkers are detailed below:

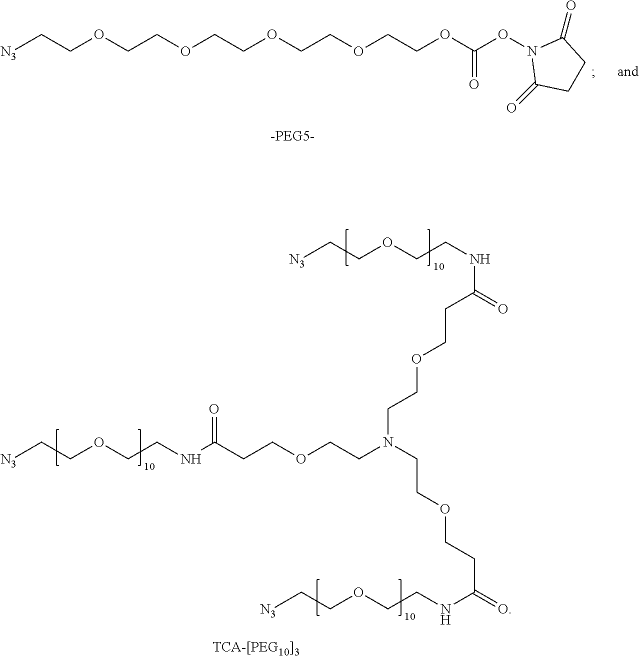

In some embodiments, the linker is selected from the following sequences: —CH₂—, -PEG₅-, -PEG₁₀-, -PEG₁₂-, -PEG₂₃-, -PEG₂₄-, -PEG₁₅-Sar₅- (SEQ ID NO: 276), -PEG₁₀-Sar₁₀-(SEQ ID NO: 177), -PEG₅-Sar₁₅- (SEQ ID NO: 278), -PEG₅-Sar₅- (SEQ ID NO: 279), -B-Ala-Sar₂₀- (SEQ ID NO: 280), -B-Ala-Sar₁₀-PEG₁₀- (SEQ ID NO: 281), -B-Ala-Sar₅-PEG₁₅- (SEQ ID NO: 282) and -B-Ala-Sar₅-PEG₅- (SEQ ID NO: 283).

In some embodiments, the linker is selected from the following. The linkers below are disclosed as SEQ ID NOS 276-283, respectively, in order of appearance:

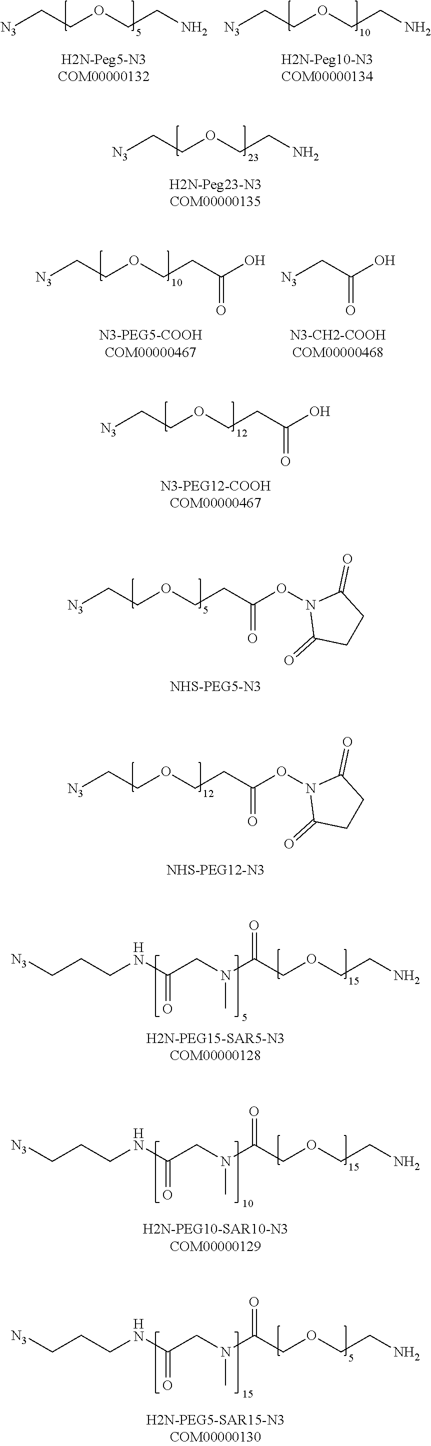

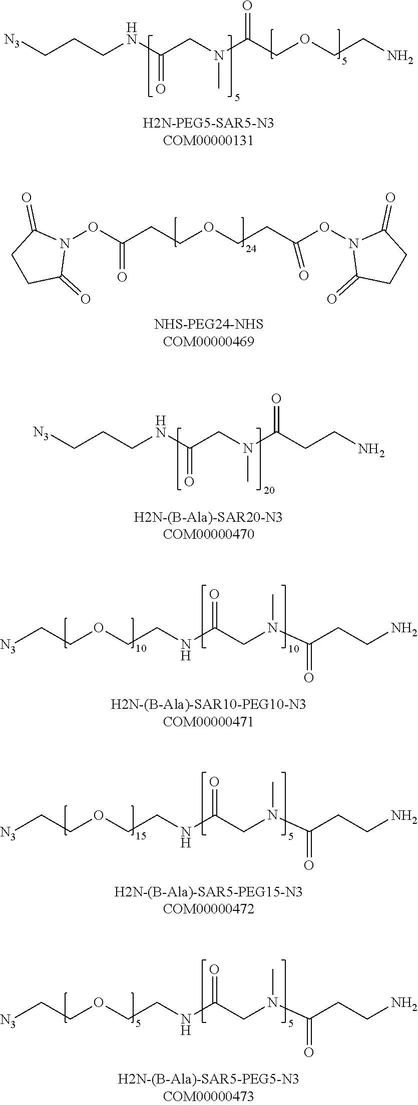

### Heterotandem Bicyclic Peptide Complexes

In some embodiments, where a heterotandem bicyclic peptide complex comprises two or more CD137 binding peptide ligands, the first peptide ligand comprises a Nectin-4 binding bicyclic peptide ligand attached to a TATA scaffold, each of the two or more CD137 binding bicyclic peptide ligands is attached to a TATA scaffold, and said heterotandem bicyclic peptide complex is selected from the complexes listed in Table A:

TABLE A
(Nectin-4:CD137; 1:2)

| Complex | | Attachment | | | Attachment |
|---|---|---|---|---|---|
| No. | Nectin-4 BCY No. | Point | Linker | CD137 BCY No. | Point |
| BCY11863 | BCY8116 | N-terminus | N-(acid- | BCY8928 | dLys (PYA)4 |
| | | | PEG₃)-N- | | |
| | | | bis(PEG₃- | | |
| | | | azide) | | |
| BCY12484 | BCY8116 | N-terminus | N-(acid- | BCY12143 | dLys(PYA)4 |
| | | | PEG₃)-N- | | |
| | | | bis(PEG₃- | | |
| | | | azide) | | |
| BCY10918 | BCY11015 | N-term PYA | Trimesic- | BCY8928 | dLys(PYA)4 |
| | | | [Peg₁₀]₃ | | |
| BCY10919 | BCY11015 | N-term PYA | Trimesic- | BCY11014 | C-term |
| | | | [Peg₁₀]₃ | | Dap(PYA) |
| BCY11027 | BCY11015 | N-term PYA | TCA-[Peg₁₀]₃ | BCY8928 | dLys(PYA)4 |
| BCY11385 | BCY8116 | N-terminus | N-(acid- | BCY11014 | C-term |
| | | | PEG₃)-N- | | Dap(PYA) |
| | | | bis(PEG₃-azide) | | |
| BCY11864 | BCY8116 | N-terminus | N-(acid- | BCY7744 | dLys(PYA)4 |
| | | | PEG₃)-N- | | |
| | | | bis(PEG₃- | | |
| | | | azide) | | |
| BCY12485 | BCY8116 | N-terminus | N-(acid- | BCY12149 | dLys(PYA)4 |
| | | | PEG₃)-N- | | |
| | | | bis(PEG₃- | | |
| | | | azide) | | |
| BCY12486 | BCY8116 | N-terminus | N-(acid- | BCY12147 | dLys(PYA)4 |
| | | | PEG₃)-N- | | |
| | | | bis(PEG₃- | | |
| | | | azide) | | |
| BCY12586 | BCY8116 | N-terminus | N-(acid- | BCY12352 | dLys(PYA)4 |
| | | | PEG₃)-N- | | |
| | | | bis(PEG₃- | | |
| | | | azide) | | |
| BCY12487 | BCY8116 | N-terminus | N-(acid- | BCY12145 | dLys(PYA)4 |
| | | | PEG₃)-N- | | |
| | | | bis(PEG₃- | | |
| | | | azide) | | |
| BCY12490 | BCY12024 | dLys3 | N-(acid- | BCY8928 | dLys(PYA)4 |
| | | | PEG₃)-N- | | |
| | | | bis(PEG₃- | | |
| | | | azide) | | |
| BCY12587 | BCY8116 | N-terminus | N-(acid- | BCY12353 | dLys(PYA)4 |
| | | | PEG₃)-N- | | |
| | | | bis(PEG₃- | | |
| | | | azide) | | |
| BCY12588 | BCY8116 | N-terminus | N-(acid- | BCY12354 | dLys(PYA)4 |
| | | | PEG₃)-N- | | |
| | | | bis(PEG₃- | | |
| | | | azide) | | |
| BCY12589 | BCY12371 | N-terminus | N-(acid- | BCY8928 | dLys(PYA)4 |
| | | | PEG₃)-N- | | |
| | | | bis(PEG₃- | | |
| | | | azide) | | |
| BCY12590 | BCY12384 | N-terminus | N-(acid- | BCY8928 | dLys(PYA)4 |
| | | | PEG₃)-N- | | |
| | | | bis(PEG₃- | | |
| | | | azide) | | |
| BCY12760 | BCY8116 | N-terminus | N-(acid- | BCY12381 | dLys(PYA)4 |
| | | | PEG₃)-N- | | |
| | | | bis(PEG₃- | | |
| | | | azide) | | |
| BCY12761 | BCY8116 | N-terminus | N-(acid- | BCY12382 | dLys(PYA)4 |
| | | | PEG₃)-N- | | |
| | | | bis(PEG₃- | | |
| | | | azide) | | |
| BCY13390 | BCY8116 | N terminus | N-(acid- | BCY8928 | dLys(PYA)4 |
| | | | PEG₃)-N- | BCY13389 | dLys(PYA)4 |
| | | | bis(PEG₃- | | |
| | | | azide) | | |
| BCY14602 | BCY8116 | N terminus | N-(acid- | BCY14601 | dLys(PYA)4 |
| | | | PEG₃)-N- | | |
| | | | bis(PEG₃- | | |
| | | | azide) | | |
| BCY15155 | BCY8116 | N terminus | N-(acid- | BCY14601 | dLys(PYA)4 |
| | | | PEG₃)-N- | BCY8928 | dLys(PYA)4 |
| | | | bis(PEG₃- | | |
| | | | azide) | | |

In some embodiments, the heterotandem bicyclic peptide complex is selected from: BCY11027, BCY11863 and BCY11864. In some embodiments, the heterotandem bicyclic peptide complex is selected from: BCY11863 and BCY11864.

The heterotandem bicyclic peptide complex BCY11863 (also referred to as BT7480) consists of a Nectin-4 specific peptide BCY8116 linked to two CD137 specific peptides (both of which are BCY8928) via a N-(acid-PEG₃)-N-bis(PEG₃-azide) linker, shown pictorially as:

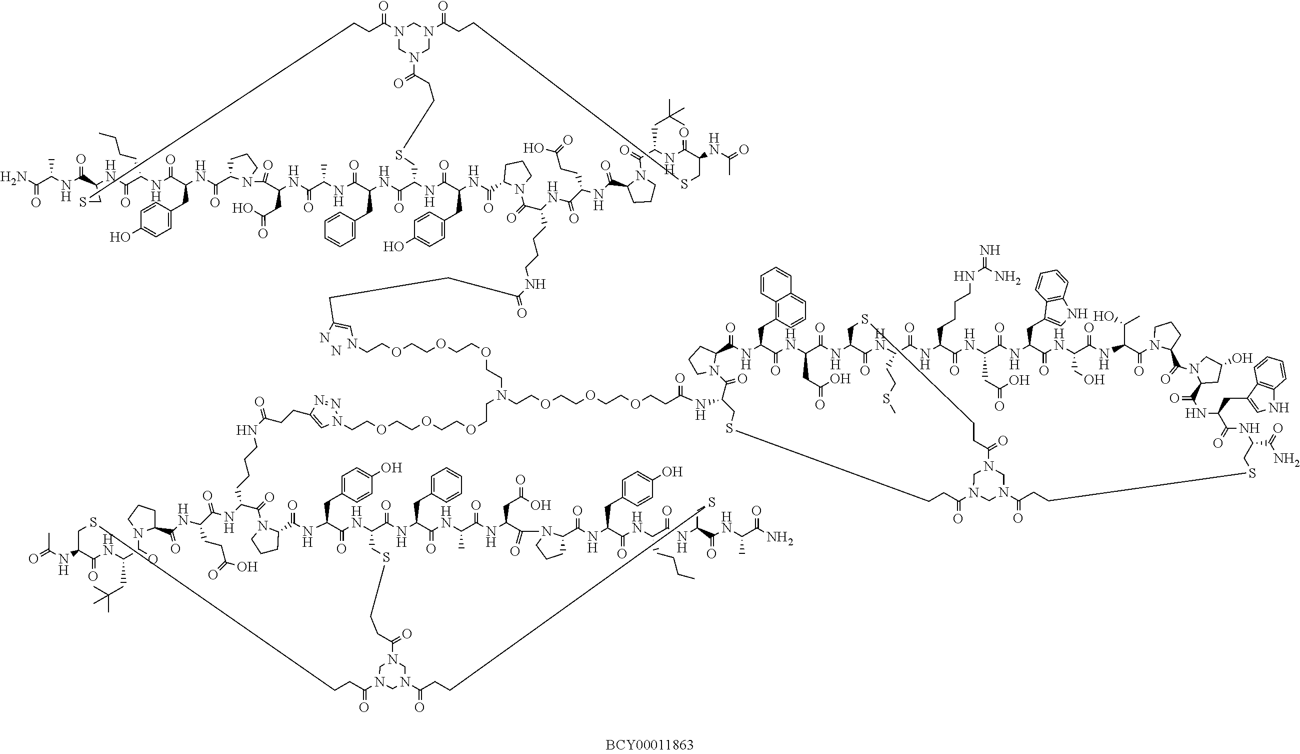

CD137 is a homotrimeric protein and the natural ligand CD137L exists as a homotrimer either expressed on immune cells or secreted. The biology of CD137 is highly dependent on multimerization to induce CD137 activity in immune cells. One way to generate CD137 multimerization is through cellular cross-linking of the CD137 specific agonist through interaction with a specific receptor present on another cell. The advantage of the heterotandem complexes of the present invention is that the presence of two or more peptide ligands specific for an immune cell component, such as CD137, provides a more effective clustering of CD137. For example, it has been found that BCY11863 demonstrated strong CD137 activation and induces robust IL-2 and IFN-γ cytokine secretion, and that BCY11863 demonstrated an excellent PK profile with a terminal half-life of 4.1 hours in SD Rats and 5.3 hours in cyno.

The heterotandem bicyclic peptide complex BCY11027 consists of a Nectin-4 specific peptide BCY11015 linked to two CD137 specific peptides (both of which are BCY8928) via a TCA-[Peg₁₀]₃ linker, shown pictorially as:

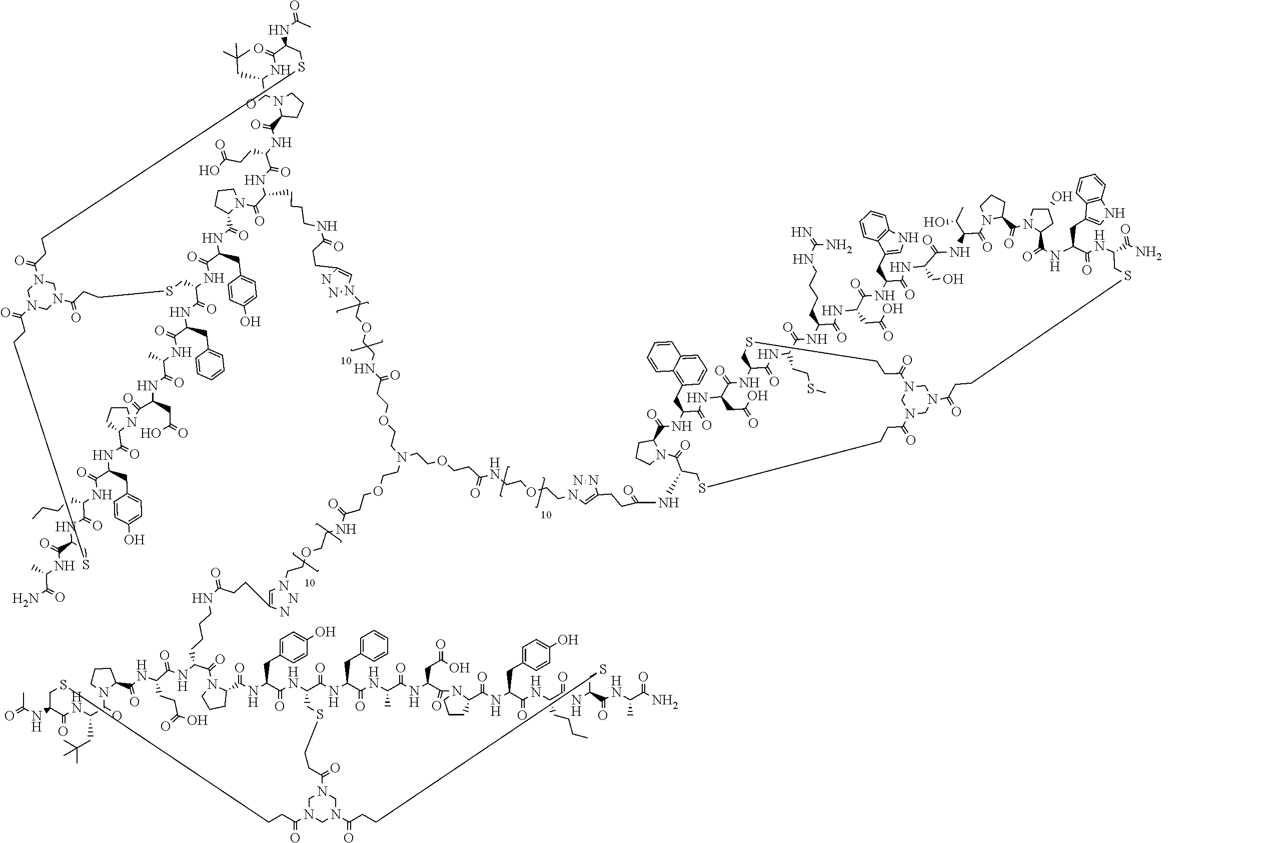

It has been found that Nectin-4/CD137 heterotandem BCY11027 induces target dependent cytokine release in ex vivo cultures of primary patient-derived lung tumors, and induces Nectin-4 dependent change in several immune markers (normalized to vehicle) and in % CD8+ki67+ T cells in patient-derived samples that correlated with the level of Nectin-4 expression.

In some embodiments, where a heterotandem bicyclic peptide complex comprises two or more CD137 binding peptide ligands, the first peptide ligand comprises a Nectin-4 binding bicyclic peptide ligand attached to a TATA scaffold, each of the two or more CD137 binding bicyclic peptide ligands is attached to a TATA scaffold, and said heterotandem bicyclic peptide complex is selected from the complexes listed in Table B:

TABLE B
(Nectin-4:CD137; 1:3)

| Complex | Nectin-4 | Attachment | | CD137 | Attachment |
|---|---|---|---|---|---|
| No. | BCY No. | Point | Linker | BCY No. | Point |
| BCY11021 | BCY11016 | N-term | Tet- | BCY7744 | dLys(PYA)4 |
| | | PYA | [Peg₁₀]₄ | | |
| BCY11022 | BCY11016 | N-term | Tet- | BCY8928 | dLys(PYA)4 |
| | | PYA | [Peg₁₀]₄ | | |

In some embodiments, where a heterotandem bicyclic peptide complex comprises two or more CD137 binding peptide ligands, the first peptide ligand comprises an EphA2 binding bicyclic peptide ligand attached to a TATA scaffold, each of the two or more CD137 binding bicyclic peptide ligands is attached to a TATA scaffold, and said heterotandem bicyclic peptide complex is selected from the complexes listed in Table C:

TABLE C
(EphA2:CD137; 1:2)

| | | | | | Attach- |
|---|---|---|---|---|---|
| Complex | EphA2 | Attachment | | CD137 | ment |
| No. | BCY No. | Point | Linker | BCY No. | Point |
| BCY12491 | BCY9594 | N-terminus | N-(acid- | BCY8928 | dLys |
| | | | PEG₃)-N- | | (PYA)4 |
| | | | bis(PEG₃- | | |
| | | | azide) | | |
| BCY12723 | BCY9594 | N-terminus | N-(acid- | BCY12143 | dLys |
| | | | PEG₃)-N- | | (PYA)4 |
| | | | bis(PEG₃- | | |
| | | | azide) | | |
| BCY12724 | BCY9594 | N-terminus | N-(acid- | BCY12149 | dLys |
| | | | PEG₃)-N- | | (PYA)4 |
| | | | bis(PEG₃- | | |
| | | | azide) | | |
| BCY12725 | BCY9594 | N-terminus | N-(acid- | BCY12147 | dLys |
| | | | PEG₃)-N- | | (PYA)4 |
| | | | bis(PEG₃- | | |
| | | | azide) | | |
| BCY12726 | BCY9594 | N-terminus | N-(acid- | BCY12145 | dLys |
| | | | PEG₃)-N- | | (PYA)4 |
| | | | bis(PEG₃- | | |
| | | | azide) | | |
| BCY12728 | BCY9594 | N-terminus | N-(acid- | BCY12150 | dLys |
| | | | PEG₃)-N- | | (PYA)4 |
| | | | bis(PEG₃- | | |
| | | | azide) | | |
| BCY12729 | BCY9594 | N-terminus | N-(acid- | BCY12352 | dLys |
| | | | PEG₃)-N- | | (PYA)4 |
| | | | bis(PEG₃- | | |
| | | | azide) | | |
| BCY12730 | BCY9594 | N-terminus | N-(acid- | BCY12353 | dLys |
| | | | PEG₃)-N- | | (PYA)4 |
| | | | bis(PEG₃- | | |
| | | | azide) | | |
| BCY12731 | BCY9594 | N-terminus | N-(acid- | BCY12354 | dLys |
| | | | PEG₃)-N- | | (PYA)4 |
| | | | bis(PEG₃- | | |
| | | | azide) | | |
| BCY12732 | BCY9594 | N-terminus | N-(acid- | BCY12360 | dLys |
| | | | PEG₃)-N- | | (PYA)4 |
| | | | bis(PEG₃- | | |
| | | | azide) | | |
| BCY12973 | BCY12734 | C-term Lys | N-(acid- | BCY8928 | dLys |
| | | | PEG₃)-N- | | (PYA)4 |
| | | | bis(PEG₃- | | |
| | | | azide) | | |
| BCY12974 | BCY12735 | Lys8 | N-(acid- | BCY8928 | dLys |
| | | | PEG₃)-N- | | (PYA)4 |
| | | | bis(PEG₃- | | |
| | | | azide) | | |
| BCY12975 | BCY12736 | Lys2 | N-(acid- | BCY8928 | dLys |
| | | | PEG₃)-N- | | (PYA)4 |
| | | | bis(PEG₃- | | |
| | | | azide) | | |
| BCY12976 | BCY12737 | Lys7 | N-(acid- | BCY8928 | dLys |
| | | | PEG₃)-N- | | (PYA)4 |
| | | | bis(PEG₃- | | |
| | | | azide) | | |
| BCY12977 | BCY12738 | N-terminus | N-(acid- | BCY8928 | dLys |
| | | | PEG₃)-N- | | (PYA)4 |
| | | | bis(PEG₃- | | |
| | | | azide) | | |
| BCY12978 | BCY12739 | N-terminus | N-(acid- | BCY8928 | dLys |
| | | | PEG₃)-N- | | (PYA)4 |
| | | | bis(PEG₃- | | |
| | | | azide) | | |
| BCY12979 | BCY9594 | N-terminus | BAPG- | BCY8928 | dLys |
| | | | (Peg₅)₂ | | (PYA)4 |
| BCY13042 | BCY12854 | N-terminus | N-(acid- | BCY8928 | dLys |
| | | | PEG₃)-N- | | (PYA)4 |
| | | | bis(PEG₃- | | |
| | | | azide) | | |
| BCY13043 | BCY12855 | N-terminus | N-(acid- | BCY8928 | dLys |
| | | | PEG₃)-N- | | (PYA)4 |
| | | | bis(PEG₃- | | |
| | | | azide) | | |
| BCY13044 | BCY12856 | N-terminus | N-(acid- | BCY8928 | dLys |
| | | | PEG₃)-N- | | (PYA)4 |
| | | | bis(PEG₃- | | |
| | | | azide) | | |
| BCY13045 | BCY12857 | N-terminus | N-(acid- | BCY8928 | dLys |
| | | | PEG₃)-N- | | (PYA)4 |
| | | | bis(PEG₃- | | |
| | | | azide) | | |
| BCY13046 | BCY12858 | N-terminus | N-(acid- | BCY8928 | dLys |
| | | | PEG₃)-N- | | (PYA)4 |
| | | | bis(PEG₃- | | |
| | | | azide) | | |
| BCY13047 | BCY12859 | N-terminus | N-(acid- | BCY8928 | dLys |
| | | | PEG₃)-N- | | (PYA)4 |
| | | | bis(PEG₃- | | |
| | | | azide) | | |
| BCY13048 | BCY12860 | N-terminus | N-(acid- | BCY8928 | dLys |
| | | | PEG₃)-N- | | (PYA)4 |
| | | | bis(PEG₃- | | |
| | | | azide) | | |
| BCY13049 | BCY12861 | N-terminus | N-(acid- | BCY8928 | dLys |
| | | | PEG₃)-N- | | (PYA)4 |
| | | | bis(PEG₃- | | |
| | | | azide) | | |
| BCY13050 | BCY12862 | N-terminus | N-(acid- | BCY8928 | dLys |
| | | | PEG₃)-N- | | (PYA)4 |
| | | | bis(PEG₃- | | |
| | | | azide) | | |
| BCY13051 | BCY12863 | N-terminus | N-(acid- | BCY8928 | dLys |
| | | | PEG₃)-N- | | (PYA)4 |
| | | | bis(PEG₃- | | |
| | | | azide) | | |
| BCY13052 | BCY12864 | N-terminus | N-(acid- | BCY8928 | dLys |
| | | | PEG₃)-N- | | (PYA)4 |
| | | | bis(PEG₃- | | |
| | | | azide) | | |
| BCY13053 | BCY12865 | N-terminus | N-(acid- | BCY8928 | dLys |
| | | | PEG₃)-N- | | (PYA)4 |
| | | | bis(PEG₃- | | |
| | | | azide) | | |
| BCY13054 | BCY12866 | N-terminus | N-(acid- | BCY8928 | dLys |
| | | | PEG₃)-N- | | (PYA)4 |
| | | | bis(PEG₃- | | |
| | | | azide) | | |
| BCY13138 | BCY12856 | N-terminus | N-(acid- | BCY12353 | dLys |
| | | | PEG₃)-N- | | (PYA)4 |
| | | | bis(PEG₃- | | |
| | | | azide) | | |
| BCY13139 | BCY9594 | N-terminus | N-(acid- | BCY13137 | dLys |
| | | | PEG₃)-N- | | (PYA)4 |
| | | | bis(PEG₃- | | |
| | | | azide) | | |
| BCY13140 | BCY12856 | N-terminus | N-(acid- | BCY13137 | dLys |
| | | | PEG₃)-N- | | (PYA)4 |
| | | | bis(PEG₃- | | |
| | | | azide) | | |
| BCY13270 | BCY13116 | N-terminus | N-(acid- | BCY8928 | dLys |
| | | | PEG₃)-N- | | (PYA)4 |
| | | | bis(PEG₃- | | |
| | | | azide) | | |
| BCY13271 | BCY13117 | N-terminus | N-(acid- | BCY8928 | dLys |
| | | | PEG₃)-N- | | (PYA)4 |
| | | | bis(PEG₃- | | |
| | | | azide) | | |
| BCY13272 | BCY13118 | N-terminus | N-(acid- | BCY8928 | dLys |
| | | | PEG₃)-N- | | (PYA)4 |
| | | | bis(PEG₃- | | |
| | | | azide) | | |
| BCY13273 | BCY13119 | N-terminus | N-(acid- | BCY8928 | dLys |
| | | | PEG₃)-N- | | (PYA)4 |
| | | | bis(PEG₃- | | |
| | | | azide) | | |
| BCY13274 | BCY13120 | C-term dLys | N-(acid- | BCY8928 | dLys |
| | | | PEG₃)-N- | | (PYA)4 |
| | | | bis(PEG₃- | | |
| | | | azide) | | |
| BCY13275 | BCY13121 | N-terminus | N-(acid- | BCY8928 | dLys |
| | | | PEG₃)-N- | | (PYA)4 |
| | | | bis(PEG₃- | | |
| | | | azide) | | |
| BCY13276 | BCY13122 | N-terminus | N-(acid- | BCY8928 | dLys |
| | | | PEG₃)-N- | | (PYA)4 |
| | | | bis(PEG₃- | | |
| | | | azide) | | |
| BCY13277 | BCY13123 | N-terminus | N-(acid- | BCY8928 | dLys |
| | | | PEG₃)-N- | | (PYA)4 |
| | | | bis(PEG₃- | | |
| | | | azide) | | |
| BCY13278 | BCY13124 | N-terminus | N-(acid- | BCY8928 | dLys |
| | | | PEG₃)-N- | | (PYA)4 |
| | | | bis(PEG₃- | | |
| | | | azide) | | |
| BCY13280 | BCY13126 | N-terminus | N-(acid- | BCY8928 | dLys |
| | | | PEG₃)-N- | | (PYA)4 |
| | | | bis(PEG₃- | | |
| | | | azide) | | |
| BCY13281 | BCY13127 | N-terminus | N-(acid- | BCY8928 | dLys |
| | | | PEG₃)-N- | | (PYA)4 |
| | | | bis(PEG₃- | | |
| | | | azide) | | |
| BCY13282 | BCY13128 | N-terminus | N-(acid- | BCY8928 | dLys |
| | | | PEG₃)-N- | | (PYA)4 |
| | | | bis(PEG₃- | | |
| | | | azide) | | |
| BCY13284 | BCY13130 | N-terminus | N-(acid- | BCY8928 | dLys |
| | | | PEG₃)-N- | | (PYA)4 |
| | | | bis(PEG₃- | | |
| | | | azide) | | |
| BCY13285 | BCY13131 | N-terminus | N-(acid- | BCY8928 | dLys |
| | | | PEG₃)-N- | | (PYA)4 |
| | | | bis(PEG₃- | | |
| | | | azide) | | |
| BCY13286 | BCY13132 | N-terminus | N-(acid- | BCY8928 | dLys |
| | | | PEG₃)-N- | | (PYA)4 |
| | | | bis(PEG₃- | | |
| | | | azide) | | |
| BCY13288 | BCY13134 | N-terminus | N-(acid- | BCY8928 | dLys |
| | | | PEG₃)-N- | | (PYA)4 |
| | | | bis(PEG₃- | | |
| | | | azide) | | |
| BCY13289 | BCY13135 | N-terminus | N-(acid- | BCY8928 | dLys |
| | | | PEG₃)-N- | | (PYA)4 |
| | | | bis(PEG₃- | | |
| | | | azide) | | |
| BCY13341 | BCY12865 | N-terminus | N-(acid- | BCY12353 | dLys |
| | | | PEG₃)-N- | | (PYA)4 |
| | | | bis(PEG₃- | | |
| | | | azide) | | |
| BCY13343 | BCY12860 | N-terminus | N-(acid- | BCY12353 | dLys |
| | | | PEG₃)-N- | | (PYA)4 |
| | | | bis(PEG₃- | | |
| | | | azide) | | |
| BCY13279 | BCY13125 | C-term dLys | N-(acid- | BCY8928 | dLys |
| | | | PEG3)-N- | | (PYA)4 |
| | | | bis(PEG3- | | |
| | | | azide) | | |
| BCY13283 | BCY13129 | C-term dLys | N-(acid- | BCY8928 | dLys |
| | | | PEG3)-N- | | (PYA)4 |
| | | | bis(PEG3- | | |
| | | | azide) | | |
| BCY13287 | BCY13133 | N-terminus | N-(acid- | BCY8928 | dLys |
| | | | PEG3)-N- | | (PYA)4 |
| | | | bis(PEG3- | | |
| | | | azide) | | |
| BCY14049 | BCY13917 | N-terminus | N-(acid- | BCY8928 | dLys |
| | | | PEG3)-N- | | (PYA)4 |
| | | | bis(PEG3- | | |
| | | | azide) | | |
| BCY14050 | BCY13918 | N-terminus | N-(acid- | BCY8928 | dLys |
| | | | PEG3)-N- | | (PYA)4 |
| | | | bis(PEG3- | | |
| | | | azide) | | |
| BCY14051 | BCY13919 | N-terminus | N-(acid- | BCY8928 | dLys |
| | | | PEG3)-N- | | (PYA)4 |
| | | | bis(PEG3- | | |
| | | | azide) | | |
| BCY14052 | BCY13920 | N-terminus | N-(acid- | BCY8928 | dLys |
| | | | PEG3)-N- | | (PYA)4 |
| | | | bis(PEG3- | | |
| | | | azide) | | |
| BCY14053 | BCY13922 | N-terminus | N-(acid- | BCY8928 | dLys |
| | | | PEG3)-N- | | (PYA)4 |
| | | | bis(PEG3- | | |
| | | | azide) | | |
| BCY14054 | BCY13923 | N-terminus | N-(acid- | BCY8928 | dLys |
| | | | PEG3)-N- | | (PYA)4 |
| | | | bis(PEG3- | | |
| | | | azide) | | |
| BCY14055 | BCY14047 | N-terminus | N-(acid- | BCY8928 | dLys |
| | | | PEG3)-N- | | (PYA)4 |
| | | | bis(PEG3- | | |
| | | | azide) | | |
| BCY14056 | BCY14048 | N-terminus | N-(acid- | BCY8928 | dLys |
| | | | PEG3)-N- | | (PYA)4 |
| | | | bis(PEG3- | | |
| | | | azide) | | |
| BCY14334 | BCY14313 | N-terminus | N-(acid- | BCY8928 | dLys |
| | | | PEG3)-N- | | (PYA)4 |
| | | | bis(PEG3- | | |
| | | | azide) | | |
| BCY14335 | BCY14327 | Lys 8 | N-(acid- | BCY8928 | dLys |
| | | | PEG3)-N- | | (PYA)4 |
| | | | bis(PEG3- | | |
| | | | azide) | | |
| BCY14413 | BCY9594 | N-terminus | N-(acid- | BCY8928 | dLys |
| | | | PEG3)-N- | BCY13389 | (PYA)4 |
| | | | bis(PEG3- | | dLys |
| | | | azide) | | (PYA)4 |
| BCY14414 | BCY13118 | N-terminus | N-(acid- | BCY8928 | dLys |
| | | | PEG3)-N- | BCY13389 | (PYA)4 |
| | | | bis(PEG3- | | dLys |
| | | | azide) | | (PYA)4 |
| BCY15217 | BCY13118 | N-terminus | N-(acid- | BCY14601 | dLys |
| | | | PEG3)-N- | BCY14601 | (PYA)4 |
| | | | bis(PEG3- | | dLys |
| | | | azide) | | (PYA)4 |
| BCY15218 | BCY13118 | N-terminus | N-(acid- | BCY8928 | dLys |
| | | | PEG3)-N- | BCY14601 | (PYA)4 |
| | | | bis(PEG3- | | dLys |
| | | | azide) | | (PYA)4 |

In some embodiments, the heterotandem bicyclic peptide complex is selected from: BCY12491, BCY12730, BCY13048, BCY13050, BCY13053 and BCY13272.

In some embodiments, the heterotandem bicyclic peptide complex is selected from: BCY12491, BCY12730, BCY13048, BCY13050 and BCY13053.

In some embodiments, the heterotandem bicyclic peptide complex is BCY12491.

The heterotandem bicyclic peptide complex BCY12491 consists of a EphA2 specific peptide BCY9594 linked to two CD137 specific peptides (both of which are BCY8928) via a N-(acid-PEG₃)-N-bis(PEG₃-azide) linker, shown pictorially as:

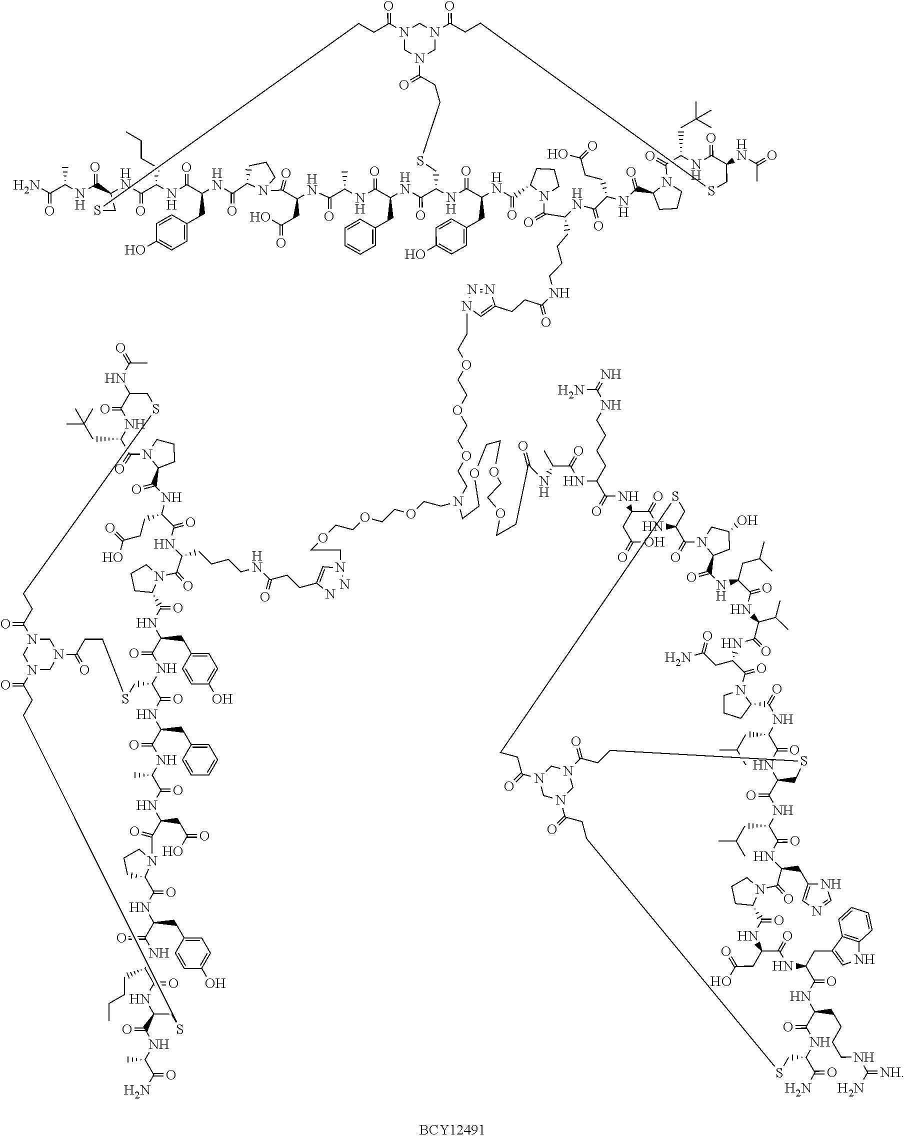

It has been found that BCY12491 leads to a significant anti-tumor response and modulation (increase) of the tumor infiltrating immune cells and immune response.

In some embodiments, the heterotandem bicyclic peptide complex is BCY13272.

The heterotandem bicyclic peptide complex BCY13272 consists of a EphA2 specific peptide BCY13118 linked to two CD137 specific peptides (both of which are BCY8928) via a N-(acid-PEG₃)-N-bis(PEG₃-azide) linker, shown pictorially as:

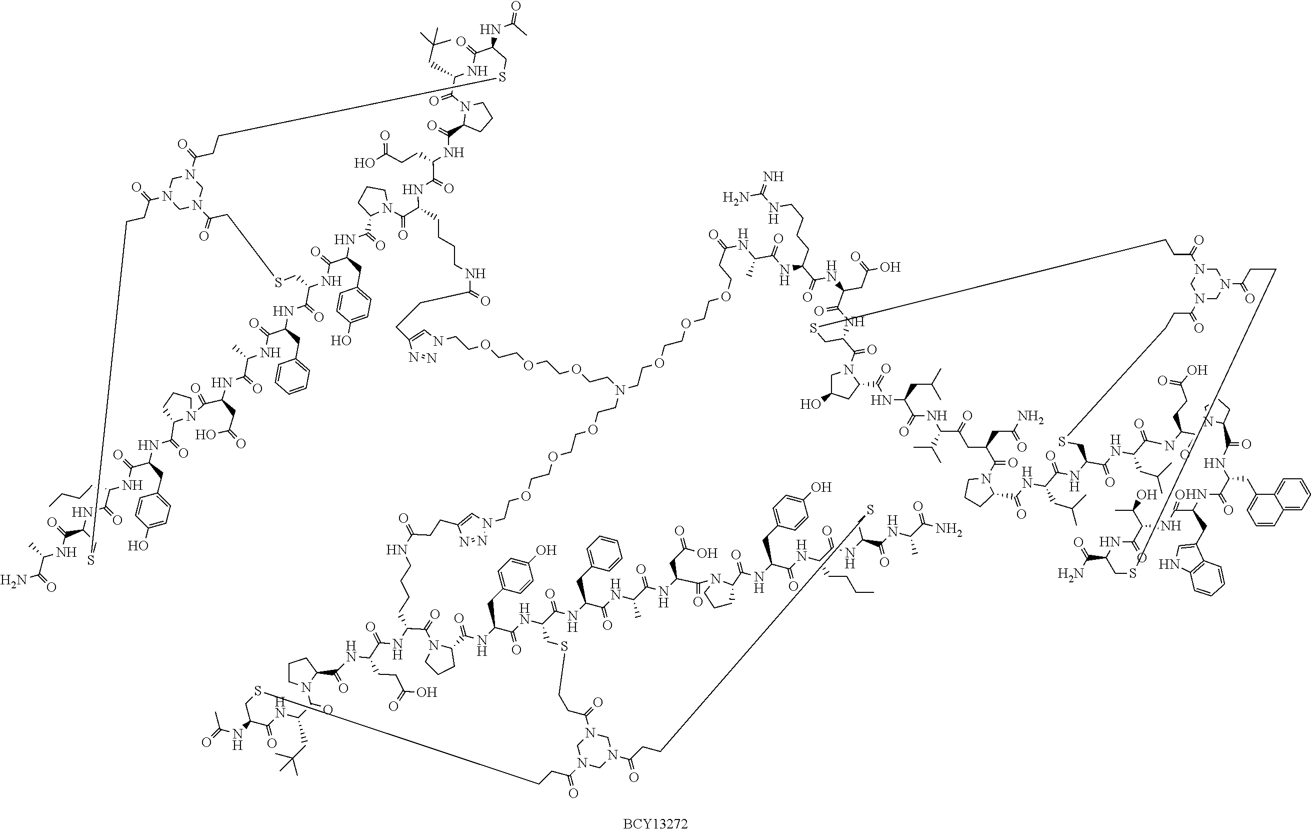

It has been found that BCY13272 leads to a significant antitumor effect in a MC38 tumor model in mice.

In some embodiments, where a heterotandem bicyclic peptide complex comprises two or more CD137 binding peptide ligands, the first peptide ligand comprises a PD-Li binding bicyclic peptide ligand attached to a TATA scaffold, each of the two or more CD137 binding bicyclic peptide ligands attached to a TATA scaffold, and said heterotandem bicyclic peptide complex is selected from the complexes listed in Table D:

TABLE D
(PD-L1:CD137; 1:2)

| Complex | | Attachment | | | Attachment |
|---|---|---|---|---|---|
| No. | PD-L1 BCY No. | Point | Linker | CD137 BCY No. | Point |
| BCY11780 | BCY10861 | Lys(PYA)9 | TCA-[Peg₁₀]₃ | BCY8928 | dLys4 |
| BCY12662 | BCY12479 | C-term Lys | N-(acid-PEG₃)- | BCY8928 | dLys(PYA)4 |
| | | | N-bis(PEG₃-azide) | | |
| BCY12722 | BCY12477 | C-term Lys | N-(acid-PEG₃)- | BCY8928 | dLys(PYA)4 |
| | | | N-bis(PEG₃-azide) | | |

In some embodiments, where a heterotandem bicyclic peptide complex comprises one CD137 binding peptide ligands, the first peptide ligand comprises a PD-Li binding bicyclic peptide ligand attached to a TATA scaffold, the one CD137 binding peptide ligand is attached to a TATA scaffold, and said heterotandem bicyclic peptide complex is selected from the complexes listed in Table E:

TABLE E
(PD-L1:CD137; 1:1)

| Complex | PD-L1 | Attachment | | CD137 | Attachment |
|---|---|---|---|---|---|
| No. | BCY No. | Point | Linker | BCY No. | Point |
| BCY12229 | BCY11865 | Lys9 | Peg5 | BCY8928 | dLys(PYA)4 |
| BCY12230 | BCY11866 | Lys2 | Peg5 | BCY8928 | dLys(PYA)4 |
| BCY12231 | BCY11867 | Lys7 | Peg5 | BCY8928 | dLys(PYA)4 |
| BCY12232 | BCY11868 | Lys8 | Peg5 | BCY8928 | dLys(PYA)4 |
| BCY12242 | BCY11869 | Lys11 | Peg5 | BCY8928 | dLys(PYA)4 |
| BCY12375 | BCY10861 | Lys(PYA)9 | Peg5 | BCY12023 | dLys4 |
| BCY12663 | BCY12479 | C-term Lys | Peg5 | BCY8928 | dLys(PYA)4 |
| BCY12796 | BCY12477 | C-term Lys | Peg5 | BCY8928 | dLys(PYA)4 |
| BCY12021 | BCY10861 | Lys(PYA)9 | Peg5 | BCY11144 | dLys4 |

In some embodiments, a heterotandem bicyclic peptide complex is selected from: BCY12375 and BCY12021.

In some embodiments, where a heterotandem bicyclic peptide complex comprises one CD137 binding peptide ligand, the first peptide ligand comprises a PD-L1 binding bicyclic peptide ligand attached to a TATA scaffold, the one CD137 binding peptide ligand is attached to a TATA scaffold, and said heterotandem bicyclic peptide complex is selected from the complexes listed in Table E-2:

TABLE E-2
(PD-L1:CD137; 1:1)

| Complex | PD-L1 | Attachment | | CD137 | Attachment |
|---|---|---|---|---|---|
| No. | BCY No. | Point | Linker | BCY No. | Point |
| BCY8939 | BCY8938 | N-terminal | —PEG₁₂— | BCY7732 | C-terminal |
| | | PYA | | | Dap |
| BCY10580 | BCY10043 | N-terminal | —PEG₁₂— | BCY9172 | C-terminal |
| | | PYA | | | Dap |
| BCY10581 | BCY10044 | C-terminal | —PEG₁₂— | BCY9172 | C-terminal |
| | | Lys(PYA) | | | Dap |
| BCY10582 | BCY10045 | Lys(PYA)9 | —PEG₁₂— | BCY9172 | C-terminal |
| | | | | | Dap |
| BCY11017 | BCY10861 | Lys(PYA)9 | —PEG₁₂— | BCY8919 | Lys3 |
| BCY11018 | BCY10861 | Lys(PYA)9 | —PEG₁₂— | BCY8920 | dLys4 |
| BCY11019 | BCY10861 | Lys(PYA)9 | —PEG₁₂— | BCY9172 | C-terminal |
| | | | | | Dap |
| BCY11376 | BCY10861 | Lys(PYA)9 | —CH₂— | BCY8919 | Lys3 |
| BCY11377 | BCY10861 | Lys(PYA)9 | —CH₂— | BCY8920 | dLys4 |
| BCY11378 | BCY10861 | Lys(PYA)9 | —CH₂— | BCY9172 | C-terminal |
| | | | | | Dap |
| BCY11379 | BCY10861 | Lys(PYA)9 | —PEG₅— | BCY8919 | Lys3 |
| BCY11380 | BCY10861 | Lys(PYA)9 | —PEG₅— | BCY8920 | dLys4 |
| BCY11381 | BCY10861 | Lys(PYA)9 | —PEG₅— | BCY9172 | C-terminal |
| | | | | | Dap |

In some embodiments, where a heterotandem bicyclic peptide complex comprises one CD137 binding peptide ligands, the first peptide ligand comprises an EphA2 binding bicyclic peptide ligand attached to a TATA scaffold, the one CD137 binding peptide ligand is attached to a TATA scaffold, and said heterotandem complex is selected from the complexes listed in Table F:

TABLE F
(EphA2:CD137; 1:1)

| Complex | EphA2 | Attachment | Link- | CD137 | Attachment |
|---|---|---|---|---|---|
| No. | BCY No. | Point | er | BCY No. | Point |
| BCY12233 | BCY11813 | N-term | Peg5 | BCY8920 | dLys4 |
| | | PYA | | | |
| BCY12234 | BCY11814 | C-term | Peg5 | BCY8920 | dLys4 |
| | | Lys(PYA) | | | |
| BCY12235 | BCY11815 | Lys(PYA) | Peg5 | BCY8920 | dLys4 |
| | | 8 | | | |
| BCY12236 | BCY11816 | Lys(PYA)2 | Peg5 | BCY8920 | dLys4 |
| BCY12237 | BCY11817 | Lys(PYA)7 | Peg5 | BCY8920 | dLys4 |
| BCY12711 | BCY9594 | N-terminus | Peg5 | BCY12143 | dLys (PYA)4 |
| BCY12712 | BCY9594 | N-terminus | Peg5 | BCY12149 | dLys (PYA)4 |
| BCY12713 | BCY9594 | N-terminus | Peg5 | BCY12147 | dLys (PYA)4 |
| BCY12714 | BCY9594 | N-terminus | Peg5 | BCY12145 | dLys (PYA)4 |
| BCY12715 | BCY9594 | N-terminus | Peg5 | BCY12146 | dLys (PYA)4 |
| BCY12717 | BCY9594 | N-terminus | Peg5 | BCY12352 | dLys (PYA)4 |
| BCY12718 | BCY9594 | N-terminus | Peg5 | BCY12353 | dLys (PYA)4 |
| BCY12719 | BCY9594 | N-terminus | Peg5 | BCY12354 | dLys (PYA)4 |
| BCY12720 | BCY9594 | N-terminus | Peg5 | BCY12360 | dLys (PYA)4 |
| BCY12961 | BCY12734 | C-term Lys | Peg5 | BCY8928 | dLys (PYA)4 |
| BCY12962 | BCY12735 | Lys8 | Peg5 | BCY8928 | dLys (PYA)4 |
| BCY12963 | BCY12736 | Lys2 | Peg5 | BCY8928 | dLys (PYA)4 |
| BCY12964 | BCY12737 | Lys7 | Peg5 | BCY8928 | dLys (PYA)4 |
| BCY12965 | BCY12738 | N-terminus | Peg5 | BCY8928 | dLys (PYA)4 |
| BCY12966 | BCY12739 | N-terminus | Peg5 | BCY8928 | dLys (PYA)4 |
| BCY13029 | BCY12854 | N-terminus | Peg5 | BCY8928 | dLys (PYA)4 |
| BCY13030 | BCY12855 | N-terminus | Peg5 | BCY8928 | dLys (PYA)4 |
| BCY13031 | BCY12856 | N-terminus | Peg5 | BCY8928 | dLys (PYA)4 |
| BCY13032 | BCY12857 | N-terminus | Peg5 | BCY8928 | dLys (PYA)4 |
| BCY13033 | BCY12858 | N-terminus | Peg5 | BCY8928 | dLys (PYA)4 |
| BCY13034 | BCY12859 | N-terminus | Peg5 | BCY8928 | dLys (PYA)4 |
| BCY13035 | BCY12860 | N-terminus | Peg5 | BCY8928 | dLys (PYA)4 |
| BCY13036 | BCY12861 | N-terminus | Peg5 | BCY8928 | dLys (PYA)4 |
| BCY13037 | BCY12862 | N-terminus | Peg5 | BCY8928 | dLys (PYA)4 |
| BCY13038 | BCY12863 | N-terminus | Peg5 | BCY8928 | dLys (PYA)4 |
| BCY13039 | BCY12864 | N-terminus | Peg5 | BCY8928 | dLys (PYA)4 |
| BCY13040 | BCY12865 | N-terminus | Peg5 | BCY8928 | dLys (PYA)4 |
| BCY13041 | BCY12866 | N-terminus | Peg5 | BCY8928 | dLys (PYA)4 |
| BCY13141 | BCY12856 | N-terminus | Peg5 | BCY12353 | dLys (PYA)4 |
| BCY13142 | BCY9594 | N-terminus | Peg5 | BCY13137 | dLys (PYA)4 |
| BCY13143 | BCY12856 | N-terminus | Peg5 | BCY13137 | dLys (PYA)4 |
| BCY13250 | BCY13116 | N-terminus | Peg5 | BCY8928 | dLys (PYA)4 |
| BCY13251 | BCY13117 | N-terminus | Peg5 | BCY8928 | dLys (PYA)4 |
| BCY13252 | BCY13118 | N-terminus | Peg5 | BCY8928 | dLys (PYA)4 |
| BCY13253 | BCY13119 | N-terminus | Peg5 | BCY8928 | dLys (PYA)4 |
| BCY13254 | BCY13120 | C-term | Peg5 | BCY8928 | dLys (PYA)4 |
| | | dLys | | | |
| BCY13255 | BCY13121 | N-terminus | Peg5 | BCY8928 | dLys (PYA)4 |
| BCY13256 | BCY13122 | N-terminus | Peg5 | BCY8928 | dLys (PYA)4 |
| BCY13257 | BCY13123 | N-terminus | Peg5 | BCY8928 | dLys (PYA)4 |
| BCY13258 | BCY13124 | N-terminus | Peg5 | BCY8928 | dLys (PYA)4 |
| BCY13260 | BCY13126 | N-terminus | Peg5 | BCY8928 | dLys (PYA)4 |
| BCY13261 | BCY13127 | N-terminus | Peg5 | BCY8928 | dLys (PYA)4 |
| BCY13262 | BCY13128 | N-terminus | Peg5 | BCY8928 | dLys (PYA)4 |
| BCY13264 | BCY13130 | N-terminus | Peg5 | BCY8928 | dLys (PYA)4 |
| BCY13265 | BCY13131 | N-terminus | Peg5 | BCY8928 | dLys (PYA)4 |
| BCY13266 | BCY13132 | N-terminus | Peg5 | BCY8928 | dLys (PYA)4 |
| BCY13268 | BCY13134 | N-terminus | Peg5 | BCY8928 | dLys (PYA)4 |
| BCY13269 | BCY13135 | N-terminus | Peg5 | BCY8928 | dLys (PYA)4 |
| BCY13340 | BCY12865 | N-terminus | Peg5 | BCY12353 | dLys (PYA)4 |
| BCY13342 | BCY12860 | N-terminus | Peg5 | BCY12353 | dLys (PYA)4 |

In some embodiments, a heterotandem bicyclic peptide complex is selected from: BCY13035, BCY13040, BCY13253, BCY13254, BCY13340 and BCY13342.

In some embodiments, where a heterotandem bicyclic peptide complex comprises one CD137 binding peptide ligand, the first peptide ligand comprises an EphA2 binding bicyclic peptide ligand attached to a TATA scaffold, the one CD137 binding peptide ligand is attached to a TATA scaffold, and said heterotandem complex is selected from the complexes listed in Table F-2:

TABLE F-2
(EphA2:CD137; 1:1)

| Complex | EphA2 | Attachment | | CD137 | Attachment |
|---|---|---|---|---|---|
| No. | BCY No. | Point | Linker | BCY No. | Point |
| BCY9173 | BCY6169 | N-terminal | —PEG₁₂— | BCY9172 | C-terminal |
| | | PYA | | | Dap |
| BCY7985 | BCY6169 | N-terminal | —PEG₁₂— | BCY7732 | C-terminal |
| | | PYA | | | Dap |
| BCY8942 | BCY6169 | N-terminal | —PEG₁₂— | BCY8045 | C-terminal |
| | | PYA | | | Dap |
| BCY8943 | BCY8941 | N-terminal | —PEG₁₂— | BCY7732 | C-terminal |
| | | PYA | | | Dap |
| BCY9647 | BCY6099 | N-terminus | —PEG₁₀— | BCY7741 | C-terminal |
| | | | | | Dap(PYA) |
| BCY9648 | BCY6099 | N-terminus | —PEG₂₃— | BCY7741 | C-terminal |
| | | | | | Dap(PYA) |
| BCY9655 | BCY6099 | N-terminus | —PEG₁₅— | BCY7741 | C-terminal |
| | | | Sar₅— | | Dap(PYA) |
| | | | (SEQ ID | | |
| | | | NO: 276) | | |
| BCY9656 | BCY6099 | N-terminus | —PEG₁₀— | BCY7741 | C-terminal |
| | | | Sar₁₀— | | Dap(PYA) |
| | | | (SEQ ID | | |
| | | | NO: 277) | | |
| BCY9657 | BCY6099 | N-terminus | —PEG₅— | BCY7741 | C-terminal |
| | | | Sar₁₅— | | Dap(PYA) |
| | | | (SEQ ID | | |
| | | | NO: 278) | | |
| BCY9658 | BCY6099 | N-terminus | —PEG₅— | BCY7741 | C-terminal |
| | | | Sar₅— | | Dap(PYA) |
| | | | (SEQ ID | | |
| | | | NO: 279) | | |
| BCY9659 | BCY6099 | N-terminus | —PEG₅— | BCY7741 | C-terminal |
| | | | | | Dap(PYA) |
| BCY9758 | BCY6099 | N-terminus | —PEG₂₄— | BCY7732 | C-terminal |
| | | | | | Dap |
| BCY10568 | BCY6169 | N-terminal | —PEG₁₂— | BCY8919 | Lys3 |
| | | PYA | | | |
| BCY10570 | BCY6169 | N-terminal | —PEG₁₂— | BCY8920 | dLys4 |
| | | PYA | | | |
| BCY10574 | BCY9594 | N-terminus | —PEG₅— | BCY8927 | Lys (PYA)3 |
| BCY10575 | BCY9594 | N-terminus | —PEG₅— | BCY8928 | dLys |
| | | | | | (PYA)4 |
| BCY10576 | BCY9594 | N-terminus | —PEG₅— | BCY11014 | C-terminal |
| | | | | | Dap(PYA) |
| BCY10577 | BCY6169 | N-terminus | —CH₂— | BCY9172 | C-terminal |
| | | | | | Dap |

In some embodiments, a heterotandem bicyclic peptide complex is BCY7985, wherein a CD137-specific peptide BCY7859 linked to the N-terminal PYA group of an EphA2-specific peptide BCY6169 via PEG₁₂:

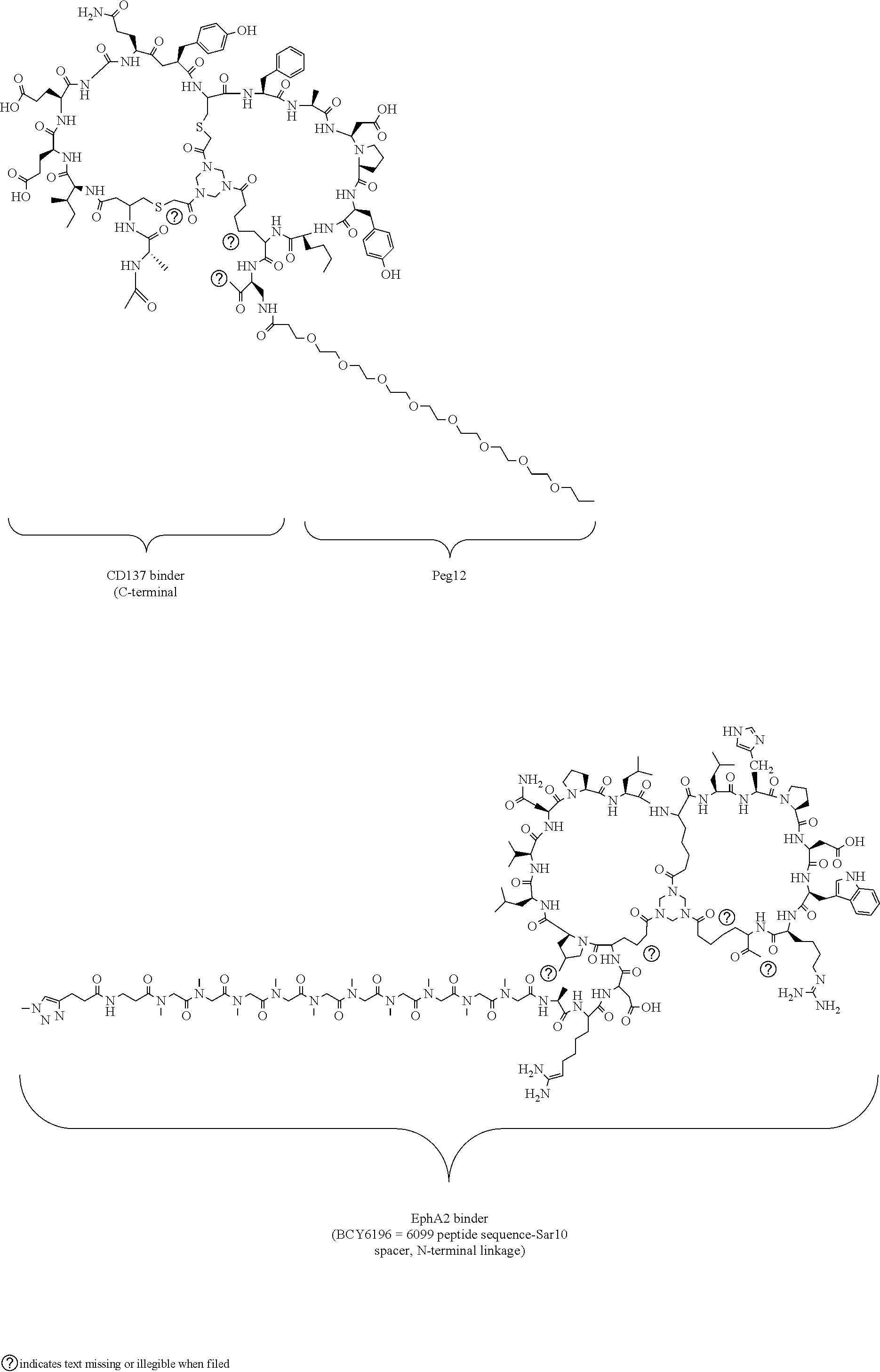

In some embodiments, where a heterotandem bicyclic peptide complex comprises one CD137 binding peptide ligands, the first peptide ligand comprises a Nectin-4 binding bicyclic peptide ligand attached to a TATA scaffold, the one CD137 binding peptide ligand is attached to a TATA scaffold, and said heterotandem complex is selected from the complexes listed in Table G:

TABLE G
(Nectin-4:CD137; 1:1)

| Complex | Nectin-4 | Attachment | | CD137 | Attachment |
|---|---|---|---|---|---|
| No. | BCY No. | Point | Linker | BCY No | Point |
| BCY11616 | BCY8116 | N-terminus | Peg5 | BCY7744 | dLys(PYA)4 |
| BCY12238 | BCY12024 | dLys3 | Peg5 | BCY8928 | dLys(PYA)4 |
| BCY12377 | BCY8116 | N-terminus | Peg5 | BCY12143 | dLys(PYA)4 |
| BCY12379 | BCY8116 | N-terminus | Peg5 | BCY12149 | dLys(PYA)4 |
| BCY12572 | BCY8116 | N-terminus | Peg5 | BCY12352 | dLys(PYA)4 |
| BCY12573 | BCY8116 | N-terminus | Peg5 | BCY12353 | dLys(PYA)4 |
| BCY12574 | BCY8116 | N-terminus | Peg5 | BCY12354 | dLys(PYA)4 |
| BCY12575 | BCY8116 | N-terminus | Peg5 | BCY12360 | dLys(PYA)4 |
| BCY12576 | BCY12363 | dLys3 | Peg5 | BCY8928 | dLys(PYA)4 |
| BCY12577 | BCY12364 | dLys3 | Peg5 | BCY8928 | dLys(PYA)4 |
| BCY12578 | BCY12365 | dLys3 | Peg5 | BCY8928 | dLys(PYA)4 |
| BCY12579 | BCY12366 | dLys3 | Peg5 | BCY8928 | dLys(PYA)4 |
| BCY12580 | BCY12367 | dLys3 | Peg5 | BCY8928 | dLys(PYA)4 |
| BCY12581 | BCY12368 | N-terminus | Peg5 | BCY8928 | dLys(PYA)4 |
| BCY12582 | BCY12369 | N-terminus | Peg5 | BCY8928 | dLys(PYA)4 |
| BCY12583 | BCY12370 | N-terminus | Peg5 | BCY8928 | dLys(PYA)4 |
| BCY12584 | BCY12371 | dLys3 | Peg5 | BCY8928 | dLys(PYA)4 |
| BCY12585 | BCY12384 | N-terminus | Peg5 | BCY8928 | dLys(PYA)4 |
| BCY12709 | BCY8116 | N-terminus | Peg5 | BCY12381 | dLys(PYA)4 |
| BCY12710 | BCY8116 | N-terminus | Peg5 | BCY12382 | dLys(PYA)4 |
| BCY11468 | BCY11016 | N-term | TCA- | BCY8928 | dLys(PYA)4 |
| | | PYA | [Peg10]3 | | |
| BCY11618 | BCY11143 | N-term | Peg5 | BCY8920 | dLys4 |
| | | PYA | | | |
| BCY11776 | BCY8116 | N-terminus | Peg5 | BCY11144 | C-term |
| | | | | | Dap(PYA) |
| BCY11860 | BCY11143 | N-term | Peg5 | BCY8920 | dLys4 |
| | | PYA | | | |
| BCY12020 | BCY11016 | N-term | Peg5 | BCY11144 | C-term |
| | | PYA | | | Dap(PYA) |
| BCY12661 | BCY11015 | N-term | Peg5 | BCY12023 | dLys4 |
| | | PYA | | | |
| BCY12969 | BCY8116 | N-terminus | Peg5 | BCY12358 | dLys(PYA)4 |

In some embodiments, a heterotandem bicyclic peptide complex is selected from: BCY11468, BCY11618, BCY11776, BCY11860, BCY122, BCY12661 and BCY12969.

In some embodiments, where a heterotandem bicyclic peptide complex comprises one CD137 binding peptide ligand, the first peptide ligand comprises a Nectin-4 binding bicyclic peptide ligand attached to a TATA scaffold, the one CD137 binding peptide ligand is attached to a TATA scaffold, and said heterotandem complex is selected from the complexes listed in Table G-2:

TABLE G-2
(Nectin-4:CD137; 1:1)

| Complex | Nectin-4 | Attachment | | CD137 | Attachment |
|---|---|---|---|---|---|
| No. | BCY No. | Point | Linker | BCY No. | Point |
| BCY8854 | BCY8846 | N-terminal | —PEG₁₂- | BCY7732 | C-terminal Dap |
| | | PYA | | | |
| BCY9350 | BCY11942 | N-terminal | —PEG₁₂- | BCY7732 | C-terminal Dap |
| | | PYA | | | |
| BCY9351 | BCY8846 | N-terminal | —PEG₁₂- | BCY8045 | C-terminal Dap |
| | | PYA | | | |
| BCY9399 | BCY8116 | N-terminus | —PEG₁₀- | BCY7741 | C-terminal Dap(PYA) |
| BCY9400 | BCY8116 | N-terminus | —PEG₂₃- | BCY7741 | C-terminal Dap(PYA) |
| BCY9401 | BCY8116 | N-terminus | —B—Ala—Sar₂₀— | BCY7741 | C-terminal Dap(PYA) |
| | | | (SEQ ID NO: | | |
| | | | 280) | | |
| BCY9403 | BCY8116 | N-terminus | —B—Ala—Sar₁₀— | BCY7741 | C-terminal Dap(PYA) |
| | | | PEG₁₀— (SEQ | | |
| | | | ID NO: 281) | | |
| BCY9405 | BCY8116 | N-terminus | —B—Ala—Sars— | BCY7741 | C-terminal Dap(PYA) |
| | | | PEG₁₅— (SEQ | | |
| | | | ID NO: 282) | | |
| BCY9406 | BCY8116 | N-terminus | —B—Ala—Sar₅— | BCY7741 | C-terminal Dap(PYA) |
| | | | PEG₅— (SEQ | | |
| | | | ID NO: 283) | | |
| BCY9407 | BCY8116 | N-terminus | —PEG₁₅—Sar₅— | BCY7741 | C-terminal Dap(PYA) |
| | | | (SEQ ID NO: | | |
| | | | 276) | | |
| BCY9408 | BCY8116 | N-terminus | —PEG₁₀—Sar₁₀— | BCY7741 | C-terminal Dap(PYA) |
| | | | (SEQ ID NO: | | |
| | | | 277) | | |
| BCY9409 | BCY8116 | N-terminus | —PEG₅-Sar₁₅— | BCY7741 | C-terminal Dap(PYA) |
| | | | (SEQ ID NO: | | |
| | | | 278) | | |
| BCY9410 | BCY8116 | N-terminus | —PEG₅—Sar₅— | BCY7741 | C-terminal Dap(PYA) |
| | | | (SEQ ID NO: | | |
| | | | 279) | | |
| BCY9411 | BCY8116 | N-terminus | —PEG₅— | BCY7741 | C-terminal Dap(PYA) |
| BCY9759 | BCY8116 | N-terminus | —PEG₂₄— | BCY7732 | C-terminal Dap |
| BCY10000 | BCY8846 | N-terminal | —PEG₁₂— | BCY9172 | C-terminal Dap |
| | | PYA | | | |
| BCY10567 | BCY8846 | N-terminal | —PEG₁₂— | BCY8919 | Lys3 |
| | | PYA | | | |
| BCY10569 | BCY8846 | N-terminal | —PEG₁₂— | BCY8920 | dLys4 |
| | | PYA | | | |
| BCY10571 | BCY8116 | N-terminus | —PEG₅— | BCY8927 | Lys(PYA)3 |
| BCY10572 | BCY8116 | N-terminus | —PEG₅— | BCY8928 | dLys (PYA)4 |
| BCY10573 | BCY8116 | N-terminus | —PEG₅— | BCY11014 | C-terminal Dap(PYA) |
| BCY10578 | BCY8846 | N-terminal | —CH₂— | BCY9172 | C-terminal Dap |
| | | PYA | | | |
| BCY10917 | BCY8831 | dLys(Sar₁₀)- | —PEG₁₂— | BCY11014 | C-terminal Dap(PYA) |
| | | (B-Ala))4 | | | |
| BCY11020 | BCY8831 | dLys(Sar₁₀)- | —PEG₅— | BCY11014 | C-terminal Dap(PYA) |
| | | (B-Ala))4 | | | |
| BCY11373 | BCY8116 | N-terminus | —CH₂— | BCY8927 | Lys(PYA)3 |
| BCY11374 | BCY8116 | N-terminus | —CH₂— | BCY8928 | dLys (PYA)4 |
| BCY11375 | BCY8116 | N-terminus | —CH₂— | BCY11014 | C-terminal Dap(PYA) |
| BCY11616 | BCY8116 | N-terminus | —PEG₅— | BCY7744 | dLys (PYA)4 |
| BCY11617 | BCY8116 | N-terminus | —PEG₅— | BCY11506 | Lys(PYA)4 |
| BCY11857 | BCY11414 | N-terminus | —PEG₅— | BCY7744 | dLys (PYA)4 |
| BCY11858 | BCY11414 | N-terminus | —PEG₅— | BCY8928 | dLys (PYA)4 |
| BCY11859 | BCY11415 | N-terminus | —PEG₅— | BCY8928 | dLys (PYA)4 |

In some embodiments, a heterotandem bicyclic peptide complex is selected from those disclosed in U.S. patent application Ser. No. 17/062,662, the contents of which are incorporated herein by reference in their entireties.

In some embodiments, a heterotandem bicyclic peptide complex is selected from those disclosed in US Patent Publication 20190307836, the contents of which are incorporated herein by reference in their entireties.

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4^{th} ed., John Wiley & Sons, Inc.), which are incorporated herein by reference.

### Nomenclature

### Numbering

When referring to amino acid residue positions within compounds of the invention, cysteine residues (Cᵢ, Cᵢᵢ and Cᵢᵢᵢ) are omitted from the numbering as they are invariant, therefore, the numbering of amino acid residues within SEQ ID NO: 1 is referred to as below:

(SEQ ID NO: 1)
Cᵢ-P1-1Nal2-dD3-Cᵢ-M4-HArgs-D6-W7-
S8-T9-P10-HyP11-W12-Cᵢᵢᵢ.

For the purpose of this description, all bicyclic peptides are assumed to be cyclised with TBMB (1,3,5-tris(bromomethyl)benzene) or 1,1′,1″-(1,3,5-triazinane-1,3,5-triyl)triprop-2-en-1-one (TATA) and yielding a tri-substituted structure. Cyclisation with TBMB and TATA occurs on Cᵢ, Cᵢᵢ, and Cᵢᵢᵢ.

### Molecular Format

N- or C-terminal extensions to the bicycle core sequence are added to the left or right side of the sequence, separated by a hyphen. For example, an N-terminal βAla-Sar10-Ala tail would be denoted as:

(SEQ ID NO: X)
βAla-Sar10-A-.

In light of the disclosure in Nair et al (2003) J Immunol 170(3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa). For the avoidance of doubt, references to amino acids either as their full name or as their amino acid single or three letter codes are intended to be represented herein as L-amino acids unless otherwise stated. If such an amino acid is intended to be represented as a D-amino acid then the amino acid will be prefaced with a lower case d within square parentheses, for example [dA], [dD], [dE], [dK], [d1Nal], [dNle], etc.

### Advantages of the Peptide Ligands

Certain heterotandem bicyclic peptide complexes of the present invention have a number of advantageous properties which enable them to be considered as suitable drug-like molecules for injection, inhalation, nasal, ocular, oral or topical administration. Such advantageous properties include:
Species cross-reactivity. This is a typical requirement for preclinical pharmacodynamics and pharmacokinetic evaluation;
Protease stability. Heterotandem bicyclic peptide complexes should ideally demonstrate stability to plasma proteases, epithelial (“membrane-anchored”) proteases, gastric and intestinal proteases, lung surface proteases, intracellular proteases and the like. Protease stability should be maintained between different species such that a heterotandem bicyclic peptide lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes;
Selectivity. Certain heterotandem bicyclic peptide complexes of the invention demonstrate good selectivity over other targets;
An optimal plasma half-life in the circulation. Depending upon the clinical indication and treatment regimen, it may be required to develop a heterotandem bicyclic peptide complex for short exposure in an acute illness management setting, or develop a heterotandem bicyclic peptide complex with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent.
Crucially, data is presented herein where selected heterotandem bicyclic peptide complexes demonstrate anti-tumor efficacy when dosed at a frequency that does not maintain plasma concentrations above the in vitro EC₅₀ of the compound. This is in contrast to larger recombinant biologic (i.e. antibody based) approaches to CD137 agonism or bispecific CD137 agonism (Segal et al., Clin Cancer Res., 23(8):1929-1936 (2017), Claus et al., Sci Trans Med., 11(496): eaav5989, 1-12 (2019), Hinner et al., Clin Cancer Res., 25(19):5878-5889 (2019)). Without being bound by theory, the reason for this observation is thought to be due to the fact that heterotandem bicycle complexes have relatively low molecular weight (typically <15 kDa), they are fully synthetic and they are tumor targeted agonists of CD137. As such, they have relatively short plasma half lives but good tumor penetrance and retention. Data is presented herein which fully supports these advantages. For example, anti-tumor efficacy in syngeneic rodent models in mice with humanized CD137 is demonstrated either daily or every 3^{rd} day. In addition, intraperitoneal pharmacokinetic data shows that the plasma half life is <3 hours, which would predict that the circulating concentration of the complex would consistently drop below the in vitro EC₅₀ between doses. Furthermore, tumor pharmacokinetic data shows that levels of heterotandem bicycle complex in tumor tissue may be higher and more sustained as compared to plasma levels.
It will be appreciated that this observation forms an important further aspect of the invention. Thus, according to a further aspect of the invention, there is provided a method of treating cancer which comprises administration of a heterotandem bicyclic peptide complex as defined herein at a dosage frequency which does not sustain plasma concentrations of said complex above the in vitro EC₅₀ of said complex.
Immune Memory. Coupling the cancer cell binding bicyclic peptide ligand with the immune cell binding bicyclic peptide ligand provides the synergistic advantage of immune memory. Data is presented herein which demonstrates that selected heterotandem bicyclic peptide complexes of the invention not only eradicate tumors but upon readministration of the tumorigenic agent, none of the inoculated complete responder mice developed tumors (see FIG. 5). This indicates that treatment with the selected heterotandem bicyclic peptide complexes of the invention has induced immunogenic memory in the complete responder mice. This has a significant clinical advantage in order to prevent recurrence of said tumor once it has been initially controlled and eradicated.

### Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three reactive groups selected from cysteine, 3-mercaptopropionic acid and/or cysteamine and form at least two loops on the scaffold.

### Reactive Groups

The molecular scaffold of the invention may be bonded to the polypeptide via functional or reactive groups on the polypeptide. These are typically formed from the side chains of particular amino acids found in the polypeptide polymer. Such reactive groups may be a cysteine side chain, a lysine side chain, or an N-terminal amine group or any other suitable reactive group, such as penicillamine. Details of suitable reactive groups may be found in WO 2009/098450.

Examples of reactive groups of natural amino acids are the thiol group of cysteine, the amino group of lysine, the carboxyl group of aspartate or glutamate, the guanidinium group of arginine, the phenolic group of tyrosine or the hydroxyl group of serine. Non-natural amino acids can provide a wide range of reactive groups including an azide, a keto-carbonyl, an alkyne, a vinyl, or an aryl halide group. The amino and carboxyl group of the termini of the polypeptide can also serve as reactive groups to form covalent bonds to a molecular scaffold/molecular core.

The polypeptides of the invention contain at least three reactive groups. Said polypeptides can also contain four or more reactive groups. The more reactive groups are used, the more loops can be formed in the molecular scaffold.

In a preferred embodiment, polypeptides with three reactive groups are generated. Reaction of said polypeptides with a molecular scaffold/molecular core having a three-fold rotational symmetry generates a single product isomer. The generation of a single product isomer is favourable for several reasons. The nucleic acids of the compound libraries encode only the primary sequences of the polypeptide but not the isomeric state of the molecules that are formed upon reaction of the polypeptide with the molecular core. If only one product isomer can be formed, the assignment of the nucleic acid to the product isomer is clearly defined. If multiple product isomers are formed, the nucleic acid cannot give information about the nature of the product isomer that was isolated in a screening or selection process. The formation of a single product isomer is also advantageous if a specific member of a library of the invention is synthesized. In this case, the chemical reaction of the polypeptide with the molecular scaffold yields a single product isomer rather than a mixture of isomers.

In another embodiment, polypeptides with four reactive groups are generated. Reaction of said polypeptides with a molecular scaffold/molecular core having a tetrahedral symmetry generates two product isomers. Even though the two different product isomers are encoded by one and the same nucleic acid, the isomeric nature of the isolated isomer can be determined by chemically synthesizing both isomers, separating the two isomers and testing both isomers for binding to a target ligand.

In one embodiment of the invention, at least one of the reactive groups of the polypeptides is orthogonal to the remaining reactive groups. The use of orthogonal reactive groups allows the directing of said orthogonal reactive groups to specific sites of the molecular core. Linking strategies involving orthogonal reactive groups may be used to limit the number of product isomers formed. In other words, by choosing distinct or different reactive groups for one or more of the at least three bonds to those chosen for the remainder of the at least three bonds, a particular order of bonding or directing of specific reactive groups of the polypeptide to specific positions on the molecular scaffold may be usefully achieved.

In another embodiment, the reactive groups of the polypeptide of the invention are reacted with molecular linkers wherein said linkers are capable to react with a molecular scaffold so that the linker will intervene between the molecular scaffold and the polypeptide in the final bonded state.

In some embodiments, amino acids of the members of the libraries or sets of polypeptides can be replaced by any natural or non-natural amino acid. Excluded from these exchangeable amino acids are the ones harbouring functional groups for cross-linking the polypeptides to a molecular core, such that the loop sequences alone are exchangeable. The exchangeable polypeptide sequences have either random sequences, constant sequences or sequences with random and constant amino acids. The amino acids with reactive groups are either located in defined positions within the polypeptide, since the position of these amino acids determines loop size.

In one embodiment, a polypeptide with three reactive groups has the sequence (X)ₗY(X)ₘY(X)ₙY(X)ₒ, wherein Y represents an amino acid with a reactive group, X represents a random amino acid, m and n are numbers between 3 and 6 defining the length of intervening polypeptide segments, which may be the same or different, and l and o are numbers between 0 and 20 defining the length of flanking polypeptide segments.

Alternatives to thiol-mediated conjugations can be used to attach the molecular scaffold to the peptide via covalent interactions. Alternatively these techniques may be used in modification or attachment of further moieties (such as small molecules of interest which are distinct from the molecular scaffold) to the polypeptide after they have been selected or isolated according to the present invention—in this embodiment then clearly the attachment need not be covalent and may embrace non-covalent attachment. These methods may be used instead of (or in combination with) the thiol mediated methods by producing phage that display proteins and peptides bearing unnatural amino acids with the requisite chemical reactive groups, in combination small molecules that bear the complementary reactive group, or by incorporating the unnatural amino acids into a chemically or recombinantly synthesised polypeptide when the molecule is being made after the selection/isolation phase. Further details can be found in WO 2009/098450 or Heinis et al., Nat Chem Biol 2009, 5 (7), 502-7.

In some embodiments, the reactive groups are selected from cysteine, 3-mercaptopropionic acid and/or cysteamine residues.

### Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the compounds of the invention contain an amine function, these may form quaternary ammonium salts, for example by reaction with an alkylating agent according to methods well known to the skilled person. Such quaternary ammonium compounds are within the scope of the invention.

### Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In some embodiments, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In some embodiments, the modified derivative comprises an N-terminal modification. In a further embodiment, the N-terminal modification comprises an N-terminal acetyl group. In this embodiment, the N-terminal cysteine group (the group referred to herein as C) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In some embodiments, the N-terminal modification comprises the addition of a molecular spacer group which facilitates the conjugation of effector groups and retention of potency of the bicyclic peptide to its target.

In some embodiments, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In some embodiments, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, Ca-disubstituted derivatives (for example, aminoisobutyric acid, Aib), and cyclo amino acids, a simple derivative being amino-cyclopropylcarboxylic acid.

In some embodiments, the modified derivative comprises the addition of a spacer group. In some embodiments, the modified derivative comprises the addition of a spacer group to the N-terminal cysteine (Cᵢ) and/or the C-terminal cysteine (Cᵢᵢᵢ).

In some embodiments, the modified derivative comprises replacement of one or more oxidation sensitive amino acid residues with one or more oxidation resistant amino acid residues. In some embodiments, the modified derivative comprises replacement of a tryptophan residue with a naphthylalanine or alanine residue. This embodiment provides the advantage of improving the pharmaceutical stability profile of the resultant bicyclic peptide ligand.

In some embodiments, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In some embodiments, the modified derivative comprises replacement of one or more L-amino acid residues with one or more D-amino acid residues. This embodiment is believed to increase proteolytic stability by steric hindrance and by a propensity of D-amino acids to stabilise β-turn conformations (Tugyi et al (2005) PNAS, 102(2), 413-418).

In D-amino acids to stabilise-turn conformations, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons. (for reviews see Gentilucci et al, Curr. Pharmaceutical Design, (2010), 16, 3185-203, and Nestor et al, Curr. Medicinal Chem (2009), 16, 4399-418).

Examples of modified heterotandem bicyclic peptide complexes of the invention include those listed in Tables H and I below:

TABLE H
(EphA2:CD137; 1:2)

| Complex | EphA2 | Attachme | | CD137 | Attachment | |
|---|---|---|---|---|---|---|
| No. | BCY No. | nt Point | Linker | BCY No. | Point | Modifier |
| BCY14415 | BCY9594 | N-terminus | N-(acid- | BCY8928 | dLys (PYA)4 | Peg12-Biotin |
| | | | PEG3)-N- | BCY13389 | dLys (PYA)4 | |
| | | | bis(PEG3-azide) | | | |
| BCY14416 | BCY9594 | N-terminus | N-(acid- | BCY8928 | dLys (PYA)4 | Alexa |
| | | | PEG3)-N- | BCY13389 | dLys (PYA)4 | Fluor ® |
| | | | bis(PEG3-azide) | | | 488 |
| BCY14417 | BCY13118 | N-terminus | N-(acid- | BCY8928 | dLys(PYA)4 | Peg12- |
| | | | PEG3)-N- | BCY13389 | dLys(PYA)4 | Biotin |
| | | | bis(PEG3-azide) | | | |
| BCY14418 | BCY13118 | N-terminus | N-(acid- | BCY8928 | dLys(PYA)4 | Alexa |
| | | | PEG3)-N- | BCY13389 | dLys(PYA)4 | Fluor ® 488 |
| | | | bis(PEG3-azide) | | | |

TABLE I
(Nectin-4:CD137; 1:2)

| Complex | Nectin-4 | Attachment | | CD137 | Attachment | |
|---|---|---|---|---|---|---|
| No. | BCY No. | Point | Linker | BCY No. | Point | Modifier |
| BCY13582 | BCY8116 | N-terminus | N-(acid-PEG₃)-N- | BCY8928, | dLys(PYA)4 | Biotin- |
| | | | bis(PEG₃-azide) | BCY13389 | dLys(PYA)4 | Peg12 |
| BCY13583 | BCY8116 | N-terminus | N-(acid-PEG₃)-N- | BCY8928, | dLys(PYA)4 | Alexa |
| | | | bis(PEG₃-azide) | BCY13389 | dLys(PYA)4 | Fluor 488 |
| BCY13628 | BCY8116 | N-terminus | N-(acid-PEG₃)-N- | BCY8928, | dLys(PYA)4 | Cyanine 5 |
| | | | bis(PEG₃-azide) | BCY13389 | dLys(PYA)4 | |

### Isotopic Variations

The present invention includes all pharmaceutically acceptable (radio)isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies, and to clinically assess the presence and/or absence of the Nectin-4 target on diseased tissues. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

### Molecular Scaffold

Molecular scaffolds are described in, for example, WO 2009/098450 and references cited therein, particularly WO 2004/077062 and WO 2006/078161.

As noted in the foregoing documents, the molecular scaffold may be a small molecule, such as a small organic molecule.

In one embodiment, the molecular scaffold may be a macromolecule. In one embodiment, the molecular scaffold is a macromolecule composed of amino acids, nucleotides or carbohydrates.

In one embodiment, the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

In one embodiment, the molecular scaffold may comprise or may consist of hexahydro-1,3,5-triazine, especially 1,3,5-Triacryloylhexahydro-1,3,5-triazine (‘TATA’), or a derivative thereof.

The molecular scaffold of the invention contains chemical groups that allow functional groups of the polypeptide of the encoded library of the invention to form covalent links with the molecular scaffold. Said chemical groups are selected from a wide range of functionalities including amines, thiols, alcohols, ketones, aldehydes, nitriles, carboxylic acids, esters, alkenes, alkynes, anhydrides, succinimides, maleimides, azides, alkyl halides and acyl halides.

Scaffold reactive groups that could be used on the molecular scaffold to react with thiol groups of cysteines are alkyl halides (or also named halogenoalkanes or haloalkanes).

Examples include bromomethylbenzene (the scaffold reactive group exemplified by TBMB) or iodoacetamide. Other scaffold reactive groups that are used to selectively couple compounds to cysteines in proteins are maleimides, α-unsaturated carbonyl containing compounds and α-halomethylcarbonyl containing compounds. Examples of maleimides which may be used as molecular scaffolds in the invention include: tris-(2-maleimidoethyl)amine, tris-(2-maleimidoethyl)benzene, tris-(maleimido)benzene. An example of an ab unsaturated carbonyl containing compound is 1,1′,1″-(1,3,5-triazinane-1,3,5-triyl)triprop-2-en-1-one (TATA) (Angewandte Chemie, International Edition (2014), 53(6), 1602-1606). An example of an α-halomethylcarbonyl containing compound is N,N′,N″-(benzene-1,3,5-triyl)tris(2-bromoacetamide). Selenocysteine is also a natural amino acid which has a similar reactivity to cysteine and can be used for the same reactions. Thus, wherever cysteine is mentioned, it is typically acceptable to substitute selenocysteine unless the context suggests otherwise.

### Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al (supra).

Thus, the invention also relates to manufacture of polypeptides or conjugates selected as set out herein, wherein the manufacture comprises optional further steps as explained below. In one embodiment, these steps are carried out on the end product polypeptide/conjugate made by chemical synthesis.

Optionally amino acid residues in the polypeptide of interest may be substituted when manufacturing a conjugate or complex.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al. Proc Natl Acad Sci USA. 1994 Dec. 20; 91(26):12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptides to dissociate from each other once within the reducing environment of the cell. In this case, the molecular scaffold (e.g. TATA) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptides, forming a disulfide-linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

### 2. Compounds and Definitions

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N⁺(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Unless otherwise stated, structures depicted herein are also meant to include all isomeric (e.g., enantiomeric, diastereomeric, and geometric (or conformational)) forms of the structure; for example, the R and S configurations for each asymmetric center, Z and E double bond isomers, and Z and E conformational isomers. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention. Additionally, unless otherwise stated, structures depicted herein are also meant to include compounds that differ only in the presence of one or more isotopically enriched atoms. For example, compounds having the present structures including the replacement of hydrogen by deuterium or tritium, or the replacement of a carbon by a ³C- or ¹⁴C-enriched carbon are within the scope of this invention. Such compounds are useful, for example, as analytical tools, as probes in biological assays, or as therapeutic agents in accordance with the present invention.

As used herein, the term “about” refers to within 20% of a given value. In some embodiments, the term “about” refers to within 20%, 19%, 18%, 17%, 16%, 15%, 14%, 13%, 12%, 11%, 10%, 9%, 8%, 7%, 6%, 5%, 4%, 3% 2%, or 1% of a given value.

As used herein, the term “mg/kg” refers to the milligram of medication per kilogram of the body weight of the subject taking the medication.

### 3. Pharmaceutically Acceptable Compositions

According to some embodiments, the present invention provides a pharmaceutical composition comprising a heterotandem bicyclic peptide complex comprising one or more CD137 binding peptide ligand, or a pharmaceutically acceptable salt thereof, and a pharmaceutically acceptable carrier, adjuvant, or vehicle. In some embodiments, the present invention provides a pharmaceutical composition for use in treatment of a cancer, comprising a heterotandem bicyclic peptide complex comprising one or more CD137 binding peptide ligand, or a pharmaceutically acceptable salt thereof, an immuno-oncology agent, and a pharmaceutically acceptable carrier, adjuvant, or vehicle.

In some embodiments, the present invention provides a pharmaceutical composition comprising BT7480, or a pharmaceutically acceptable salt thereof, and a pharmaceutically acceptable carrier, adjuvant, or vehicle. In some embodiments, the present invention provides a pharmaceutical composition for use in treatment of a cancer, comprising BT7480, or a pharmaceutically acceptable salt thereof, an immuno-oncology agent, and a pharmaceutically acceptable carrier, adjuvant, or vehicle.

In some embodiments, the present invention provides a pharmaceutical composition comprising BT7455, or a pharmaceutically acceptable salt thereof, and a pharmaceutically acceptable carrier, adjuvant, or vehicle. In some embodiments, the present invention provides a pharmaceutical composition for use in treatment of a cancer, comprising BT7455, or a pharmaceutically acceptable salt thereof, an immuno-oncology agent, and a pharmaceutically acceptable carrier, adjuvant, or vehicle.

In some embodiments, a composition comprises a pharmaceutically acceptable carrier, adjuvant, or vehicle. The term “pharmaceutically acceptable carrier, adjuvant, or vehicle” refers to a non-toxic carrier, adjuvant, or vehicle that does not destroy the pharmacological activity of the compound with which it is formulated. Pharmaceutically acceptable carriers, adjuvants or vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

The term “patient,” as used herein, means an animal, preferably a mammal, and most preferably a human.

Compositions of the present invention may be administered orally, parenterally, by inhalation spray, topically, rectally, nasally, buccally, vaginally or via an implanted reservoir. The term “parenteral” as used herein includes subcutaneous, intravenous, intramuscular, intra-articular, intra-synovial, intrasternal, intrathecal, intrahepatic, intralesional and intracranial injection or infusion techniques. In some embodiments, the compositions are administered orally, intraperitoneally or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added.

Alternatively, pharmaceutically acceptable compositions of this invention may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, provided pharmaceutically acceptable compositions may be formulated in a suitable ointment containing the active component suspended or dissolved in one or more carriers. Carriers for topical administration of compounds of this invention include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For ophthalmic use, provided pharmaceutically acceptable compositions may be formulated as micronized suspensions in isotonic, pH adjusted sterile saline, or, preferably, as solutions in isotonic, pH adjusted sterile saline, either with or without a preservative such as benzylalkonium chloride. Alternatively, for ophthalmic uses, the pharmaceutically acceptable compositions may be formulated in an ointment such as petrolatum.

Pharmaceutically acceptable compositions of this invention may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Pharmaceutically acceptable compositions of this invention may also be formulated for oral administration. Such formulations may be administered with or without food. In some embodiments, pharmaceutically acceptable compositions of this invention are administered without food. In other embodiments, pharmaceutically acceptable compositions of this invention are administered with food.

It should also be understood that a specific dosage and treatment regimen for any particular patient will depend upon a variety of factors, including the activity of the specific compound employed, the age, body weight, general health, sex, diet, time of administration, rate of excretion, drug combination, and the judgment of the treating physician and the severity of the particular disease being treated. The amount of a compound of the present invention in the composition will also depend upon the particular compound in the composition.

### 4. Methods for Treating Cancers

According to some embodiments, the present invention provides a method of treating a cancer in a patient, comprising administering to the patient a therapeutically effective amount of a heterotandem bicyclic peptide complex comprising one or more CD137 binding peptide ligand, or a pharmaceutically acceptable salt thereof, and an immuno-oncology agent.

In some embodiments, the present invention provides a use of a heterotandem bicyclic peptide complex comprising one or more CD137 binding peptide ligand, or a pharmaceutically acceptable salt thereof, in combination with an immuno-oncology agent, for treatment of a cancer.

In some embodiments, the present invention provides a method of treating a cancer in a patient, comprising administering to the patient a therapeutically effective amount of BT7480, or a pharmaceutically acceptable salt thereof, and an immuno-oncology agent. In some embodiments, the present invention provides a use of BT7480, or a pharmaceutically acceptable salt thereof, in combination with an immuno-oncology agent, for treatment of a cancer.

In some embodiments, the present invention provides a method of treating a cancer in a patient, comprising administering to the patient a therapeutically effective amount of BT7455, or a pharmaceutically acceptable salt thereof, and an immuno-oncology agent. In some embodiments, the present invention provides a use of BT7455, or a pharmaceutically acceptable salt thereof, in combination with an immuno-oncology agent, for treatment of a cancer.

### Exemplary Cancers

In some embodiments, the cancer is a solid tumor. In some embodiments, the cancer is associated with MT1-MMP. In some embodiments, the cancer is high MT1-MMP expressing. For example, Adley et al. have reported that MT1-MMP has a high level of expression in clear cell carcinomas of the ovary (Adley et al. “Expression of Membrane Type 1 Matrix Metalloproteinase (MMP-14) in Epithelial Ovarian Cancer: High Level Expression in Clear Cell Carcinoma” Gynecol Oncol. 2009 February; 112(2): 319-324).

In some embodiments, the cancer is associated with Nectin-4. In some embodiments, the cancer is high Nectin-4 expressing.

In some embodiments, the cancer is associated with EphA2. In some embodiments, the cancer is high EphA2 expressing.

In some embodiments, the cancer is associated with PD-Li. In some embodiments, the cancer is high PD-L1 expressing.

In some embodiments, the cancer is associated with PSMA. In some embodiments, the cancer is high PSMA expressing.

In some embodiments, the cancer is bladder cancer. In some embodiments, the bladder cancer is selected from the group consisting of basal, p53-like, and luminal.

In some embodiments, the cancer is endometrial cancer. In some embodiments, the endometrial cancer is selected from the group consisting of MMR-D, POLE EDM, p53 WT, p53 abnormal, Type I, Type II, carcinoma, carcinosarcoma, endometrioid adenocarcinoma, serous carcinoma, clear cell carcinoma, mucinous carcinoma, mixed or undifferentiated carcinoma, mixed serous and endometrioid, mixed serous and low-grade endometrioid, and undifferentiated.

In some embodiments, the cancer is esophageal cancer. In some embodiments, the esophageal cancer is selected from the group consisting of adenocarcinoma (EAC), squamous cell carcinoma (ESCC), chromosomal instability (CIN), Epstein-Barr virus (EBV), genomically stable (GS), and microsatellite instability (MSI).

In some embodiments, the cancer is glioblastoma. In some embodiments, the glioblastoma is selected from the group consisting of proneural, neural, classical, and mesenchymal.

In some embodiments, the cancer is mesothelioma. In some embodiments, the mesothelioma is selected from the group consisting of pleural mesothelioma, peritoneal mesothelioma, pericardial mesothelioma, epithelioid mesothelioma, sarcomatoid mesothelioma, biphasic mesothelioma, and malignant mesothelioma.

In some embodiments, the cancer is multiple myeloma. In some embodiments, the multiple myeloma is selected from the group consisting of hyperdiploid, non-hyperdiploid, cyclin D translocation, MMSET translocation, MAF translocation, and unclassified.

In some embodiments, the cancer is ovarian cancer. In some embodiments, the ovarian cancer is selected from the group consisting of clear cell, endometrioid, mucinous, high-grade serous and low-grade serous ovarian cancer.

In some embodiments, the cancer is pancreatic cancer. In some embodiments, the pancreatic cancer is selected from the group consisting of squamous, pancreatic progenitor, immunogenic, and ADEX (Aberrantly Differentiated Endocrine eXocrine) pancreatic cancer.

In some embodiments, the cancer is prostate cancer. In some embodiments, the prostate cancer is selected from the group consisting of AZGP1 (subtype I), MUC1 (subtype II), and MUC1 (subtype III) prostate cancer.

In some embodiments, a cancer is a lung cancer. In some embodiments, a lung cancer is a met-amplified squamous NSCLC, a squamous cell NSCLC with wild type EGFR, or a T790M EGFR-expressing lung adenocarcinoma.

In some embodiments, a cancer is a breast cancer. In some embodiments, a breast cancer is a triple negative breast cancer. In some embodiments, a breast cancer is a basaloid triple negative breast cancer.

In some embodiments, a cancer is a colon cancer. In some embodiments, a cancer is a colorectal adenocarcinoma. In some embodiments, a colorectal adenocarcinoma is a high pgp-expressing colorectal adenocarcinoma.

In some embodiments, a cancer is a gastric cancer. In some embodiments, a gastric cancer is a FGFR-amplified gastric cancer.

In some embodiments, a cancer is a head and neck cancer. In some embodiments, a head and neck cancer is a nasal septum squamous cell carcinoma.

In some embodiments, a cancer is a sarcoma. In some embodiments, a sarcoma is a fibrosarcoma. In some embodiments, a fibrosarcoma is an N-ras mutant/IDH1 mutant soft tissue sarcoma (STS).

Cancer includes, in one embodiment, without limitation, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease or non-Hodgkin's disease), Waldenstrom's macroglobulinemia, multiple myeloma, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, and retinoblastoma).

In some embodiments, the cancer is glioma, astrocytoma, glioblastoma multiforme (GBM, also known as glioblastoma), medulloblastoma, craniopharyngioma, ependymoma, pinealoma, hemangioblastoma, acoustic neuroma, oligodendroglioma, schwannoma, neurofibrosarcoma, meningioma, melanoma, neuroblastoma, or retinoblastoma.

In some embodiments, the cancer is acoustic neuroma, astrocytoma (e.g. Grade I—Pilocytic Astrocytoma, Grade II—Low-grade Astrocytoma, Grade III—Anaplastic Astrocytoma, or Grade IV—Glioblastoma (GBM)), chordoma, CNS lymphoma, craniopharyngioma, brain stem glioma, ependymoma, mixed glioma, optic nerve glioma, subependymoma, medulloblastoma, meningioma, metastatic brain tumor, oligodendroglioma, pituitary tumors, primitive neuroectodermal (PNET) tumor, or schwannoma. In some embodiments, the cancer is a type found more commonly in children than adults, such as brain stem glioma, craniopharyngioma, ependymoma, juvenile pilocytic astrocytoma (JPA), medulloblastoma, optic nerve glioma, pineal tumor, primitive neuroectodermal tumors (PNET), or rhabdoid tumor. In some embodiments, the patient is an adult human. In some embodiments, the patient is a child or pediatric patient.

Cancer includes, in another embodiment, without limitation, mesothelioma, hepatobilliary (hepatic and billiary duct), bone cancer, pancreatic cancer, skin cancer, cancer of the head or neck, cutaneous or intraocular melanoma, ovarian cancer, colon cancer, rectal cancer, cancer of the anal region, stomach cancer, gastrointestinal (gastric, colorectal, and duodenal), uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, testicular cancer, chronic or acute leukemia, chronic myeloid leukemia, lymphocytic lymphomas, cancer of the bladder, cancer of the kidney or ureter, renal cell carcinoma, carcinoma of the renal pelvis, non-Hodgkins's lymphoma, spinal axis tumors, brain stem glioma, pituitary adenoma, adrenocortical cancer, gall bladder cancer, multiple myeloma, cholangiocarcinoma, fibrosarcoma, neuroblastoma, retinoblastoma, or a combination of one or more of the foregoing cancers.

In some embodiments, the cancer is selected from hepatocellular carcinoma, ovarian cancer, ovarian epithelial cancer, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical adenoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from hepatocellular carcinoma (HCC), hepatoblastoma, colon cancer, rectal cancer, ovarian cancer, ovarian epithelial cancer, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical adenoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, a cancer is a solid tumor, such as a sarcoma, carcinoma, or lymphoma. Solid tumors generally comprise an abnormal mass of tissue that typically does not include cysts or liquid areas. In some embodiments, the cancer is selected from renal cell carcinoma, or kidney cancer; hepatocellular carcinoma (HCC) or hepatoblastoma, or liver cancer; melanoma; breast cancer; colorectal carcinoma, or colorectal cancer; colon cancer; rectal cancer; anal cancer; lung cancer, such as non-small cell lung cancer (NSCLC) or small cell lung cancer (SCLC); ovarian cancer, ovarian epithelial cancer, ovarian carcinoma, or fallopian tube cancer; papillary serous cystadenocarcinoma or uterine papillary serous carcinoma (UPSC); prostate cancer; testicular cancer; gallbladder cancer; hepatocholangiocarcinoma; soft tissue and bone synovial sarcoma; rhabdomyosarcoma; osteosarcoma; chondrosarcoma; Ewing sarcoma; anaplastic thyroid cancer; adrenocortical carcinoma; pancreatic cancer; pancreatic ductal carcinoma or pancreatic adenocarcinoma; gastrointestinal/stomach (GIST) cancer; lymphoma; squamous cell carcinoma of the head and neck (SCCHN); salivary gland cancer; glioma, or brain cancer; neurofibromatosis-1 associated malignant peripheral nerve sheath tumors (MPNST); Waldenstrom's macroglobulinemia; or medulloblastoma.

In some embodiments, the cancer is selected from renal cell carcinoma, hepatocellular carcinoma (HCC), hepatoblastoma, colorectal carcinoma, colorectal cancer, colon cancer, rectal cancer, anal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, chondrosarcoma, anaplastic thyroid cancer, adrenocortical carcinoma, pancreatic cancer, pancreatic ductal carcinoma, pancreatic adenocarcinoma, glioma, brain cancer, neurofibromatosis-1 associated malignant peripheral nerve sheath tumors (MPNST), Waldenstrom's macroglobulinemia, or medulloblastoma.

In some embodiments, the cancer is selected from hepatocellular carcinoma (HCC), hepatoblastoma, colon cancer, rectal cancer, ovarian cancer, ovarian epithelial cancer, ovarian carcinoma, fallopian tube cancer, papillary serous cystadenocarcinoma, uterine papillary serous carcinoma (UPSC), hepatocholangiocarcinoma, soft tissue and bone synovial sarcoma, rhabdomyosarcoma, osteosarcoma, anaplastic thyroid cancer, adrenocortical carcinoma, pancreatic cancer, pancreatic ductal carcinoma, pancreatic adenocarcinoma, glioma, neurofibromatosis-1 associated malignant peripheral nerve sheath tumors (MPNST), Waldenstrom's macroglobulinemia, or medulloblastoma.

In some embodiments, the cancer is hepatocellular carcinoma (HCC). In some embodiments, the cancer is hepatoblastoma. In some embodiments, the cancer is colon cancer. In some embodiments, the cancer is rectal cancer. In some embodiments, the cancer is ovarian cancer, or ovarian carcinoma. In some embodiments, the cancer is ovarian epithelial cancer. In some embodiments, the cancer is fallopian tube cancer. In some embodiments, the cancer is papillary serous cystadenocarcinoma. In some embodiments, the cancer is uterine papillary serous carcinoma (UPSC). In some embodiments, the cancer is hepatocholangiocarcinoma. In some embodiments, the cancer is soft tissue and bone synovial sarcoma. In some embodiments, the cancer is rhabdomyosarcoma. In some embodiments, the cancer is osteosarcoma. In some embodiments, the cancer is anaplastic thyroid cancer. In some embodiments, the cancer is adrenocortical carcinoma. In some embodiments, the cancer is pancreatic cancer, or pancreatic ductal carcinoma. In some embodiments, the cancer is pancreatic adenocarcinoma. In some embodiments, the cancer is glioma. In some embodiments, the cancer is malignant peripheral nerve sheath tumors (MPNST). In some embodiments, the cancer is neurofibromatosis-1 associated MPNST. In some embodiments, the cancer is Waldenstrom's macroglobulinemia. In some embodiments, the cancer is medulloblastoma.

In some embodiments, a cancer is a viral-associated cancer, including human immunodeficiency virus (HIV) associated solid tumors, human papilloma virus (HPV)-16 positive incurable solid tumors, and adult T-cell leukemia, which is caused by human T-cell leukemia virus type I (HTLV-I) and is a highly aggressive form of CD4+ T-cell leukemia characterized by clonal integration of HTLV-I in leukemic cells (See https://clinicaltrials.gov/ct2/show/study/NCT02631746); as well as virus-associated tumors in gastric cancer, nasopharyngeal carcinoma, cervical cancer, vaginal cancer, vulvar cancer, squamous cell carcinoma of the head and neck, and Merkel cell carcinoma. (See https://clinicaltrials.gov/ct2/show/study/NCT02488759; see also https://clinicaltrials.gov/ct2/show/study/NCT0240886; https://clinicaltrials.gov/ct2/show/NCT02426892)

In some embodiments, a cancer is melanoma cancer. In some embodiments, a cancer is breast cancer. In some embodiments, a cancer is lung cancer. In some embodiments, a cancer is small cell lung cancer (SCLC). In some embodiments, a cancer is non-small cell lung cancer (NSCLC).

In some embodiments, a cancer is treated by arresting further growth of the tumor. In some embodiments, a cancer is treated by reducing the size (e.g., volume or mass) of the tumor by at least 5%, 10%, 25%, 50%, 75%, 90% or 99% relative to the size of the tumor prior to treatment. In some embodiments, a cancer is treated by reducing the quantity of the tumor in the patient by at least 5%, 10%, 25%, 50%, 75%, 90% or 99% relative to the quantity of the tumor prior to treatment.

The heterotandem bicyclic peptide complexes and compositions, according to the method of the present invention, may be administered using any amount and any route of administration effective for treating or lessening the severity of a cancer. The exact amount required will vary from subject to subject, depending on the species, age, and general condition of the subject, the severity of the disease or condition, the particular agent, its mode of administration, and the like. The heterotandem bicyclic peptide complexes are preferably formulated in dosage unit form for ease of administration and uniformity of dosage. The expression “dosage unit form” as used herein refers to a physically discrete unit of agent appropriate for the patient to be treated. It will be understood, however, that the total daily usage of the heterotandem bicyclic peptide complexes and compositions of the present invention will be decided by the attending physician within the scope of sound medical judgment. The specific effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed, and like factors well known in the medical arts. The term “patient”, as used herein, means an animal, preferably a mammal, and most preferably a human.

Pharmaceutically acceptable compositions of this invention can be administered to humans and other animals orally, rectally, parenterally, intracisternally, intravaginally, intraperitoneally, topically (as by powders, ointments, or drops), bucally, as an oral or nasal spray, or the like, depending on the severity of the disease or disorder being treated. In certain embodiments, the heterotandem bicyclic peptide complexes of the invention may be administered orally or parenterally at dosage levels of about 0.01 mg/kg to about 100 mg/kg, or from about 1 mg/kg to about 25 mg/kg, of subject body weight per day, one or more times a day, to obtain the desired therapeutic effect.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a compound of the present invention, it is often desirable to slow the absorption of the compound from subcutaneous or intramuscular injection. This may be accomplished by the use of a liquid suspension of crystalline or amorphous material with poor water solubility. The rate of absorption of the compound then depends upon its rate of dissolution that, in turn, may depend upon crystal size and crystalline form. Alternatively, delayed absorption of a parenterally administered compound form is accomplished by dissolving or suspending the compound in an oil vehicle. Injectable depot forms are made by forming microencapsule matrices of the compound in biodegradable polymers such as polylactide-polyglycolide. Depending upon the ratio of compound to polymer and the nature of the particular polymer employed, the rate of compound release can be controlled. Examples of other biodegradable polymers include poly(orthoesters) and poly(anhydrides). Depot injectable formulations are also prepared by entrapping the compound in liposomes or microemulsions that are compatible with body tissues.

Compositions for rectal or vaginal administration are preferably suppositories which can be prepared by mixing the heterotandem bicyclic peptide complexes of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polyethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes.

Dosage forms for topical or transdermal administration of a heterotandem bicyclic peptide complex comprising one or more CD137 binding peptide ligand, for example, as described herein, include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, and eye drops are also contemplated as being within the scope of this invention. Additionally, the present invention contemplates the use of transdermal patches, which have the added advantage of providing controlled delivery of a compound to the body. Such dosage forms can be made by dissolving or dispensing the compound in the proper medium. Absorption enhancers can also be used to increase the flux of the compound across the skin. The rate can be controlled by either providing a rate controlling membrane or by dispersing the compound in a polymer matrix or gel.

### Co-Administration of a Heterotandem Bicyclic Peptide Complex and an Immuno-Oncology Agent

A heterotandem bicyclic peptide complex, for example, as described herein, and an immuno-oncology agent may be administered separately, as part of a multiple dosage regimen. Alternatively, a heterotandem bicyclic peptide complex, for example, as described herein, and an immuno-oncology agent may be mixed together in a single composition as a single dosage form. In some embodiments, a heterotandem bicyclic peptide complex is BT7480 or BT7455, or a pharmaceutically acceptable salt thereof.

In some embodiments, a heterotandem bicyclic peptide complex, for example, as described herein, is administered separately from an immuno-oncology agent. In some embodiments, a heterotandem bicyclic peptide complex, for example, as described herein, and an immuno-oncology agent are administered simultaneously. In some embodiments, a heterotandem bicyclic peptide complex, for example, as described herein, and an immuno-oncology agent are administered sequentially. In some embodiments, a heterotandem bicyclic peptide complex, for example, as described herein, and an immuno-oncology agent are administered within a period of time from one another, for example within 1, 2, 3, 4, 5, 6, 7, 8, 9, 10, 11, 12, 13, 14, 15, 16, 17, 18, 18, 20, 21, 22, 23, or 24 hours from one another. In some embodiments, a heterotandem bicyclic peptide complex, for example, as described herein, and an immuno-oncology agent are administered within greater than 24 hours apart. In some embodiments, a heterotandem bicyclic peptide complex, for example, as described herein, and an immuno-oncology agent are administered within 1, 2, 3, 4, 5, 6, or 7 days from one another. In some embodiments, a heterotandem bicyclic peptide complex, for example, as described herein, and an immuno-oncology agent are administered within greater than one week apart. In some embodiments, a heterotandem bicyclic peptide complex, for example, as described herein, and an immuno-oncology agent are administered within 1, 2, 3, 4, or 5 weeks from one another.

As used herein, the term “combination,” “combined,” and related terms refers to the simultaneous or sequential administration of therapeutic agents in accordance with this invention. For example, a heterotandem bicyclic peptide complex, for example, as described herein, may be administered with an immuno-oncology agent simultaneously or sequentially in separate unit dosage forms or together in a single unit dosage form. Accordingly, in some embodiments, the present invention provides a single unit dosage form comprising a heterotandem bicyclic peptide complex, for example, as described herein, an immuno-oncology agent, and optionally a pharmaceutically acceptable carrier, adjuvant, or vehicle.

The amount of a heterotandem bicyclic peptide complex, for example, as described herein, and an immuno-oncology agent that may be combined with the carrier materials to produce a single dosage form will vary depending upon the host treated and the particular mode of administration. Preferably, a composition of the invention should be formulated so that a dosage of between 0.001-100 mg/kg body weight/day of a heterotandem bicyclic peptide complex, for example, as described herein, can be administered.

A heterotandem bicyclic peptide complex comprising one or more CD137 binding peptide ligand, for example, as described herein, and an immuno-oncology agent may act synergistically. Therefore, the amount of a heterotandem bicyclic peptide complex, for example, as described herein, and an immuno-oncology agent in such compositions may be less than that required in a monotherapy utilizing only that therapeutic agent.

The amount of an immuno-oncology agent present in the compositions of this invention may be no more than the amount that would normally be administered in a composition comprising it as the only active agent. Preferably the amount of an immuno-oncology agent in the presently disclosed compositions will range from about 50% to 100% of the amount normally present in a composition comprising that agent as the only therapeutically active agent. In some embodiments, an immuno-oncology agent is administered at a dosage of about 50%, about 55%, about 60%, about 65%, about 70%, about 75%, about 80%, about 85%, about 90%, or about 95% of the amount normally administered as monotherapy. As used herein, the phrase “normally administered” means the amount an FDA approved therapeutic agent is approved for dosing per the FDA label insert.

The pharmaceutical compositions of this invention may also be incorporated into compositions for coating an implantable medical device, such as prostheses, artificial valves, vascular grafts, stents and catheters. Vascular stents, for example, have been used to overcome restenosis (re-narrowing of the vessel wall after injury). However, patients using stents or other implantable devices risk clot formation or platelet activation. These unwanted effects may be prevented or mitigated by pre-coating the device with a pharmaceutically acceptable composition comprising a kinase inhibitor. Implantable devices coated with a compound of this invention are another embodiment of the present invention.

### 5. Exemplary Immuno-Oncology Agents

As used herein, the term “an immuno-oncology agent” refers to an agent which is effective to enhance, stimulate, and/or up-regulate immune responses in a subject. In some embodiments, the administration of an immuno-oncology agent with a heterotandem bicyclic peptide complex comprising one or more CD137 binding peptide ligand, for example, as described herein, has a synergic effect in treating a cancer.

An immuno-oncology agent can be, for example, a small molecule drug, an antibody, or a biologic or small molecule. Examples of biologic immuno-oncology agents include, but are not limited to, cancer vaccines, antibodies, and cytokines. In some embodiments, an antibody is a monoclonal antibody. In some embodiments, a monoclonal antibody is humanized or human.

In some embodiments, an immuno-oncology agent is (i) an agonist of a stimulatory (including a co-stimulatory) receptor or (ii) an antagonist of an inhibitory (including a co-inhibitory) signal on T cells, both of which result in amplifying antigen-specific T cell responses.

Certain of the stimulatory and inhibitory molecules are members of the immunoglobulin super family (IgSF). One important family of membrane-bound ligands that bind to co-stimulatory or co-inhibitory receptors is the B7 family, which includes B7-1, B7-2, B7-H1 (PD-L1), B7-DC (PD-L2), B7-H2 (ICOS-L), B7-H3, B7-H4, B7-H5 (VISTA), and B7-H6. Another family of membrane bound ligands that bind to co-stimulatory or co-inhibitory receptors is the TNF family of molecules that bind to cognate TNF receptor family members, which includes CD40 and CD40L, OX-40, OX-40L, CD70, CD27L, CD30, CD30L, 4-1BBL, CD137 (4-1BB), TRAIL/Apo2-L, TRAILR1/DR4, TRAILR2/DR5, TRAILR3, TRAILR4, OPG, RANK, RANKL, TWEAKR/Fn14, TWEAK, BAFFR, EDAR, XEDAR, TACI, APRIL, BCMA, LTβR, LIGHT, DcR3, HVEM, VEGI/TL1A, TRAMP/DR3, EDAR, EDA1, XEDAR, EDA2, TNFR1, Lymphotoxin α/TNFβ, TNFR2, TNFα, LTβR, Lymphotoxin α1β2, FAS, FASL, RELT, DR6, TROY, NGFR.

In some embodiments, an immuno-oncology agent is a cytokine that inhibits T cell activation (e.g., IL-6, IL-10, TGF-β, VEGF, and other immunosuppressive cytokines) or a cytokine that stimulates T cell activation, for stimulating an immune response.

In some embodiments, a combination of a heterotandem bicyclic peptide complex comprising one or more CD137 binding peptide ligand, for example, as described herein, and an immuno-oncology agent can stimulate T cell responses. In some embodiments, a heterotandem bicyclic peptide complex is BT7480 or BT7455, or a pharmaceutically acceptable salt thereof. In some embodiments, an immuno-oncology agent is: (i) an antagonist of a protein that inhibits T cell activation (e.g., immune checkpoint inhibitors) such as CTLA-4, PD-1, PD-L1, PD-L2, LAG-3, TIM-3, Galectin 9, CEACAM-1, BTLA, CD69, Galectin-1, TIGIT, CD113, GPR56, VISTA, 2B4, CD48, GARP, PD1H, LAIR1, TIM-1, and TIM-4; or (ii) an agonist of a protein that stimulates T cell activation such as B7-1, B7-2, CD28, 4-1BB (CD137), 4-1BBL, ICOS, ICOS-L, OX40, OX40L, GITR, GITRL, CD70, CD27, CD40, DR3 and CD28H.

In some embodiments, an immuno-oncology agent is an antagonist of inhibitory receptors on NK cells or an agonist of activating receptors on NK cells. In some embodiments, an immuno-oncology agent is an antagonist of KIR, such as lirilumab.

In some embodiments, an immuno-oncology agent is an agent that inhibits or depletes macrophages or monocytes, including but not limited to CSF-1R antagonists such as CSF-1R antagonist antibodies including RG7155 (WO11/70024, WO11/107553, WO11/131407, WO13/87699, WO13/119716, WO13/132044) or FPA-008 (WO11/140249; WO13169264; WO14/036357).

In some embodiments, an immuno-oncology agent is selected from agonistic agents that ligate positive costimulatory receptors, blocking agents that attenuate signaling through inhibitory receptors, antagonists, and one or more agents that increase systemically the frequency of anti-tumor T cells, agents that overcome distinct immune suppressive pathways within the tumor microenvironment (e.g., block inhibitory receptor engagement (e.g., PD-L1/PD-1 interactions), deplete or inhibit Tregs (e.g., using an anti-CD25 monoclonal antibody (e.g., daclizumab) or by ex vivo anti-CD25 bead depletion), inhibit metabolic enzymes such as IDO, or reverse/prevent T cell energy or exhaustion) and agents that trigger innate immune activation and/or inflammation at tumor sites.

In some embodiments, an immuno-oncology agent is a CTLA-4 antagonist. In some embodiments, a CTLA-4 antagonist is an antagonistic CTLA-4 antibody. In some embodiments, an antagonistic CTLA-4 antibody is YERVOY (ipilimumab) or tremelimumab.

In some embodiments, an immuno-oncology agent is a PD-1 antagonist. In some embodiments, a PD-1 antagonist is administered by infusion. In some embodiments, an immuno-oncology agent is an antibody or an antigen-binding portion thereof that binds specifically to a Programmed Death-1 (PD-1) receptor and inhibits PD-1 activity. In some embodiments, a PD-1 antagonist is an antagonistic PD-1 antibody. In some embodiments, an antagonistic PD-1 antibody is OPDIVO (nivolumab), KEYTRUDA (pembrolizumab), or MEDI-0680 (AMP-514; WO2012/145493). In some embodiments, an immuno-oncology agent may be pidilizumab (CT-011). In some embodiments, an immuno-oncology agent is a recombinant protein composed of the extracellular domain of PD-L2 (B7-DC) fused to the Fc portion of IgG1, called AMP-224.

In some embodiments, an immuno-oncology agent is a PD-L1 antagonist. In some embodiments, a PD-L1 antagonist is an antagonistic PD-L1 antibody. In some embodiments, a PD-L1 antibody is MPDL3280A (RG7446; WO2010/077634), durvalumab (MEDI4736), BMS-936559 (WO2007/005874), and MSB0010718C (WO2013/79174).

In some embodiments, an immuno-oncology agent is a LAG-3 antagonist. In some embodiments, a LAG-3 antagonist is an antagonistic LAG-3 antibody. In some embodiments, a LAG3 antibody is BMS-986016 (WO10/19570, WO14/08218), or IMP-731 or IMP-321 (WO08/132601, WO009/44273).

In some embodiments, an immuno-oncology agent is a CD137 (4-1BB) agonist. In some embodiments, a CD137 (4-1BB) agonist is an agonistic CD137 antibody. In some embodiments, a CD137 antibody is urelumab or PF-05082566 (WO12/32433).

In some embodiments, an immuno-oncology agent is a GITR agonist. In some embodiments, a GITR agonist is an agonistic GITR antibody. In some embodiments, a GITR antibody is BMS-986153, BMS-986156, TRX-518 (WO006/105021, WO009/009116), or MK-4166 (WO11/028683).

In some embodiments, an immuno-oncology agent is an indoleamine (2,3)-dioxygenase (IDO) antagonist. In some embodiments, an IDO antagonist is selected from epacadostat (INCB024360, Incyte); indoximod (NLG-8189, NewLink Genetics Corporation); capmanitib (INC280, Novartis); GDC-0919 (Genentech/Roche); PF-06840003 (Pfizer); BMS:F001287 (Bristol-Myers Squibb); Phy906/KD108 (Phytoceutica); an enzyme that breaks down kynurenine (Kynase, Ikena Oncology, formerly known as Kyn Therapeutics); and NLG-919 (WO09/73620, WO009/1156652, WO11/56652, WO12/142237).

In some embodiments, an immuno-oncology agent is an OX40 agonist. In some embodiments, an OX40 agonist is an agonistic OX40 antibody. In some embodiments, an OX40 antibody is MEDI-6383 or MEDI-6469.

In some embodiments, an immuno-oncology agent is an OX40L antagonist. In some embodiments, an OX40L antagonist is an antagonistic OX40 antibody. In some embodiments, an OX40L antagonist is RG-7888 (WO06/029879).

In some embodiments, an immuno-oncology agent is a CD40 agonist. In some embodiments, a CD40 agonist is an agonistic CD40 antibody. In some embodiments, an immuno-oncology agent is a CD40 antagonist. In some embodiments, a CD40 antagonist is an antagonistic CD40 antibody. In some embodiments, a CD40 antibody is lucatumumab or dacetuzumab.

In some embodiments, an immuno-oncology agent is a CD27 agonist. In some embodiments, a CD27 agonist is an agonistic CD27 antibody. In some embodiments, a CD27 antibody is varlilumab.

In some embodiments, an immuno-oncology agent is MGA271 (to B7H3) (WO11/109400).

In some embodiments, an immuno-oncology agent is abagovomab, adecatumumab, afutuzumab, alemtuzumab, anatumomab mafenatox, apolizumab, atezolimab, avelumab, blinatumomab, BMS-936559, catumaxomab, durvalumab, epacadostat, epratuzumab, indoximod, inotuzumab ozogamicin, intelumumab, ipilimumab, isatuximab, lambrolizumab, MED14736, MPDL3280A, nivolumab, obinutuzumab, ocaratuzumab, ofatumumab, olatatumab, pembrolizumab, pidilizumab, rituximab, ticilimumab, samalizumab, or tremelimumab.

In some embodiments, an immuno-oncology agent is an immunostimulatory agent. For example, antibodies blocking the PD-1 and PD-L1 inhibitory axis can unleash activated tumor-reactive T cells and have been shown in clinical trials to induce durable anti-tumor responses in increasing numbers of tumor histologies, including some tumor types that conventionally have not been considered immunotherapy sensitive. See, e.g., Okazaki, T. et al. (2013) Nat. Immunol. 14, 1212-1218; Zou et al. (2016) Sci. Transl. Med. 8. The anti-PD-1 antibody nivolumab (OPDIVO®, Bristol-Myers Squibb, also known as ONO-4538, MDX1106 and BMS-936558), has shown potential to improve the overall survival in patients with RCC who had experienced disease progression during or after prior anti-angiogenic therapy.

In some embodiments, the immunomodulatory therapeutic specifically induces apoptosis of tumor cells. Approved immunomodulatory therapeutics which may be used in the present invention include pomalidomide (POMALYST®, Celgene); lenalidomide (REVLIMID®, Celgene); ingenol mebutate (PICATO®, LEO Pharma).

In some embodiments, an immuno-oncology agent is a cancer vaccine. In some embodiments, the cancer vaccine is selected from sipuleucel-T (PROVENGE®, Dendreon/Valeant Pharmaceuticals), which has been approved for treatment of asymptomatic, or minimally symptomatic metastatic castrate-resistant (hormone-refractory) prostate cancer; and talimogene laherparepvec (IMLYGIC®, BioVex/Amgen, previously known as T-VEC), a genetically modified oncolytic viral therapy approved for treatment of unresectable cutaneous, subcutaneous and nodal lesions in melanoma. In some embodiments, an immuno-oncology agent is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (REOLYSIN®, Oncolytics Biotech), a variant of respiratory enteric orphan virus (reovirus) which does not replicate in cells that are not RAS-activated, in numerous cancers, including colorectal cancer (NCT01622543); prostate cancer (NCT01619813); head and neck squamous cell cancer (NCT01166542); pancreatic adenocarcinoma (NCT00998322); and non-small cell lung cancer (NSCLC) (NCT 00861627); enadenotucirev (NG-348, PsiOxus, formerly known as ColoAd1), an adenovirus engineered to express a full length CD80 and an antibody fragment specific for the T-cell receptor CD3 protein, in ovarian cancer (NCT02028117); metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676); and peritoneal disease, colorectal cancer or ovarian cancer (NCT02963831); GL-ONC1 (GLV-1 h68/GLV-1 h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260); fallopian tube cancer, ovarian cancer (NCT 02759588); or CG0070 (Cold Genesys), an adenovirus engineered to express GM-CSF, in bladder cancer (NCT02365818).

In some embodiments, an immuno-oncology agent is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TG01 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response.

In some embodiments, an immuno-oncology agent is a T-cell engineered to express a chimeric antigen receptor, or CAR. The T-cells engineered to express such chimeric antigen receptor are referred to as a CAR-T cells.

CARs have been constructed that consist of binding domains, which may be derived from natural ligands, single chain variable fragments (scFv) derived from monoclonal antibodies specific for cell-surface antigens, fused to endodomains that are the functional end of the T-cell receptor (TCR), such as the CD3-zeta signaling domain from TCRs, which is capable of generating an activation signal in T lymphocytes. Upon antigen binding, such CARs link to endogenous signaling pathways in the effector cell and generate activating signals similar to those initiated by the TCR complex.

For example, in some embodiments the CAR-T cell is one of those described in U.S. Pat. No. 8,906,682 (June et al.; hereby incorporated by reference in its entirety), which discloses CAR-T cells engineered to comprise an extracellular domain having an antigen binding domain (such as a domain that binds to CD19), fused to an intracellular signaling domain of the T cell antigen receptor complex zeta chain (such as CD3 zeta). When expressed in the T cell, the CAR is able to redirect antigen recognition based on the antigen binding specificity. In the case of CD19, the antigen is expressed on malignant B cells. Over 200 clinical trials are currently in progress employing CAR-T in a wide range of indications. [https://clinicaltrials. gov/ct2/results?term=chimeric+antigen+receptors&pg=1].

In some embodiments, an immunostimulatory agent is an activator of retinoic acid receptor-related orphan receptor γ (RORγt). RORγt is a transcription factor with key roles in the differentiation and maintenance of Type 17 effector subsets of CD4+(Th17) and CD8+(Tc17) T cells, as well as the differentiation of IL-17 expressing innate immune cell subpopulations such as NK cells. In some embodiments, an activator of RORγt is LYC-55716 (Lycera), which is currently being evaluated in clinical trials for the treatment of solid tumors (NCT02929862).

In some embodiments, an immunostimulatory agent is an agonist or activator of a toll-like receptor (TLR). Suitable activators of TLRs include an agonist or activator of TLR9 such as SD-101 (Dynavax). SD-101 is an immunostimulatory CpG which is being studied for B-cell, follicular and other lymphomas (NCT02254772). Agonists or activators of TLR8 which may be used in the present invention include motolimod (VTX-2337, VentiRx Pharmaceuticals) which is being studied for squamous cell cancer of the head and neck (NCT02124850) and ovarian cancer (NCT02431559).

Other immuno-oncology agents that can be used in the present invention include urelumab (BMS-663513, Bristol-Myers Squibb), an anti-CD137 monoclonal antibody; varlilumab (CDX-1127, Celldex Therapeutics), an anti-CD27 monoclonal antibody; BMS-986178 (Bristol-Myers Squibb), an anti-OX40 monoclonal antibody; lirilumab (IPH2102/BMS-986015, Innate Pharma, Bristol-Myers Squibb), an anti-KIR monoclonal antibody; monalizumab (IPH2201, Innate Pharma, AstraZeneca) an anti-NKG2A monoclonal antibody; andecaliximab (GS-5745, Gilead Sciences), an anti-MMP9 antibody; MK-4166 (Merck & Co.), an anti-GITR monoclonal antibody.

In some embodiments, an immunostimulatory agent is selected from elotuzumab, mifamurtide, an agonist or activator of a toll-like receptor, and an activator of RORγt.

In some embodiments, an immunostimulatory therapeutic is recombinant human interleukin 15 (rhIL-15). rhIL-15 has been tested in the clinic as a therapy for melanoma and renal cell carcinoma (NCT01021059 and NCT01369888) and leukemias (NCT02689453). In some embodiments, an immunostimulatory agent is recombinant human interleukin 12 (rhIL-12). In some embodiments, an IL-15 based immunotherapeutic is heterodimeric IL-15 (hetIL-15, Novartis/Admune), a fusion complex composed of a synthetic form of endogenous IL-15 complexed to the soluble IL-15 binding protein IL-15 receptor alpha chain (IL15:sIL-15RA), which has been tested in Phase 1 clinical trials for melanoma, renal cell carcinoma, non-small cell lung cancer and head and neck squamous cell carcinoma (NCT02452268). In some embodiments, a recombinant human interleukin 12 (rhIL-12) is NM-IL-12 (Neumedicines, Inc.), NCT02544724, or NCT02542124.

In some embodiments, an immuno-oncology agent is selected from those descripted in Jerry L. Adams et al., “Big opportunities for small molecules in immuno-oncology,” Cancer Therapy 2015, Vol. 14, pages 603-622, the content of which is incorporated herein by reference in its entirety. In some embodiments, an immuno-oncology agent is selected from the examples described in Table 1 of Jerry L. Adams et al. In some embodiments, an immuno-oncology agent is a small molecule targeting an immuno-oncology target selected from those listed in Table 2 of Jerry L. Adams et al. In some embodiments, an immuno-oncology agent is a small molecule agent selected from those listed in Table 2 of Jerry L. Adams et al.

In some embodiments, an immuno-oncology agent is selected from the small molecule immuno-oncology agents described in Peter L. Toogood, “Small molecule immuno-oncology therapeutic agents,” Bioorganic & Medicinal Chemistry Letters 2018, Vol. 28, pages 319-329, the content of which is incorporated herein by reference in its entirety. In some embodiments, an immuno-oncology agent is an agent targeting the pathways as described in Peter L. Toogood.

In some embodiments, an immuno-oncology agent is selected from those described in Sandra L. Ross et al., “Bispecific T cell engager (BITE®) antibody constructs can mediate bystander tumor cell killing”, PLoS ONE 12(8): e0183390, the content of which is incorporated herein by reference in its entirety. In some embodiments, an immuno-oncology agent is a bispecific T cell engager (BITE®) antibody construct. In some embodiments, a bispecific T cell engager (BITE®) antibody construct is a CD19/CD3 bispecific antibody construct. In some embodiments, a bispecific T cell engager (BITE®) antibody construct is an EGFR/CD3 bispecific antibody construct. In some embodiments, a bispecific T cell engager (BITE®) antibody construct activates T cells. In some embodiments, a bispecific T cell engager (BITE®) antibody construct activates T cells, which release cytokines inducing upregulation of intercellular adhesion molecule 1 (ICAM-1) and FAS on bystander cells. In some embodiments, a bispecific T cell engager (BITE®) antibody construct activates T cells which result in induced bystander cell lysis. In some embodiments, the bystander cells are in solid tumors. In some embodiments, the bystander cells being lysed are in proximity to the BITE®-activated T cells. In some embodiments, the bystander cells comprises tumor-associated antigen (TAA) negative cancer cells. In some embodiment, the bystander cells comprise EGFR-negative cancer cells. In some embodiments, an immuno-oncology agent is an antibody which blocks the PD-L1/PD1 axis and/or CTLA4. In some embodiments, an immuno-oncology agent is an ex vivo expanded tumor-infiltrating T cell. In some embodiments, an immuno-oncology agent is a bispecific antibody construct or chimeric antigen receptors (CARs) that directly connect T cells with tumor-associated surface antigens (TAAs).

### Exemplary Checkpoint Inhibitors

In some embodiments, an immuno-oncology agent is an immune checkpoint inhibitor as described herein.

The term “checkpoint inhibitor” as used herein relates to agents useful in preventing cancer cells from avoiding the immune system of the patient. One of the major mechanisms of anti-tumor immunity subversion is known as “T-cell exhaustion,” which results from chronic exposure to antigens that has led to up-regulation of inhibitory receptors. These inhibitory receptors serve as immune checkpoints in order to prevent uncontrolled immune reactions.

PD-1 and co-inhibitory receptors such as cytotoxic T-lymphocyte antigen 4 (CTLA-4, B and T Lymphocyte Attenuator (BTLA; CD272), T cell Immunoglobulin and Mucin domain-3 (Tim-3), Lymphocyte Activation Gene-3 (Lag-3; CD223), and others are often referred to as a checkpoint regulators. They act as molecular “gatekeepers” that allow extracellular information to dictate whether cell cycle progression and other intracellular signaling processes should proceed.

In some embodiments, an immune checkpoint inhibitor is an antibody to PD-1. PD-1 binds to the programmed cell death 1 receptor (PD-1) to prevent the receptor from binding to the inhibitory ligand PDL-1, thus overriding the ability of tumors to suppress the host anti-tumor immune response.

In some embodiments, the checkpoint inhibitor is a biologic therapeutic or a small molecule. In some embodiments, the checkpoint inhibitor is a monoclonal antibody, a humanized antibody, a fully human antibody, a fusion protein or a combination thereof. In some embodiments, the checkpoint inhibitor inhibits a checkpoint protein selected from CTLA-4, PDL 1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor interacts with a ligand of a checkpoint protein selected from CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, B-7 family ligands or a combination thereof. In some embodiments, the checkpoint inhibitor is an immunostimulatory agent, a T cell growth factor, an interleukin, an antibody, a vaccine or a combination thereof. In some embodiments, the interleukin is IL-7 or IL-15. In some embodiments, the interleukin is glycosylated IL-7. In an additional aspect, the vaccine is a dendritic cell (DC) vaccine.

Checkpoint inhibitors include any agent that blocks or inhibits in a statistically significant manner, the inhibitory pathways of the immune system. Such inhibitors can include small molecule inhibitors or can include antibodies, or antigen binding fragments thereof, that bind to and block or inhibit immune checkpoint receptors or antibodies that bind to and block or inhibit immune checkpoint receptor ligands. Illustrative checkpoint molecules that can be targeted for blocking or inhibition include, but are not limited to, CTLA-4, PDL1, PDL2, PD1, B7-H3, B7-H4, BTLA, HVEM, GAL9, LAG3, TIM3, VISTA, KIR, 2B4 (belongs to the CD2 family of molecules and is expressed on all NK, T6, and memory CD8+(αβ) T cells), CD160 (also referred to as BY55), CGEN-15049, CHK 1 and CHK2 kinases, A2aR, and various B-7 family ligands. B7 family ligands include, but are not limited to, B7-1, B7-2, B7-DC, B7-H1, B7-H2, B7-H3, B7-H4, B7-H5, B7-H6 and B7-H7. Checkpoint inhibitors include antibodies, or antigen binding fragments thereof, other binding proteins, biologic therapeutics, or small molecules, that bind to and block or inhibit the activity of one or more of CTLA-4, PDL1, PDL2, PD1, BTLA, HVEM, TIM3, GAL9, LAG3, VISTA, KIR, 2B4, CD 160 and CGEN-15049. Illustrative immune checkpoint inhibitors include, but are not limited to, Tremelimumab (CTLA-4 blocking antibody), anti-OX40, PD-L1 monoclonal Antibody (Anti-B7-H1; MEDI4736), MK-3475 (PD-1 blocker), Nivolumab (anti-PD1 antibody), CT-011 (anti-PD1 antibody), BY55 monoclonal antibody, AMP224 (anti-PDL1 antibody), BMS-936559 (anti-PDL1 antibody), MPLDL3280A (anti-PDL1 antibody), MSB0010718C (anti-PDL1 antibody), and ipilimumab (anti-CTLA-4 checkpoint inhibitor). Checkpoint protein ligands include, but are not limited to PD-L1, PD-L2, B7-H3, B7-H4, CD28, CD86 and TIM-3.

In certain embodiments, the immune checkpoint inhibitor is selected from a PD-1 antagonist, a PD-L1 antagonist, and a CTLA-4 antagonist. In some embodiments, the checkpoint inhibitor is selected from the group consisting of nivolumab (OPDIVO®), ipilimumab (YERVOY®), and pembrolizumab (KEYTRUDA®). In some embodiments, the checkpoint inhibitor is selected from nivolumab (anti-PD-1 antibody, OPDIVO®, Bristol-Myers Squibb); pembrolizumab (anti-PD-1 antibody, KEYTRUDA®, Merck); ipilimumab (anti-CTLA-4 antibody, YERVOY®, Bristol-Myers Squibb); durvalumab (anti-PD-L1 antibody, IMFINZI®, AstraZeneca); and atezolizumab (anti-PD-L1 antibody, TECENTRIQ®, Genentech).

In some embodiments, the checkpoint inhibitor is selected from the group consisting of lambrolizumab (MK-3475), nivolumab (BMS-936558), pidilizumab (CT-011), AMP-224, MDX-1105, MEDI4736, MPDL3280A, BMS-936559, ipilimumab, lirlumab, IPH2101, pembrolizumab (KEYTRUDA®), and tremelimumab.

In some embodiments, an immune checkpoint inhibitor is REGN2810 (Regeneron), an anti-PD-1 antibody tested in patients with basal cell carcinoma (NCT03132636); NSCLC (NCT03088540); cutaneous squamous cell carcinoma (NCT02760498); lymphoma (NCT02651662); and melanoma (NCT03002376); pidilizumab (CureTech), also known as CT-011, an antibody that binds to PD-1, in clinical trials for diffuse large B-cell lymphoma and multiple myeloma; avelumab (BAVENCIO®, Pfizer/Merck KGaA), also known as MSB0010718C), a fully human IgG1 anti-PD-L1 antibody, in clinical trials for non-small cell lung cancer, Merkel cell carcinoma, mesothelioma, solid tumors, renal cancer, ovarian cancer, bladder cancer, head and neck cancer, and gastric cancer; or PDR001 (Novartis), an inhibitory antibody that binds to PD-1, in clinical trials for non-small cell lung cancer, melanoma, triple negative breast cancer and advanced or metastatic solid tumors. Tremelimumab (CP-675,206; Astrazeneca) is a fully human monoclonal antibody against CTLA-4 that has been in studied in clinical trials for a number of indications, including: mesothelioma, colorectal cancer, kidney cancer, breast cancer, lung cancer and non-small cell lung cancer, pancreatic ductal adenocarcinoma, pancreatic cancer, germ cell cancer, squamous cell cancer of the head and neck, hepatocellular carcinoma, prostate cancer, endometrial cancer, metastatic cancer in the liver, liver cancer, large B-cell lymphoma, ovarian cancer, cervical cancer, metastatic anaplastic thyroid cancer, urothelial cancer, fallopian tube cancer, multiple myeloma, bladder cancer, soft tissue sarcoma, and melanoma. AGEN-1884 (Agenus) is an anti-CTLA4 antibody that is being studied in Phase 1 clinical trials for advanced solid tumors (NCT02694822).

In some embodiments, a checkpoint inhibitor is an inhibitor of T-cell immunoglobulin mucin containing protein-3 (TIM-3). TIM-3 inhibitors that may be used in the present invention include TSR-022, LY3321367 and MBG453. TSR-022 (Tesaro) is an anti-TIM-3 antibody which is being studied in solid tumors (NCT02817633). LY3321367 (Eli Lilly) is an anti-TIM-3 antibody which is being studied in solid tumors (NCT03099109). MBG453 (Novartis) is an anti-TIM-3 antibody which is being studied in advanced malignancies (NCT02608268).

In some embodiments, a checkpoint inhibitor is an inhibitor of T cell immunoreceptor with Ig and ITIM domains, or TIGIT, an immune receptor on certain T cells and NK cells. TIGIT inhibitors that may be used in the present invention include BMS-986207 (Bristol-Myers Squibb), an anti-TIGIT monoclonal antibody (NCT02913313); OMP-313M32 (Oncomed); and anti-TIGIT monoclonal antibody (NCT03119428).

In some embodiments, a checkpoint inhibitor is an inhibitor of Lymphocyte Activation Gene-3 (LAG-3). LAG-3 inhibitors that may be used in the present invention include BMS-986016 and REGN3767 and IMP321. BMS-986016 (Bristol-Myers Squibb), an anti-LAG-3 antibody, is being studied in glioblastoma and gliosarcoma (NCT02658981). REGN3767 (Regeneron), is also an anti-LAG-3 antibody, and is being studied in malignancies (NCT03005782). IP321 (Immutep S.A.) is an LAG-3-Ig fusion protein, being studied in melanoma (NCT02676869); adenocarcinoma (NCT02614833); and metastatic breast cancer (NCT00349934).

Checkpoint inhibitors that can be used in the present invention include OX40 agonists. OX40 agonists that are being studied in clinical trials include PF-04518600/PF-8600 (Pfizer), an agonistic anti-OX40 antibody, in metastatic kidney cancer (NCT03092856) and advanced cancers and neoplasms (NCT02554812; NCT05082566); GSK3174998 (Merck), an agonistic anti-OX40 antibody, in Phase 1 cancer trials (NCT02528357); MEDI0562 (Medimmune/AstraZeneca), an agonistic anti-OX40 antibody, in advanced solid tumors (NCT02318394 and NCT02705482); MEDI6469, an agonistic anti-OX40 antibody (Medimmune/AstraZeneca), in patients with colorectal cancer (NCT02559024), breast cancer (NCT01862900), head and neck cancer (NCT02274155) and metastatic prostate cancer (NCT01303705); and BMS-986178 (Bristol-Myers Squibb) an agonistic anti-OX40 antibody, in advanced cancers (NCT02737475).

Checkpoint inhibitors that can be used in the present invention include CD137 (also called 4-1BB) agonists. CD137 agonists that are being studied in clinical trials include utomilumab (PF-05082566, Pfizer) an agonistic anti-CD137 antibody, in diffuse large B-cell lymphoma (NCT02951156) and in advanced cancers and neoplasms (NCT02554812 and NCT05082566); urelumab (BMS-663513, Bristol-Myers Squibb), an agonistic anti-CD137 antibody, in melanoma and skin cancer (NCT02652455) and glioblastoma and gliosarcoma (NCT02658981); and CTX-471 (Compass Therapeutics), an agonistic anti-CD137 antibody in metastatic or locally advanced malignancies (NCT03881488).

Checkpoint inhibitors that can be used in the present invention include CD27 agonists. CD27 agonists that are being studied in clinical trials include varlilumab (CDX-1127, Celldex Therapeutics) an agonistic anti-CD27 antibody, in squamous cell head and neck cancer, ovarian carcinoma, colorectal cancer, renal cell cancer, and glioblastoma (NCT02335918); lymphomas (NCT01460134); and glioma and astrocytoma (NCT02924038).

Checkpoint inhibitors that can be used in the present invention include glucocorticoid-induced tumor necrosis factor receptor (GITR) agonists. GITR agonists that are being studied in clinical trials include TRX518 (Leap Therapeutics), an agonistic anti-GITR antibody, in malignant melanoma and other malignant solid tumors (NCT01239134 and NCT02628574); GWN323 (Novartis), an agonistic anti-GITR antibody, in solid tumors and lymphoma (NCT 02740270); INCAGN01876 (Incyte/Agenus), an agonistic anti-GITR antibody, in advanced cancers (NCT02697591 and NCT03126110); MK-4166 (Merck), an agonistic anti-GITR antibody, in solid tumors (NCT02132754) and MEDI1873 (Medimmune/AstraZeneca), an agonistic hexameric GITR-ligand molecule with a human IgG1 Fc domain, in advanced solid tumors (NCT02583165).

Checkpoint inhibitors that can be used in the present invention include inducible T-cell co-stimulator (ICOS, also known as CD278) agonists. ICOS agonists that are being studied in clinical trials include MEDI-570 (Medimmune), an agonistic anti-ICOS antibody, in lymphomas (NCT02520791); GSK3359609 (Merck), an agonistic anti-ICOS antibody, in Phase 1 (NCT02723955); JTX-2011 (Jounce Therapeutics), an agonistic anti-ICOS antibody, in Phase 1 (NCT02904226).

Checkpoint inhibitors that can be used in the present invention include killer IgG-like receptor (KIR) inhibitors. KIR inhibitors that are being studied in clinical trials include lirilumab (IPH2102/BMS-986015, Innate Pharma/Bristol-Myers Squibb), an anti-KIR antibody, in leukemias (NCT01687387, NCT02399917, NCT02481297, NCT02599649), multiple myeloma (NCT02252263), and lymphoma (NCT01592370); IPH2101 (1-7F9, Innate Pharma) in myeloma (NCT01222286 and NCT01217203); and IPH4102 (Innate Pharma), an anti-KIR antibody that binds to three domains of the long cytoplasmic tail (KIR3DL2), in lymphoma (NCT02593045).

Checkpoint inhibitors that can be used in the present invention include CD47 inhibitors of interaction between CD47 and signal regulatory protein alpha (SIRPa). CD47/SIRPa inhibitors that are being studied in clinical trials include ALX-148 (Alexo Therapeutics), an antagonistic variant of (SIRPa) that binds to CD47 and prevents CD47/SIRPa-mediated signaling, in phase 1 (NCT03013218); TTI-621 (SIRPa-Fc, Trillium Therapeutics), a soluble recombinant fusion protein created by linking the N-terminal CD47-binding domain of SIRPa with the Fc domain of human IgG1, acts by binding human CD47, and preventing it from delivering its “do not eat” signal to macrophages, is in clinical trials in Phase 1 (NCT02890368 and NCT02663518); CC-90002 (Celgene), an anti-CD47 antibody, in leukemias (NCT02641002); and Hu5F9-G4 (Forty Seven, Inc.), in colorectal neoplasms and solid tumors (NCT02953782), acute myeloid leukemia (NCT02678338) and lymphoma (NCT02953509).

Checkpoint inhibitors that can be used in the present invention include CD73 inhibitors. CD73 inhibitors that are being studied in clinical trials include MEDI9447 (Medimmune), an anti-CD73 antibody, in solid tumors (NCT02503774); and BMS-986179 (Bristol-Myers Squibb), an anti-CD73 antibody, in solid tumors (NCT02754141).

Checkpoint inhibitors that can be used in the present invention include agonists of stimulator of interferon genes protein (STING, also known as transmembrane protein 173, or TMEM173). Agonists of STING that are being studied in clinical trials include MK-1454 (Merck), an agonistic synthetic cyclic dinucleotide, in lymphoma (NCT03010176); and ADU-S100 (MIW815, Aduro Biotech/Novartis), an agonistic synthetic cyclic dinucleotide, in Phase 1 (NCT02675439 and NCT03172936).

Checkpoint inhibitors that can be used in the present invention include CSF1R inhibitors. CSF1R inhibitors that are being studied in clinical trials include pexidartinib (PLX3397, Plexxikon), a CSF1R small molecule inhibitor, in colorectal cancer, pancreatic cancer, metastatic and advanced cancers (NCT02777710) and melanoma, non-small cell lung cancer, squamous cell head and neck cancer, gastrointestinal stromal tumor (GIST) and ovarian cancer (NCT02452424); and IMC-CS4 (LY3022855, Lilly), an anti-CSF-1R antibody, in pancreatic cancer (NCT03153410), melanoma (NCT03101254), and solid tumors (NCT02718911); and BLZ945 (4-[2((1R,2R)-2-hydroxycyclohexylamino)-benzothiazol-6-yloxyl]-pyridine-2-carboxylic acid methylamide, Novartis), an orally available inhibitor of CSF1R, in advanced solid tumors (NCT02829723).

Checkpoint inhibitors that can be used in the present invention include NKG2A receptor inhibitors. NKG2A receptor inhibitors that are being studied in clinical trials include monalizumab (IPH2201, Innate Pharma), an anti-NKG2A antibody, in head and neck neoplasms (NCT02643550) and chronic lymphocytic leukemia (NCT02557516).

In some embodiments, the immune checkpoint inhibitor is selected from nivolumab, pembrolizumab, ipilimumab, avelumab, durvalumab, atezolizumab, or pidilizumab.

### EXEMPLIFICATION

The following Examples illustrate the invention described above; they are not, however, intended to limit the scope of the invention in any way. The beneficial effects of the pharmaceutical compounds, combinations, and compositions of the present invention can also be determined by other test models known as such to the person skilled in the pertinent art.

### Example 1: Transcriptional Profiling Study

Profiling Study with BCY12491

For the transcriptional and immunohistochemical (IHC) analyses, 6-8 week old female huCD137-C57B/6J mice (Biocytogen) mice were implanted subcutaneously with 1×10⁶ MC38 cells. Mice were randomized into treatment groups when average tumor volumes reached around 240 mm³ and were treated intravenously with vehicle (25 mM histidine, 10% sucrose, pH7), 15 mg/kg BCY12491, 15 mg/kg BCY13626 (non-binding control) or intraperitoneally with 2 mg/kg anti-CD137 antibody urelumab. Treatments were given Q3D for three doses, tumor growth was monitored by caliper measurements and tumor tissues were harvested 1 hour after the last dose on Day 6. Part of the tumor tissue was used for RNA isolation for transcriptional analysis and a part of the tumor tissue was used for formalin-fixed paraffin embedded (FFPE) sample preparation for IHC analysis. RNA was isolated from tumor tissues using RNAeasy kit (Qiagen) and transcriptional analysis was performed using nCounter Mouse PanCancer IO 360 panel (Nanostring) from 100 ng RNA/tumor. Data were analyzed using the nSolver Analysis Software with advanced analysis probe set ns_mm_io_360_v1.0 (Nanostring). CD8+ tumor infiltrating cells were stained in FFPE tissue sections using anti-mouse CD8 antibody (Abcam, #ab217344) and Ventana Discovery OmniMap anti Rabbit-HRP Kit (Ventana #760 4310).

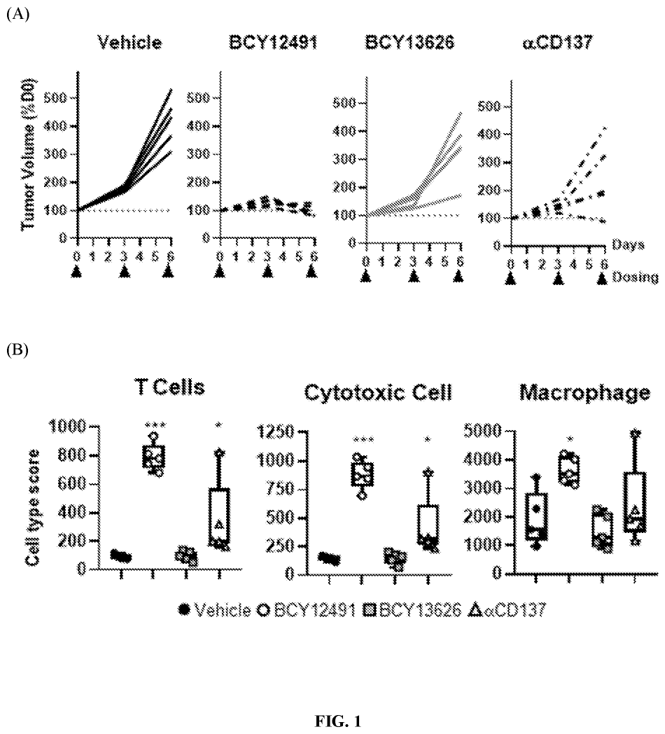

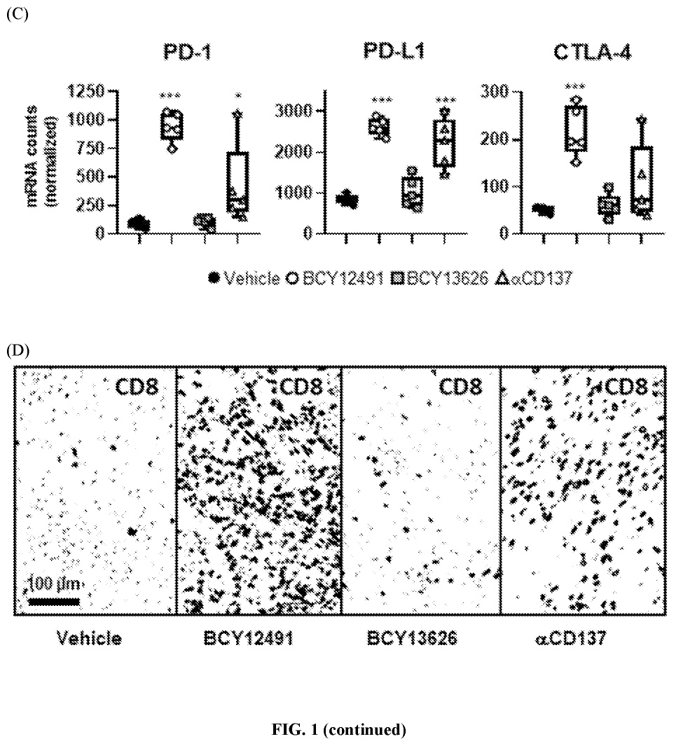

The data are shown in FIG. 1.

Findings: Transcriptional analysis revealed a significant increase in immune cell scores such as cytotoxic cell score, T cell score and macrophage cell score in tumor tissue upon EPhA2 heterotandem bicyclic peptide complex BCY12491 treatment when compared to tumors from vehicle treated mice. The anti-CD137 antibody treatment also increased significantly the cytotoxic cell score and T cell score in tumor tissue, although to lesser extent than BCY12491. No changes were observed in immune cell scores in tumor tissues from non-binding control (BCY13626) treated animals. IHC analysis for CD8+ cells in the tumor tissues demonstrated an intense infiltration of CD8+ cells in the tumors from BCY12491 treated mice when compared to tumors from vehicle or non-binder BCY13626 treated mice. Some increase of CD8+ cell infiltration was also observed in tumors from anti-CD137 antibody treated mice. These changes in immune cell scores and CD8+ cells in tumor tissue indicate that agonism of CD137 in tumor tissue by an EphA2/CD137 heterotandem bicyclic peptide complex BCY12491 leads to a significant modulation (increase) of the tumor infiltrating immune cells and immune response.

Study with BT7480

For the transcriptional and immunohistochemical (IHC) analyses, 6-8 week old female huCD137-C57B/6J mice (Biocytogen) mice were implanted subcutaneously with 1×10⁶ MC38 #13 (MC38 cells engineered to express Nectin-4) cells. Mice were randomized into treatment groups when average tumor volumes reached around 255 mm³ to receive vehicle, BT7480 (BCY00011863), non-binder BCY control BCY00012797 (BCY12797) or αCD137 antibody (urelumab analogue). BT7480 and its non-binding control were dosed intravenously at 5 mg/kg (in 25 mM histidine HCl, 10% sucrose, pH7; Vehicle) at 0 h and 24 h and urelumab analogue was dosed intraperitoneally at 2 mg/kg in PBS BIW (0 h, 72 h) dose and schedule. Tumors from BT7480-treated mice were harvested at 24 h (after the 0 h dose), 48 h (24 h after the last of 0 h and 24 h dose), 96 h (72 h after the last of 0 h and 24 h dose) and 144 h (120 h after the last of 0 h and 24 h dose). Tumors from αCD137-treated mice were harvested at 144 h after treatment initiation. Tumors from vehicle treated mice were harvested 24 h after the 0 h dose and at 144 h (120 h after the last of 0 h and 24 h dose). RNA was isolated from tumor tissues using RNAeasy kit (Qiagen) and transcriptional analysis was performed using nCounter Mouse PanCancer IO 360 panel (Nanostring) from 100 ng RNA/tumor. Data were analyzed using the nSolver Analysis Software with advanced analysis probe set ns_mm_io_360_v1.0 (Nanostring).

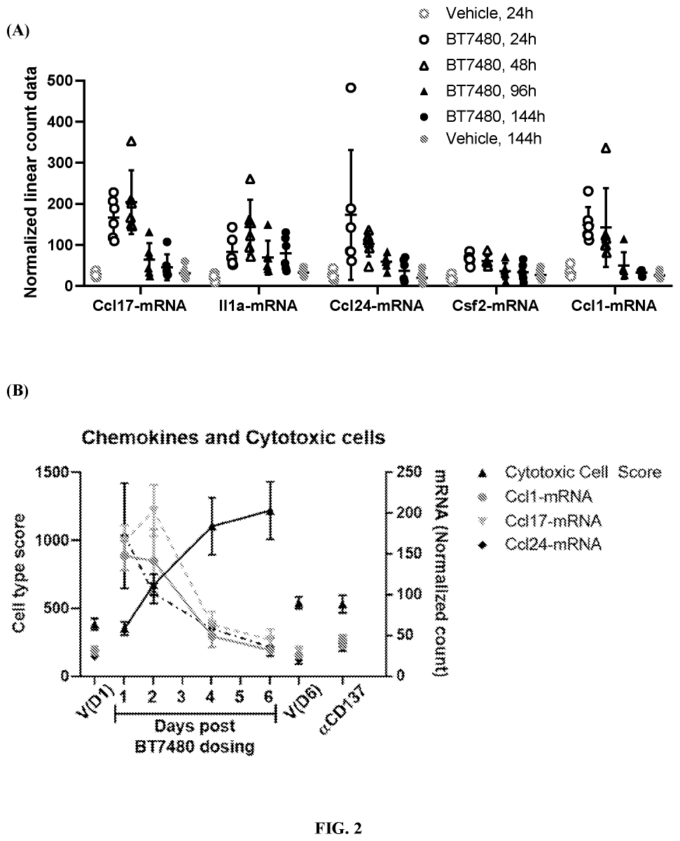

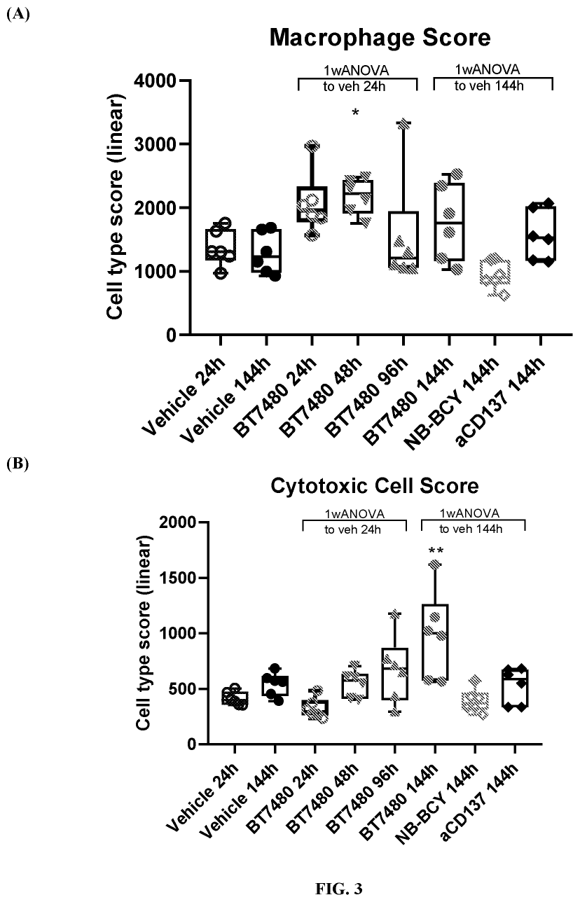

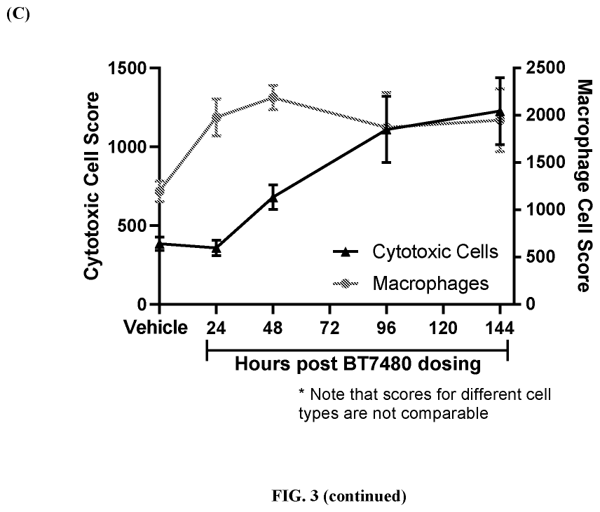

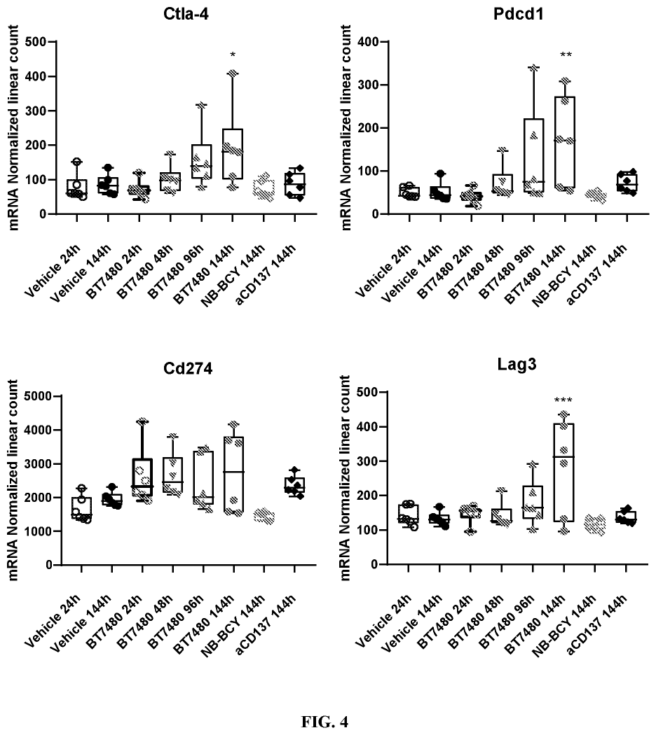

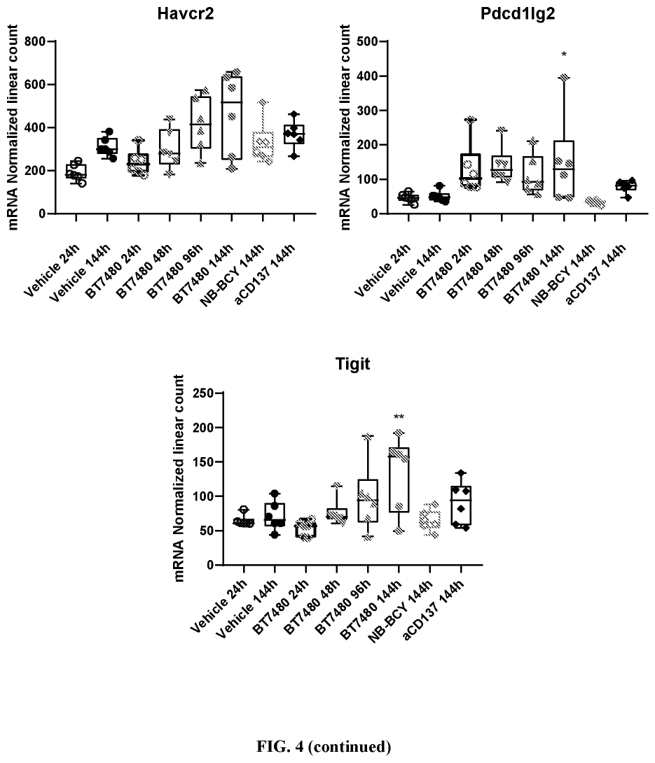

The data are shown in FIGS. 2-4.

Findings: Transcriptional analysis revealed a significant early (24 hour timepoint) increase in mRNA for several T cell chemotactic chemokines/cytokines such as Ccl1, Ccl17 and Ccl24 among others that are considered to be secreted by the myeloid cells leading to recruitment of T cells in the site of chemokine secretion. Transcriptional analysis also revealed a significant increase in immune cell scores such as cytotoxic cell score and macrophage cell score in tumor tissue upon BT7480 treatment when compared to tumors from vehicle treated mice. Macrophage Cell Score started increasing at 24 h after BT7480 administration reaching a significant increase from 24 h vehicle readout by 48 h. Cytotoxic Cell score on the other hand started increasing by 48 hours after treatment initiation and increased until 144 h when the cytotoxic cell score was significantly increased compare to the vehicle treated tumors at 144 h. Overlaying the cytotoxic cell score and the normalized mRNA counts for Ccl1, Ccl17 and Ccl24 in response to BT7480 demonstrates how the increase in the Ccl1, Ccl17 and Ccl24 transcription precedes the increase in cytotoxic cell scores.

Overlaying the macrophage and cytotoxic cell scores in response to BT7480 demonstrates how the macrophage cell score increase precedes the increase in cytotoxic cell scores.

Transcriptional analysis revealed a trend to increase or significant increase in mRNAs for several different immune checkpoints including CTLA-4 (Ctla4), PD-1 (Pdcd1), PD-L1 (Cd274), LAG3 (Lag3), TIM3 (Havcr2), PD-L2 (Pdcd1lg2) and TIGIT (Tigit) supporting the concept of BT7480 combinations with checkpoint inhibitors.

### Example 2: Efficacy Study with BCY12491 and Pembrolizumab Combination

For tumor growth analysis, 6-8 week old female huCD137/huPD-1-C57B/6J mice (Biocytogen) mice were implanted subcutaneously with 1×10⁶ MC38 cells. Mice were randomized into treatment groups when average tumor volumes reached around 92 mm³ and were treated intravenously with vehicle (25 mM histidine, 10% sucrose, pH7), 5 mg/kg BCY12491 (0, 24 h) or intraperitoneally with 3 mg/kg anti-PD-1 antibody Pembrolizumab or a combination of BCY12491 and Pembrolizumab. Combination treatments were given with three different dosing schedules: BCY12491 and Pembrolizumab treatment initiating at the same time on day 0, BCY12491 treatment initiating on day 1 and Pembrolizumab treatment initiating on day 5, or Pembrolizumab treatment initiating of day 0 and BCY12491 treatment initiating on day 5. Treatments were given weekly for four doses and tumor growth was monitored by caliper measurements.

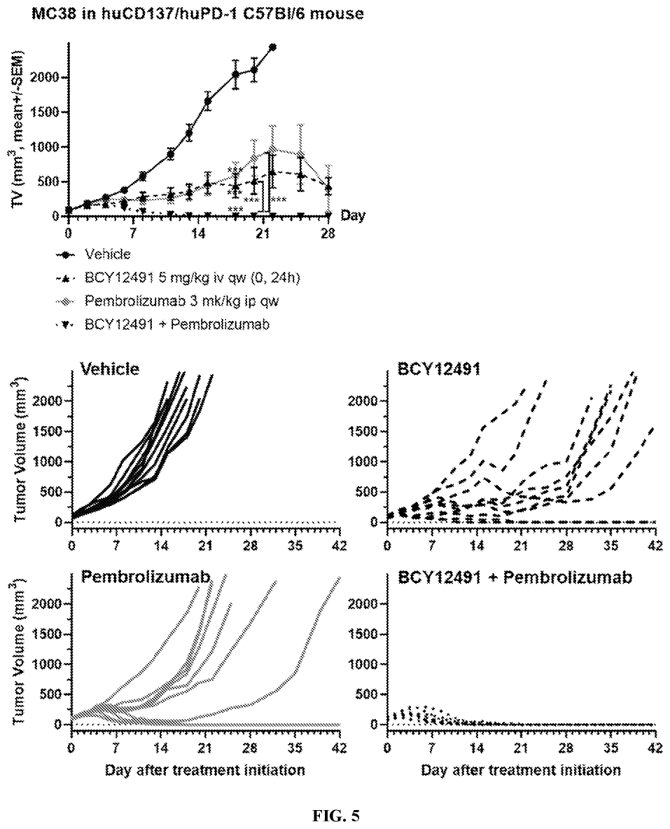

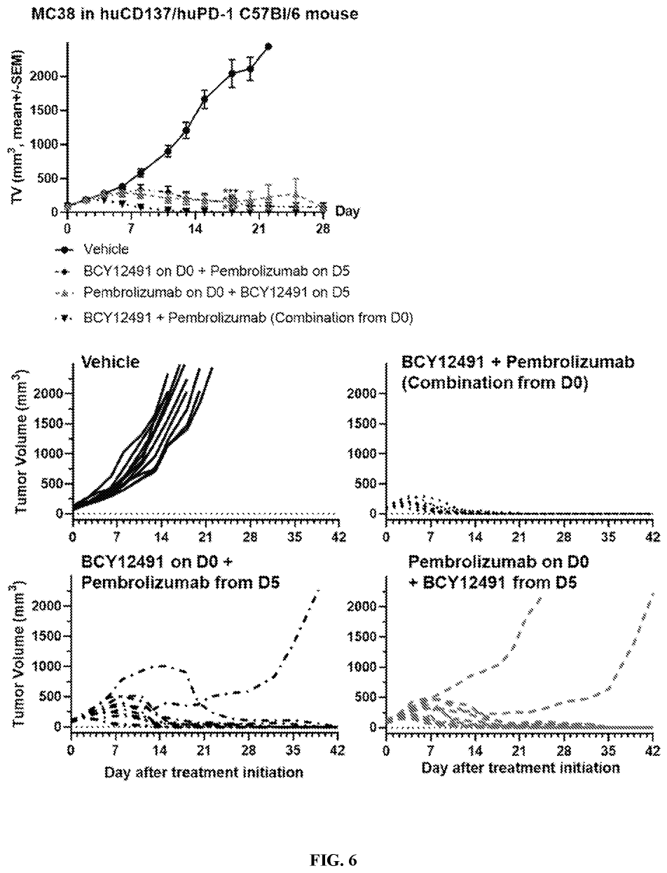

The data are shown in FIGS. 5 and 6.

Findings: Both BCY12491 and Pembrolizumab monotherapies and their combination showed significant anti-tumor activity when compared to vehicle control (all ***p<0.0001, mixed effects analysis with Dunnett's post test on D18 comparing treatments to vehicle). Furthermore, the combination treatment was more efficacious than either one of the monotherapies (***p<0.0001, mixed effects analysis with Dunnett's post test on D20 comparing combination to monotherapies) leading to complete responses in all treated animals by day 22. In contrast, these treatment regimens and schedules lead to 2/10 complete responses in BCY12491 monotherapy treatment cohort and 3/10 complete responses in Pembrolizumab monotherapy treatment cohort. The alternate sequencing of the combination of BCY12491 and Pembrolizumab (BCY12491 treatment initiating on day 0 and Pembrolizumab treatment initiating on day 5, or vice versa) also lead to significant anti-tumor activity (both ***p<0.0001, mixed effects analysis with Dunnett's post test on D18 comparing treatments to vehicle), both schedules leading to 9/10 complete responses (BCY12491 treatment initiating on day 0 and Pembrolizumab treatment initiating on day 5) and 8/10 complete responses (Pembrolizumab treatment initiating on day 0 and BCY12491 treatment initiating on day 5) in treated mice by day 42.

### Example 3: Efficacy Study with BCY11864 and Anti-PD-1 Combination

For tumor growth analysis, 6-8 week old female Balb/c-huCD137− mice (Gempharmatech) were implanted subcutaneously with 3×10+e5 CT26 #7 cells (CT26 cells engineered to overexpress Nectin-4). Mice were randomized into treatment groups when average tumor volumes reached around 80 mm³ and were treated intravenously with vehicle (25 mM histidine, 10% sucrose, pH7), 10 mg/kg BCY11864 (0, 24 h) or intraperitoneally with 10 mg/kg anti-PD-1 antibody (RMP1-14) or a combination of BCY11864 and anti-PD-1 antibody. Treatments were given weekly and tumor growth was monitored by caliper measurements. Animals with >2000 mm³ tumors were sacrificed as they had reached the Humane Endpoint. Study was terminated on day 66 after treatment initiation at which point only 2 animals (both in the combination treatment arm) remained in the study (one complete responder and one with a tumor that was still regressing in size).

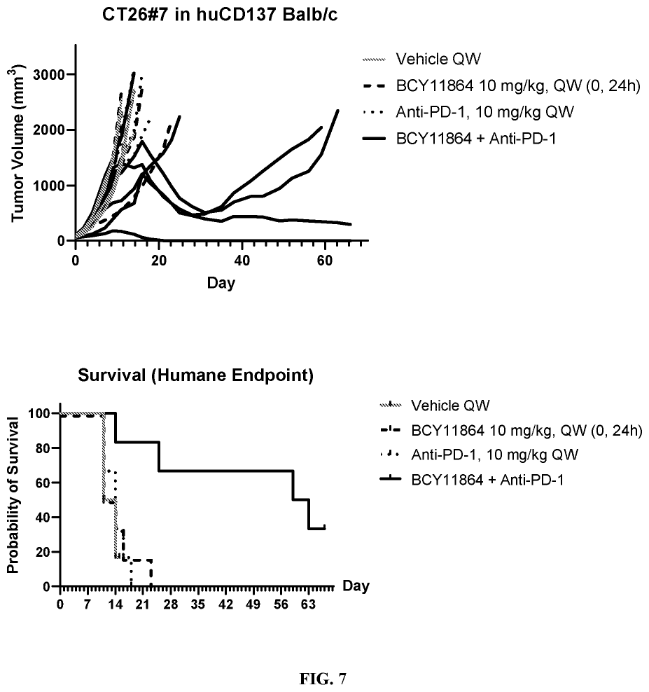

The data are shown in FIG. 7.

Findings: Addition of BCY11864 to anti-PD-1 monotherapy significantly (p=0.004, Mantel-Cox Log-rank test comparing anti-PD-1 and anti-PD-1+BCY11864 combination arms) increased the survival (outcomes measured as time to reach the Humane Endpoint i.e., tumor volumes >2000 mm³) of the CT26 #7 bearing mice.

### Example 4. Efficacy Study with BT7480 in Combination with Anti-PD-1 and Anti-Ctla-4

For tumor growth analysis, 6-8 week old female C57BL/6J-huCD137− mice (Biocytogen) were implanted subcutaneously with 1×10+e6 MC38 #13 cells (MC38 cells engineered to overexpress Nectin-4). Mice were randomized into treatment groups when average tumor volumes reached around 100 mm³ and were treated intraperitoneally with vehicle (25 mM histidine, 10% sucrose, pH7), 1 mg/kg BT7480, 5 mg/kg anti-PD-1 (RMP 1-14), 5 mg/kg anti-Ctla-4 (9H10) or BT7480/anti-PD-1 and BT7480/anti-Ctla-4 combinations. Treatments were given twice weekly (BIW) for 2 weeks, and tumor growth was monitored by caliper measurements until day 33 after treatment initiation. Animals with >2000 mm³ tumors were sacrificed as they had reached the Humane Endpoint.

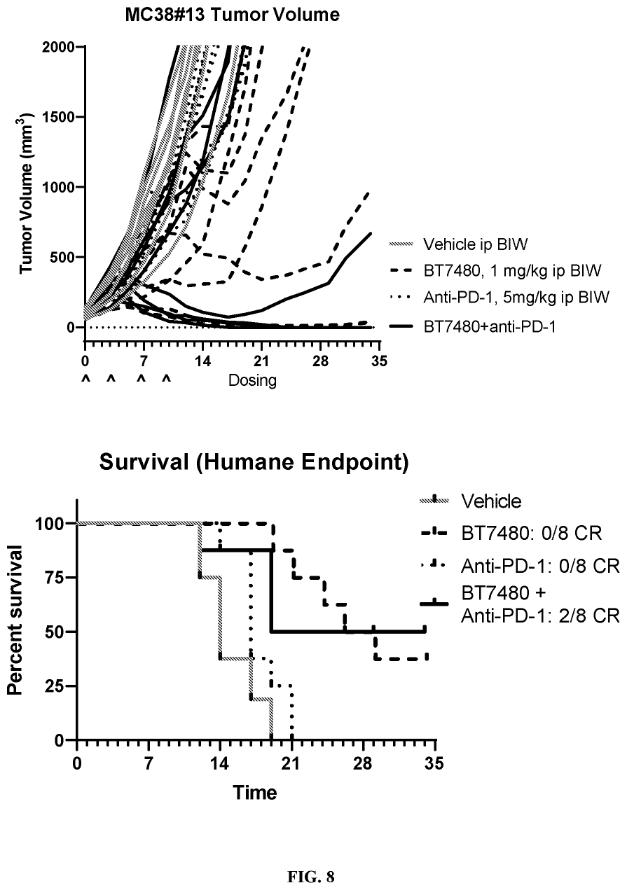

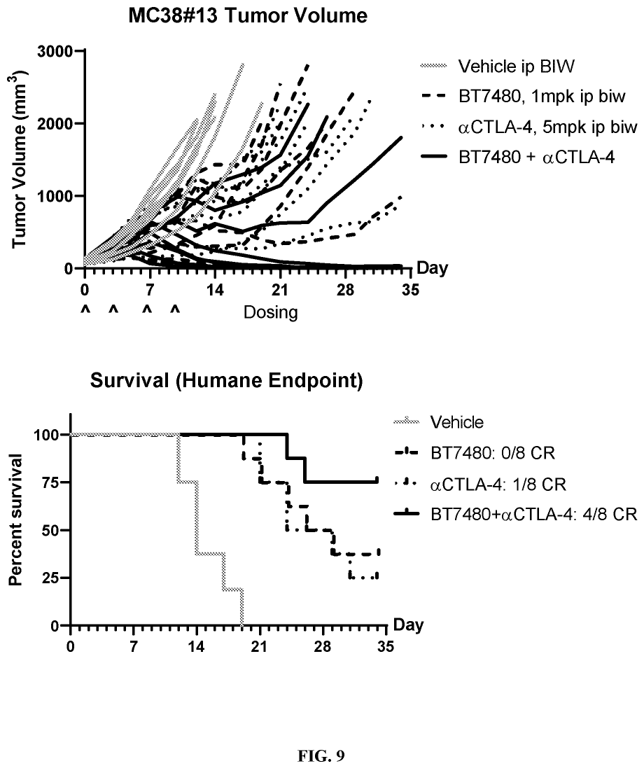

The data are shown in FIGS. 8 and 9.

Findings: Addition of BT7480 to anti-PD-1 monotherapy increased the rate of complete responses (CRs) from 0/8 (in BT7480 and anti-PD-1 monotherapy arms) to 2/8 in the BT7480/anti-PD-1 combination treatment arm. Addition of BT7480 to anti-Ctla-4 monotherapy increased the rate of complete responses (CR) from 0/8 or 1/8 (in BT7480 and anti-Ctla-4 monotherapy arms, respectively) to 4/8 in the BT7480/anti-Ctla-4 combination treatment arm by day 33 after treatment initiation. Furthermore, addition of BT7480 to anti-CTLA-4 monotherapy significantly (p=0.0499, Mantel-Cox Log-rank test comparing anti-Ctla-4 and anti-Ctla-4+BT7480 combination arms) increased the survival (outcomes measured as time to reach the Humane Endpoint i.e., tumor volumes >2000 mm³) of MC38 #13 bearing mice.

### Example 5. Transcriptional Profiling Study with BT7455

For the transcriptional profiling analyses of the effects of BT7455 in the immune tumor microenvironment, 6-8 week old female huCD137-C57B/6J mice (Biocytogen) mice were implanted subcutaneously with 1×10+E6 MC38 cells. Mice were randomized into treatment groups when average tumor volumes reached around 350 mm³ to receive vehicle, BT7455, αCD137 antibody (urelumab analogue) or αPD-1 antibody. BT7455 was dosed intravenously at 8 mg/kg (in 25 mM histidine HCl, 10% sucrose, pH7; Vehicle) at 0 h and 24 h and urelumab analogue and αPD-1 antibody were dosed intraperitoneally at 2 mg/kg (urelumab analogue) or 10 mg/kg (αPD-1 antibody) in PBS at 0 h. Tumors from vehicle, BT7455, urelumab analogue and αPD-1 antibody-treated mice were harvested at 24 h, 48 h and 144 h after treatment initiation. RNA was isolated from tumor tissues using RNAeasy kit (Qiagen) and transcriptional analysis was performed using nCounter Mouse PanCancer IO 360 panel (Nanostring) from 100 ng RNA/tumor. Data were analyzed using the nSolver Analysis Software with advanced analysis probe set ns_mm_io_360 v1.0 (Nanostring).

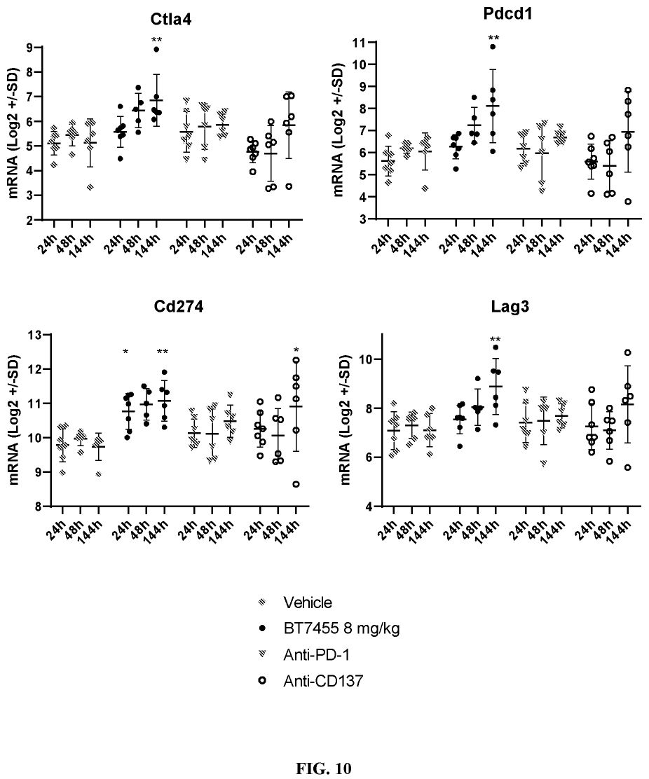

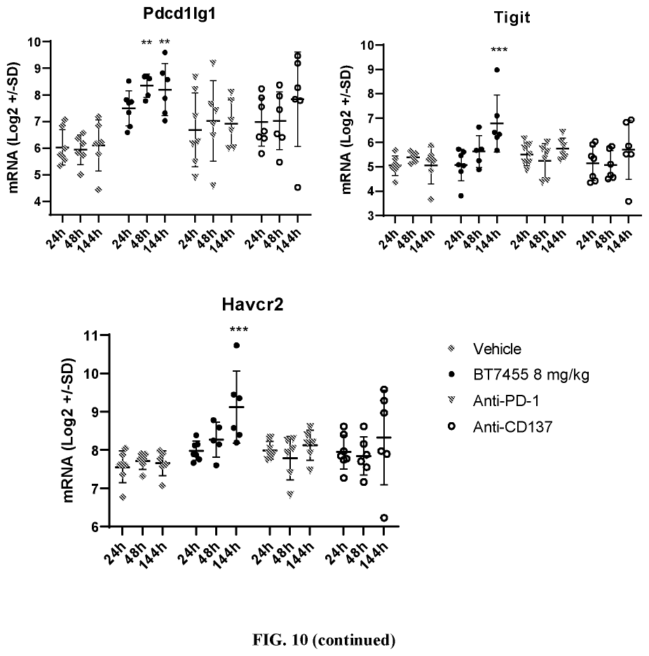

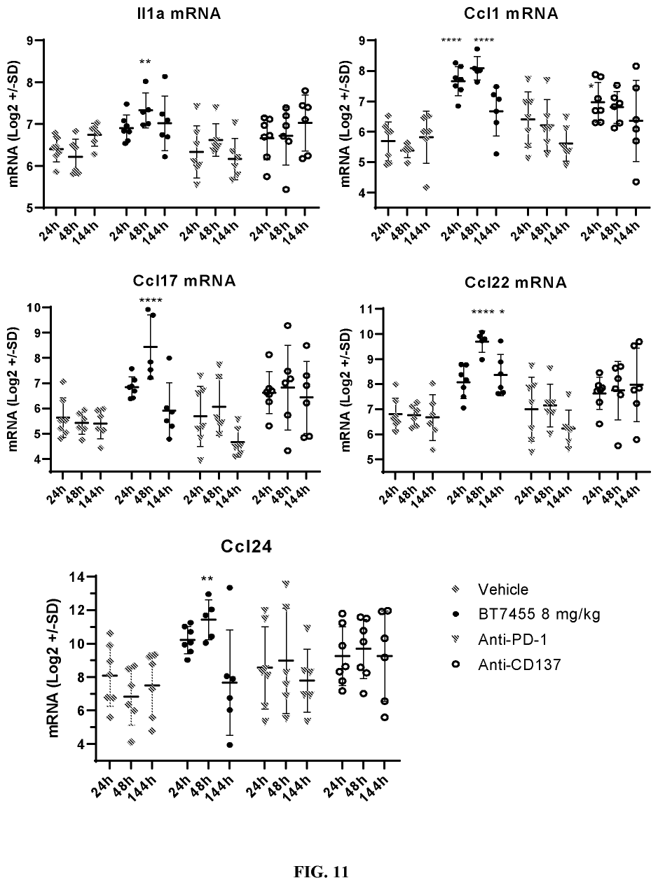

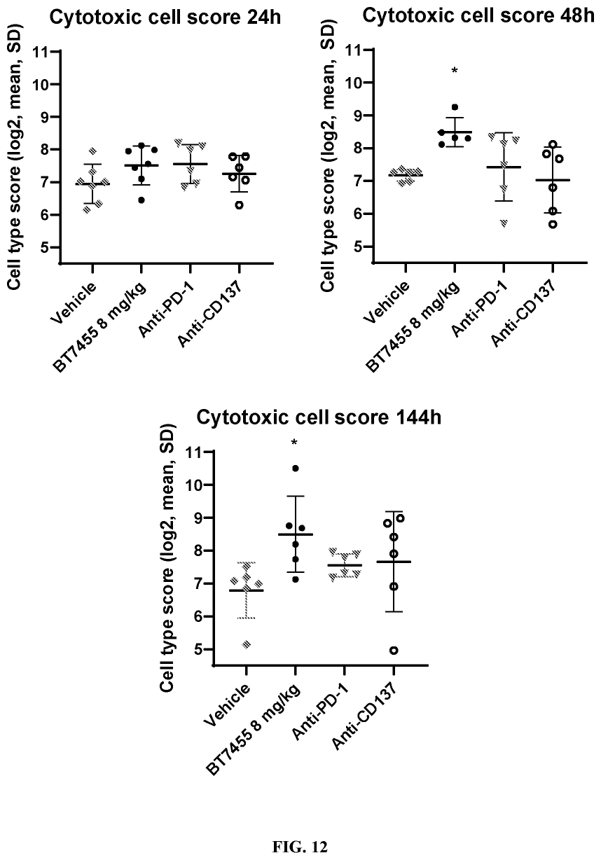

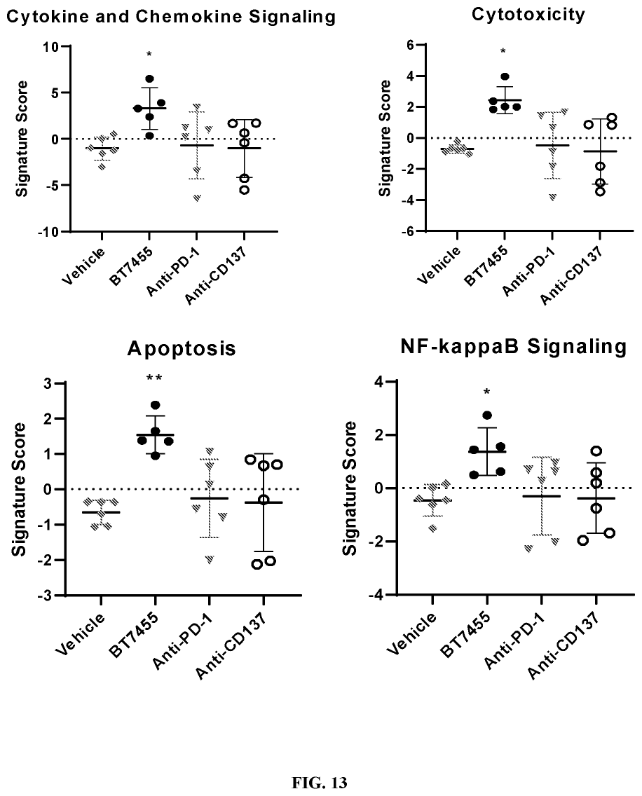

The data are shown in FIGS. 10-13.

Findings: Transcriptional analysis revealed a significant increase after BT7455 treatment in mRNAs for several different immune checkpoints including CTLA-4 (Ctla4), PD-1 (Pdcd1), PD-L1 (Cd274), LAG3 (Lag3), TIM3 (Havcr2), PD-L2 (Pdcd1lg2) and TIGIT (Tigit) supporting the concept of BT7455 combinations with checkpoint inhibitors. Transcriptional analysis also revealed a significant early (24-48 hour timepoint) increase in mRNA for several T cell chemotactic chemokines/cytokines such as Ccl1, Ccl17 and Ccl24 among others that are considered to be secreted by the myeloid cells leading to recruitment of T cells in the site of chemokine secretion. Transcriptional analysis also revealed a significant increase in immune cell scores such as cytotoxic cell score in tumor tissue upon BT7455 treatment when compared to tumors from vehicle or anti-PD-1 or anti-CD137 treated mice. BT7455 treatment elicited significant early (48 hours) modulation of several gene sets, including gene sets associated with cytokine and chemokine signaling, cytotoxicity, apoptosis and NK-kappaB signaling gene sets.

1. A method of treating a cancer in a patient, comprising administering to said patient a therapeutically effective amount of a heterotandem bicyclic peptide complex, or a pharmaceutically acceptable salt thereof, and an immuno-oncology agent, wherein the heterotandem bicyclic peptide complex comprises:
(a) a first peptide ligand which binds to a component present on a cancer cell, wherein the component present on the cancer cell is Nectin-4, and the first peptide ligand comprises an Nectin-4 binding bicyclic peptide ligand; conjugated via a linker to
(b) one or more CD137 binding bicyclic peptide ligands;
wherein each of said peptide ligands comprise a polypeptide comprising at least three reactive groups, separated by at least two loop sequences, and a molecular scaffold which forms covalent bonds with the reactive groups of the polypeptide such that at least two polypeptide loops are formed on the molecular scaffold.

2. The method of claim 1, wherein the reactive groups are cysteine residues.

3. The method of claim 1, wherein the heterotandem bicyclic peptide complex comprises
two or more CD137 binding bicyclic peptide ligands.

4. (canceled)

5. The method of claim 1, wherein the one or more CD137 binding bicyclic peptide ligands each comprise an amino acid sequence independently selected from:
(SEQ ID NO: 5)
CᵢᵢEEGQYCᵢᵢFADPY[Nle]Cᵢᵢᵢ;
(SEQ ID NO: 6)
Cᵢ[tBuAla]PE[D-Ala]PYCᵢᵢFADPY[Nle]Cᵢᵢᵢ;
(SEQ ID NO: 7)
CᵢᵢEEGQYCᵢᵢF[D-Ala]DPY[Nle]Cᵢᵢᵢ;
(SEQ ID NO: 8)
Cᵢ[tBuAla]PK[D-Ala]PYCᵢᵢFADPY[Nle]Cᵢᵢᵢ;
(SEQ ID NO: 9)
Cᵢ[tBuAla]PE[D-Lys]PYCᵢᵢFADPY[Nle]Cᵢᵢᵢ;
(SEQ ID NO: 10)
Cᵢ[tBuAla]P[K(PYA)][D-Ala]PYCᵢᵢFADPY[Nle]Cᵢᵢᵢ;
(SEQ ID NO: 11)
Cᵢ[tBuAla]PE[D-Lys(PYA)]PYCᵢᵢFADPY[Nle]Cᵢᵢᵢ;
(SEQ ID NO: 11)-A
(herein referred to as BCY14601);
(SEQ ID NO: 12)
CᵢᵢEE[D-Lys(PYA)]QYCᵢᵢFADPY(Nle)Cᵢᵢᵢ;
(SEQ ID NO: 60)
Cᵢ[tBuAla]PE[dK]PYCᵢᵢFADPY[Nle]Cᵢᵢᵢ;
(SEQ ID NO: 61)
CᵢᵢEE[dK(PYA)]QYCᵢᵢFADPY[Nle]Cᵢᵢᵢ;
(SEQ ID NO: 62)
Cᵢ[tBuAla]EE(dk)PYCᵢᵢFADPY[Nle]Cᵢᵢᵢ;
(SEQ ID NO: 63)
Cᵢ[tBuAla]PE[dK(PYA)]PYCᵢᵢFADPY[Nle]Cᵢᵢᵢ;
(SEQ ID NO: 64)
Cᵢ[tBuAla]EE[dK(PYA)]PYCᵢᵢFADPY[Nle]Cᵢᵢᵢ;
(SEQ ID NO: 65)
Cᵢ[tBuAla]PE[dK(PYA)]PYCᵢᵢFANPY[Nle]Cᵢᵢᵢ;
(SEQ ID NO: 66)
Cᵢ[tBuAla]PE[dK(PYA)]PYCᵢᵢFAEPY[Nle]Cᵢᵢᵢ;
(SEQ ID NO: 67)
Cᵢ[tBuAla]PE[dK(PYA)]PYCᵢᵢFA[Aad]PY[Nle]Cᵢᵢᵢ;
(SEQ ID NO: 68)
Cᵢ[tBuAla]PE[dK(PYA)]PYCᵢᵢFAQPY[Nle]Cᵢᵢᵢ;
(SEQ ID NO: 69)
Cᵢ[tBuAla]PE[dK(PYA)]PYCᵢᵢFADPY[Nle][Cysam]ᵢᵢᵢ;
(SEQ ID NO: 70; herein referred to as BCY12353)
[MerPro]ᵢ[tBuAla]PE[dK(PYA)]PYCᵢᵢFADPY[Nle]Cᵢᵢᵢ;
(SEQ ID NO: 71; herein referred to as BCY12354)
[MerPro]ᵢ[tBuAla]PE[dK(PYA)]PYCᵢᵢFA
DPY[Nle][Cysam]ᵢᵢᵢ;
(SEQ ID NO: 72)
Cᵢ[tBuAla]PE[dK(PYA)]PYCᵢᵢFADPY[Nle]Cᵢᵢᵢ;
(SEQ ID NO: 73)
Cᵢ[tBuAla]PE[dK(PYA)]PYCᵢᵢFADPY[Nle]Cᵢᵢᵢ;
(SEQ ID NO: 74; herein referred to as BCY12372)
Cᵢ[tBuAla]PE[dK(PYA)]PYCᵢᵢFADPY[Nle]Cᵢᵢᵢ;
(SEQ ID NO: 75)
Cᵢ[tBuAla]PE[dK(PYA)]PYCᵢᵢFAD[NMeAla]Y[Nle]Cᵢᵢᵢ;
(SEQ ID NO: 76)
Cᵢ[tBuAla]PE[dK(PYA)]PYCᵢᵢFAD[NMeDAla]Y[Nle]Cᵢᵢᵢ;
(SEQ ID NO: 77)
Cᵢ[tBuAla]P[K(PYA)][dA]PYCᵢᵢFADPY[Nle]Cᵢᵢᵢ;
(SEQ ID NO: 78)
Cᵢ[tBuAla]PE[dK(PYA)]PYCᵢᵢFADPY[Nle]Cᵢᵢᵢ;
(SEQ ID NO: 79)
Cᵢ[tBuAla]PE[dK(Me,PYA)]PYCᵢᵢFADPY[Nle]Cᵢᵢᵢ;
(SEQ ID NO: 80)
Cᵢ[tBuAla]PE[dK(Me,PYA)]PYCᵢᵢFADPY[Nle]Cᵢᵢᵢ;
and
(SEQ ID NO: 81; herein referred to as BCY13137)
[MerPro]ᵢ[tBuAla]EE[dK]PYCᵢᵢFADPY[Nle]Cᵢᵢᵢ;
wherein [MerPro]ᵢ, Cᵢ, Cᵢᵢ, Cᵢᵢᵢ and [Cysam]ᵢᵢᵢ represent first (i), second (ii) and third (iii) reactive groups which are selected from cysteine, MerPro and Cysam, Ne represents norleucine, tBuAla represents t-butyl-alanine, PYA represents 4-pentynoic acid, Aad represents alpha-L-aminoadipic acid, MerPro represents 3-mercaptopropionic acid and Cysam represents cysteamine, NMeAla represents N-methyl-alanine, or a pharmaceutically acceptable salt thereof.

6. The method of claim 1, wherein the one or more CD137 binding bicyclic peptide ligands each comprise an amino acid sequence which is:
(SEQ ID NO: 11)
Cᵢ[tBuAla]PE[D-Lys(PYA)]PYCᵢᵢFADPY[Nle]Cᵢᵢᵢ;
wherein Cᵢ, Cᵢᵢ and Cᵢᵢᵢ represent first, second and third cysteine residues, respectively, tBuAla represents t-butyl-alanine, PYA represents 4-pentynoic acid, Nle represents norleucine, or a pharmaceutically acceptable salt thereof.

7. The method of claim 1, wherein the one or more CD137 binding bicyclic peptide ligands each comprise an amino acid sequence with N- and C-terminal modifications, which is independently selected from:
Ac-A-(SEQ ID NO: 5)-Dap
(herein referred to as BCY7732);
Ac-A-(SEQ ID NO: 5)-Dap(PYA)
(herein referred to as BCY7741);
Ac-(SEQ ID NO: 6)-Dap
(herein referred to as BCY9172);
Ac-(SEQ ID NO: 6)-Dap(PYA)
(herein referred to as BCY11014);
Ac-A-(SEQ ID NO: 7)-Dap
(herein referred to as BCY8045);
Ac-(SEQ ID NO: 8)-A
(herein referred to as BCY8919);
Ac-(SEQ ID NO: 9)-A
(herein referred to as BCY8920);
Ac-(SEQ ID NO: 10)-A
(herein referred to as BCY8927);
Ac-(SEQ ID NO: 11)-A
(herein referred to as BCY8928);
Ac-A-(SEQ ID NO: 12)-A
(herein referred to as BCY7744);
Ac-(SEQ ID NO: 60)-Dap(PYA)
(herein referred to as BCY11144);
Ac-A-(SEQ ID NO: 61)-K
(herein referred to as BCY11613);
Ac-(SEQ ID NO: 62)-Dap(PYA)
(herein referred to as BCY12023);
Ac-(SEQ ID NO: 63)
(herein referred to as BCY12149);
Ac-(SEQ ID NO: 64)
(herein referred to as BCY12143);
Ac-(SEQ ID NO: 65)
(herein referred to as BCY12147);
Ac-(SEQ ID NO: 66)
(herein referred to as BCY12145);
Ac-(SEQ ID NO: 67)
(herein referred to as BCY12146);
Ac-(SEQ ID NO: 68)
(herein referred to as BCY12150);
Ac-(SEQ ID NO: 69)
(herein referred to as BCY12352);
Ac-(SEQ ID NO: 72)-[1,2-diaminoethane]
(herein referred to as BCY12358);
[Palmitic Acid]-[yGlu]-[yGlu]-(SEQ ID NO: 73)
(herein referred to as BCY12360);
Ac-(SEQ ID NO: 75)
(herein referred to as BCY12381);
Ac-(SEQ ID NO: 76)
(herein referred to as BCY12382);
Ac-(SEQ ID NO: 77)-K
(herein referred to as BCY12357);
Ac-(SEQ ID NO: 78)-[dA]
(herein referred to as BCY13095);
[Ac]-(SEQ ID NO: 78)-K
(herein referred to as BCY13389);
Ac-(SEQ ID NO: 79)-[dA]
(herein referred to as BCY13096);
and
Ac-(SEQ ID NO: 80)
(herein referred to as BCY13097);
wherein Ac represents an acetyl group, Dap represents diaminopropionic acid and PYA represents 4-pentynoic acid, or a pharmaceutically acceptable salt thereof.

8. The method of claim 1, wherein the one or more CD137 binding bicyclic peptide ligands each comprise an amino acid sequence:
Ac-(SEQ ID NO: 11)-A (herein referred to as BCY8928);
wherein Ac represents an acetyl group, or a pharmaceutically acceptable salt thereof.

9. The method of claim 8, wherein the heterotandem bicyclic peptide complex comprises two CD137 binding bicyclic peptide ligands, wherein both of said two CD137 binding bicyclic peptide ligands have the same peptide sequence which comprises Ac-(SEQ ID NO: 11)-A (herein referred to as BCY8928), or a pharmaceutically acceptable salt thereof.

10. The method of claim 1, wherein the Nectin-4 binding bicyclic peptide ligand comprises an amino acid sequence selected from:
(SEQ ID NO: 1; herein referred to as BCY8116)
CᵢP[1Nal][dD]CᵢᵢM[HArg]DWSTP[HyP]WCᵢᵢᵢ;
(SEQ ID NO: 3)
CᵢP[1Nal][dK](Sar10-(B-Ala))CᵢᵢM[HArg]
DWSTP[HyP]WCᵢᵢᵢ;
(SEQ ID NO: 4; herein referred to as BCY11414)
CᵢᵢPFGCᵢᵢM[HArg]DWSTP[HyP]WCᵢᵢᵢ;
(SEQ ID NO: 14)
CᵢP[1Nal][dK]CᵢᵢM[HArg]DWSTP[HyP]WCᵢᵢᵢ;
(SEQ ID NO: 15; herein referred to as BCY12363)
[MerPro]ᵢP[1Nal][dK]CᵢᵢM[HArg]DWSTP[HyP]WCᵢᵢᵢ;
(SEQ ID NO: 16)
CᵢᵢP[1Nal][dK]CᵢᵢM[HArg]DWSTP[HyP]W[Cysam]ᵢᵢᵢ;
(SEQ ID NO: 17; herein referred to as BCY12365);
[MerPro]ᵢP[1Nal][dK]CᵢᵢM[HArg]DWSTP[HyP]W[Cysam]ᵢᵢᵢ
(SEQ ID NO: 18)
CᵢP[1Nal][dK]CᵢᵢM[HArg]HWSTP[HyP]WCᵢᵢᵢ;
(SEQ ID NO: 19)
CᵢP[1Nal][dK]CᵢᵢM[HArg]EWSTP[HyP]WCᵢᵢᵢ;
(SEQ ID NO: 20; herein referred to as BCY12368);
CᵢP[1Nal][dE]CᵢᵢM[HArg]DWSTP[HyP]WCᵢᵢᵢ
(SEQ ID NO: 21; herein referred to as BCY12369);
CᵢP[1Nal][dA]CᵢᵢM[HArg]DWSTP[HyP]WCᵢᵢᵢ
(SEQ ID NO: 22; herein referred to as BCY12370);
CᵢP[1Nal][dE]CᵢᵢL[HArg]DWSTP[HyP]WCᵢᵢᵢ
and
(SEQ ID NO: 23; herein referred to as BCY12384);
CᵢP[1Nal][dE]CᵢᵢM[HArg]EWSTP[HyP]WCᵢᵢᵢ
wherein [MerPro]ᵢ, Cᵢ, Cᵢᵢ, Cᵢᵢᵢ and [Cysam]ᵢᵢᵢ represent first (i), second (ii) and third (iii) reactive groups which are selected from cysteine, MerPro and Cysam, 1Nal represents 1-naphthylalanine, HArg represents homoarginine, HyP represents trans-4-hydroxy-L-proline, Sar₁₀ represents 10 sarcosine units, B-Ala represents beta-alanine, MerPro represents 3-mercaptopropionic acid and Cysam represents cysteamine, or a pharmaceutically acceptable salt thereof.

11. The method of claim 10, wherein the Nectin-4 binding bicyclic peptide ligand comprises an amino acid sequence optionally comprising N-terminal modifications, wherein the amino acid sequence is selected from:
SEQ ID NO: 1 (herein referred to as BCY8116);
[PYA]-[B-Ala]-[Sar10]-(SEQ ID NO: 1)
(herein referred to as BCY8846);
[PYA]-(SEQ ID NO: 1)
(herein referred to as BCY11015);
[PYA]-[B-Ala]-(SEQ ID NO: 1)
(herein referred to as BCY11016);
[PYA]-[B-Ala]-[Sar10]-(SEQ ID NO: 2)
(herein referred to as BCY11942);
Ac-(SEQ ID NO: 3)
(herein referred to as BCY8831);
SEQ ID NO: 4 (herein referred to as BCY11414);
[PYA]-[B-Ala]-(SEQ ID NO: 14)
(herein referred to as BCY11143);
Palmitic-yGlu-yGlu-(SEQ ID NO: 14)
(herein referred to as BCY12371);
Ac-(SEQ ID NO: 14)
(herein referred to as BCY12024);
Ac-SEQ ID NO: 16)
(herein referred to as BCY12364);
Ac-SEQ ID NO: 18)
(herein referred to as BCY12366);
and
Ac-(SEQ ID NO: 19)
(herein referred to as BCY12367);
wherein PYA represents 4-pentynoic acid, B-Ala represents beta-alanine, Sar₁₀ represents 10 sarcosine units, or a pharmaceutically acceptable salt thereof.

12. The method of claim 1, wherein the Nectin-4 binding bicyclic peptide ligand comprises SEQ ID NO: 1 (herein referred to as BCY8116).

13. The method of claim 1, wherein the heterotandem bicyclic peptide complex is selected from those listed in Tables A and B, such as BCY11027, BCY11863 and BCY11864, or a pharmaceutically acceptable salt thereof.

14. A method of treating a cancer in a patient, comprising administering to said patient a therapeutically effective amount of a heterotandem bicyclic peptide complex, or a pharmaceutically acceptable salt thereof, and an immuno-oncology agent, wherein the heterotandem bicyclic peptide complex comprises:
(a) a first peptide ligand which binds to a component present on a cancer cell, wherein the component present on the cancer cell is EphA2, and the first peptide ligand comprises an EphA2 binding bicyclic peptide ligand; conjugated via a linker to
(b) one or more CD137 binding bicyclic peptide ligands;
wherein each of said peptide ligands comprise a polypeptide comprising at least three reactive groups, separated by at least two loop sequences, and a molecular scaffold which forms covalent bonds with the reactive groups of the polypeptide such that at least two polypeptide loops are formed on the molecular scaffold.

15. The method of claim 14, wherein the EphA2 binding bicyclic peptide ligand comprises an amino acid sequence selected from:
(SEQ ID NO: 24)
Cᵢ[HyP]LVNPLCᵢᵢLHP[dD]W[HArg]Cᵢᵢᵢ;
(SEQ ID NO: 25)
CᵢLWDPTPCᵢᵢANLHL[HArg]Cᵢᵢᵢ;
(SEQ ID NO: 26)
Cᵢ[HyP]LVNPLCᵢᵢL[K(PYA)]P[dD]W[HArg]Cᵢᵢᵢ;
(SEQ ID NO: 27)
Cᵢ[HyP][K(PYA)]VNPLCᵢᵢLHP[dD]W[HArg]Cᵢᵢᵢ;
(SEQ ID NO: 28)
Cᵢ[HyP]LVNPLCᵢ[K(PYA)]HP[dD]W[HArg]Cᵢᵢᵢ;
(SEQ ID NO: 29)
Cᵢ[HyP]LVNPLCᵢᵢLKP[dD]W[HArg]Cᵢᵢᵢ;
(SEQ ID NO: 30)
Cᵢ[HyP]KVNPLCᵢᵢLHP[dD]W[HArg]Cᵢᵢᵢ;
(SEQ ID NO: 31)
Cᵢ[HyP]LVNPLCᵢᵢKHP[dD]W[HArg]Cᵢᵢᵢ;
(SEQ ID NO: 32)
Cᵢ[HyP]LVNPLCᵢᵢLHP[dE]W[HArg]Cᵢᵢᵢ;
(SEQ ID NO: 33)
Cᵢ[HyP]LVNPLCᵢᵢLEP[dD]W[HArg]Cᵢᵢᵢ;
(SEQ ID NO: 34)
Cᵢ[HyP]LVNPLCᵢᵢLHP[dD]WTCᵢᵢᵢ;
(SEQ ID NO: 35)
Cᵢ[HyP]LVNPLCᵢᵢLEP[dD]WTCᵢᵢᵢ;
(SEQ ID NO: 36)
Cᵢ[HyP]LVNPLCᵢᵢLEP[dA]WTCᵢᵢᵢ;
(SEQ ID NO: 37; herein referred to as BCY12860)
Cᵢ[HyP]LVNPLCᵢᵢL[3,3-DPA]P[dD]WTCᵢᵢᵢ;
(SEQ ID NO: 38)
Cᵢ[HyP][Cba]VNPLCᵢᵢLHP[dD]W[HArg]Cᵢᵢᵢ;
(SEQ ID NO: 39)
Cᵢ[HyP][Cba]VNPLCᵢᵢLEP[dD]WTCᵢᵢᵢ;
(SEQ ID NO: 40)
Cᵢ[HyP][Cba]VNPLCᵢᵢL[3,3-DPA]P[dD]WTCᵢᵢᵢ;
(SEQ ID NO: 41)
Cᵢ[HyP]LVNPLCᵢᵢL[3,3-DPA]P[dD]W[HArg]Cᵢᵢᵢ;
(SEQ ID NO: 42)
Cᵢ[HyP]LVNPLCᵢᵢLHP[d]Nal]W[HArg]Cᵢᵢᵢ;
(SEQ ID NO: 43)
Cᵢ[HyP]LVNPLCᵢᵢL[1Nal]P[dD]W[HArg]Cᵢᵢᵢ;
(SEQ ID NO: 44)
Cᵢ[HyP]LVNPLCᵢᵢLEP[d1Nal]WTCᵢᵢᵢ;
(SEQ ID NO: 45; herein referred to as BCY13119)
Cᵢ[HyP]LVNPLCᵢᵢL[1Nal]P[dD]WTCᵢᵢᵢ;
(SEQ ID NO: 46)
Cᵢ[HyP][Cba]VNPLCᵢᵢLEP[dA]WTCᵢᵢᵢ;
(SEQ ID NO: 47)
Cᵢ[HyP][hGlu]VNPLCᵢᵢLHP[dD]W[HArg]Cᵢᵢᵢ;
(SEQ ID NO: 48)
Cᵢ[HyP]LVNPLCᵢᵢ[hGlu]HP[dD]W[HArg]Cᵢᵢᵢ;
(SEQ ID NO: 49)
Cᵢ[HyP]LVNPLCᵢᵢL[hGlu]P[dD]W[HArg]Cᵢᵢᵢ;
(SEQ ID NO: 50)
Cᵢ[HyP]LVNPLCᵢᵢLHP[dNle]W[HArg]Cᵢᵢᵢ;
(SEQ ID NO: 51)
Cᵢ[HyP]LVNPLCᵢᵢL[Nle]P[dD]W[HArg]Cᵢᵢᵢ;
(SEQ ID NO: 154)
[MerPro]i[HyP]LVNPLCᵢᵢL[3,3-DPA]P[dD]WTCᵢᵢᵢ;
(SEQ ID NO: 155)
Cᵢ[HyP]LVNPLCᵢᵢLHP[dD]W[HArg][Cysam]ᵢᵢᵢ;
(SEQ ID NO: 156)
Cᵢ[HyP]LVNPLCᵢᵢL[His3Me]P[dD]W[HArg]Cᵢᵢᵢ;
(SEQ ID NO: 157)
Cᵢ[HyP]LVNPLCᵢᵢL[His1Me]P[dD]W[HArg]Cᵢᵢᵢ;
(SEQ ID NO: 158)
Cᵢ[HyP]LVNPLCᵢᵢL[4ThiAz]P[dD]W[HArg]Cᵢᵢᵢ;
(SEQ ID NO: 159)
Cᵢ[HyP]LVNPLCᵢᵢLFP[dD]W[HArg]Cᵢᵢᵢ;
(SEQ ID NO: 160)
Cᵢ[HyP]L VNPLCᵢᵢL[Thi]P[dD]W[HArg]Cᵢᵢᵢ;
(SEQ ID NO: 161)
Cᵢ[HyP]LVNPLCᵢᵢL[3Thi]P[dD]W[HArg]Cᵢᵢᵢ;
(SEQ ID NO: 162)
Cᵢ[HyP]LVNPLCᵢᵢLNP[dD]W[HArg]Cᵢᵢᵢ;
(SEQ ID NO: 163)
Cᵢ[HyP]LVNPLCᵢᵢLQP[dD]W[HArg]Cᵢᵢᵢ;
and
(SEQ ID NO: 164)
Cᵢ[HyP]LVNPLCᵢᵢL[K(PYA-(Palmitoyl-Glu-
LysN3)]P[dD]W[HArg]Cᵢᵢᵢ;
wherein [MerPro]ᵢ, Cᵢ, Cᵢᵢ, Cᵢᵢᵢ and [Cysam]ᵢᵢᵢ represent first (i), second (ii) and third (iii) reactive groups which are selected from cysteine, MerPro and Cysam, HyP represents trans-4-hydroxy-L-proline, HArg represents homoarginine, PYA represents 4-pentynoic acid, 3,3-DPA represents 3,3-diphenylalanine, Cba represents β-cyclobutylalanine, 1Nal represents 1-naphthylalanine, hGlu represents homoglutamic acid, Thi represents thienyl-alanine, 4ThiAz represents beta-(4-thiazolyl)-alanine, His1Me represents N1-methyl-L-histidine, His3Me represents N3-methyl-L-histidine, 3Thi represents, Palmitoyl-Glu-LysN₃|PYA represents: 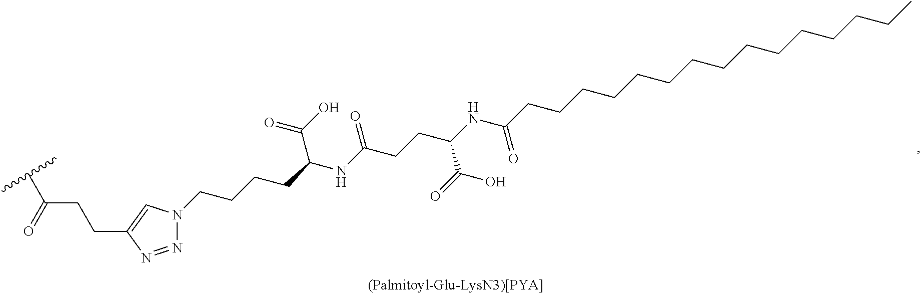 [SMILES: *C(=O)CCc1cn(CCCC[C@H](NC(=O)CC[C@H](NC(=O)CCCCCCCCCCCCCCC)C(=O)O)C(=O)O)nn1]
represents: 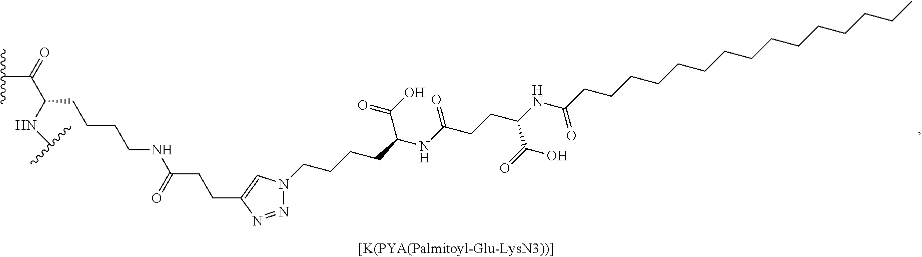 [SMILES: *N[C@@H](CCCCNC(=O)CCc1cn(CCCC[C@H](NC(=O)CC[C@H](NC(=O)CCCCCCCCCCCCCCC)C(=O)O)C(=O)O)nn1)C(*)=O]
Nle represents norleucine, MerPro represents 3-mercaptopropionic acid and Cysam represents cysteamine, or a pharmaceutically acceptable salt thereof.

16-17. (canceled)

18. The method of claim 14, wherein the EphA2 binding bicyclic peptide ligand comprises an amino acid sequence optionally comprising N-terminal modifications, wherein the amino acid sequence is selected from:
A-[HArg]-D-(SEQ ID NO: 24)
(herein referred to as BCY9594);
[B-Ala]-[Sar10]-A-[HArg]-D-(SEQ ID NO: 24)
(herein referred to as BCY6099);
[PYA]-A-[HArg]-D-(SEQ NO: 24)
(herein referred to as BCY11813);
Ac-A-[HArg]-D-(SEQ ID NO: 24)-[K(PYA)]
(herein referred to as BCY11814);
Ac-A-[HArg]-D-(SEQ ID NO: 24)-K
(herein referred to as BCY12734);
[NMeAla]-[HArg]-D-(SEQ ID NO: 24)
(herein referred to as BCY13121);
[Ac]-(SEQ ID NO: 24)-L[dH]G[dK]
(herein referred to as BCY13125);
[PYA]-[B-Ala]-[Sar10]-VGP-(SEQ ID NO: 25)
(herein referred to as BCY8941);
Ac-A-[HArg]-D-(SEQ ID NO: 26)
(herein referred to as BCY11815);
Ac-A-[HArg]-D-(SEQ ID NO: 27)
(herein referred to as BCY11816);
Ac-A-[HArg]-D-(SEQ ID NO: 28)
(herein referred to as BCY11817);
Ac-A-[HArg]-D-(SEQ ID NO: 29)
(herein referred to as BCY12735);
(Palmitoyl-Glu-LysN3)[PYA]A
[HArg]D-(SEQ ID NO: 29)
(hereinafter known as BCY14327);
Ac-A-[HArg]-D-(SEQ ID NO: 30)
(herein referred to as BCY12736);
Ac-A-[HArg]-D-(SEQ ID NO: 31)
(herein referred to as BCY12737);
A-[HArg]-D-(SEQ ID NO: 32)
(herein referred to as BCY12738);
A-[HArg]-E-(SEQ ID NO: 32)
(herein referred to as BCY12739);
A-[HArg]-D-(SEQ ID NO: 33)
(herein referred to as BCY12854);
A-[HArg]-D-(SEQ ID NO: 34)
(herein referred to as BCY12855);
A-[HArg]-D-(SEQ ID NO: 35)
(herein referred to as BCY12856);
A-[HArg]-D-(SEQ ID NO: 35)-[dA]
(herein referred to as BCY12857);
(SEQ ID NO: 35)-[dA]
(herein referred to as BCY12861);
[NMeAla]-[HArg]-D-(SEQ ID NO: 35)
(herein referred to as BCY13122);
[dA]-ED-(SEQ ID NO: 35)
(herein referred to as BCY13126);
[dA]-[dA]-D-(SEQ ID NO: 35)
(herein referred to as BCY13127);
AD-(SEQ ID NO: 35)
(herein referred to as BCY13128);
A-[HArg]-D-(SEQ ID NO: 36)
(herein referred to as BCY12858);
A-[HArg]-D-(SEQ ID NO: 37)
(herein referred to as BCY12859);
Ac-(SEQ ID NO: 37)-[dK]
(herein referred to as BCY13120);
A-[HArg]-D-(SEQ ID NO: 38)
(herein referred to as BCY12862);
A-[HArg]-D-(SEQ ID NO: 39)
(herein referred to as BCY12863);
[dA]-[HArg]-D-(SEQ ID NO: 39)-[dA]
(herein referred to as BCY12864);
(SEQ ID NO: 40)-[dA]
(herein referred to as BCY12865);
A-[HArg]-D-(SEQ ID NO: 41)
(herein referred to as BCY12866);
A-[HArg]-D-(SEQ ID NO: 42)
(herein referred to as BCY13116);
A-[HArg]-D-(SEQ ID NO: 43)
(herein referred to as BCY13117);
A-[HArg]-D-(SEQ ID NO: 44)
(herein referred to as BCY13118);
[dA]-[HArg]-D-(SEQ ID NO: 46)-[dA]
(herein referred to as BCY13123);
[d1Nal]-[HArg]-D-(SEQ ID NO: 46)-[dA]
(herein referred to as BCY13124);
A-[HArg]-D-(SEQ ID NO: 47)
(herein referred to as BCY13130);
A-[HArg]-D-(SEQ ID NO: 48)
(herein referred to as BCY13131);
A-[HArg]-D-(SEQ ID NO: 49)
(herein referred to as BCY13132);
A-[HArg]-D-(SEQ ID NO: 50)
(herein referred to as BCY13134);
A-[HArg]-D-(SEQ ID NO: 51)
(herein referred to as BCY13135);
(SEQ ID NO: 154)-[dK]
(herein referred to as BCY13129);
A[HArg]D-(SEQ ID NO: 155)
(herein referred to as BCY13133);
A[HArg]D-(SEQ ID NO: 156)
(herein referred to as BCY13917);
A[HArg]D-(SEQ ID NO: 157)
(herein referred to as BCY13918);
A[HArg]D-(SEQ ID NO: 158)
(herein referred to as BCY13919);
A[HArg]D-(SEQ ID NO: 159)
(herein referred to as BCY13920);
A[HArg]D-(SEQ ID NO: 160)
(herein referred to as BCY13922);
A[HArg]D-(SEQ ID NO: 161)
(herein referred to as BCY13923);
A[HArg]D-(SEQ ID NO: 162)
(herein referred to as BCY14047);
A[HArg]D-(SEQ ID NO: 163)
(herein referred to as BCY14048);
and
A[HArg]D-(SEQ ID NO: 164)
(herein referred to as BCY14313);
wherein PYA represents 4-pentynoic acid, B-Ala represents beta-alanine, Sar₁₀ represents 10 sarcosine units, HArg represents homoarginine, NMeAla represents N-methyl-alanine, 1Nal represents 1-naphthylalanine Palmitoyl-Glu-LysN₃[PYA] represents: 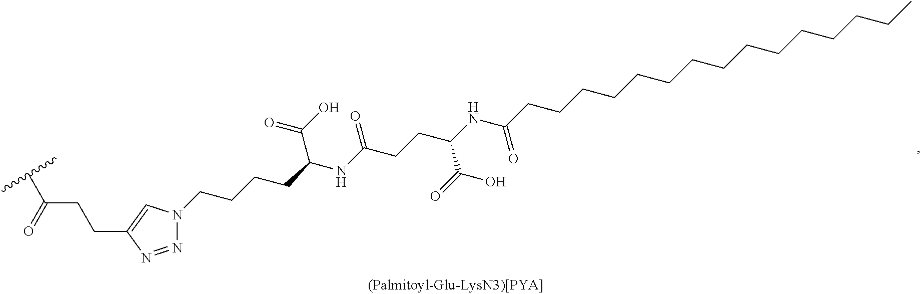 [SMILES: *C(=O)CCc1cn(CCCC[C@H](NC(=O)CC[C@H](NC(=O)CCCCCCCCCCCCCCC)C(=O)O)C(=O)O)nn1]
or a pharmaceutically acceptable salt thereof.

19-20. (canceled)

21. The method of claim 14, wherein the heterotandem bicyclic peptide complex is selected from those listed in Table C, such as BCY12491, BCY12730, BCY13048, BCY13050, BCY13053 and BCY13272, or a pharmaceutically acceptable salt thereof.

22. The method of claim 1, wherein the molecular scaffold is 1,1′,1″-(1,3,5-triazinane-1,3,5-triyl)triprop-2-en-1-one (TATA).

23. The method of claim 1, wherein the immuno-oncology agent is a checkpoint inhibitor.

24. The method of claim 1, wherein the immuno-oncology agent is an antagonist of CTLA-4, PD-1, PD-L1, PD-L2, LAG-3, TIM-3, Galectin 9, CEACAM-1, BTLA, CD69, Galectin-1, TIGIT, CD1113, GPR56, VISTA, 2B4, CD48, GARP, PD1H, LAIR1, TIM-1, or TIM-4.

25-45. (canceled)

46. The method of claim 1, wherein the cancer is a solid tumor.

47-65. (canceled)
